SEQUENCE LISTING (6 sequences)

SEQ ID NO: 1 - DNA, 20 bp, synthetic construct
  source 1..20: mol_type = other DNA
atggaacacagcgggaatca

SEQ ID NO: 2 - DNA, 21 bp, synthetic construct
  source 1..21: mol_type = other DNA
cagcaacagcagaaacatgga

SEQ ID NO: 3 - DNA, 15 bp, synthetic construct
  source 1..15: mol_type = other DNA
tgccctctgggaaag

SEQ ID NO: 4 - DNA, 20 bp, synthetic construct
  source 1..20: mol_type = other DNA
ccgcagtgggctaccataaa

SEQ ID NO: 5 - DNA, 20 bp, synthetic construct
  source 1..20: mol_type = other DNA
aatggcagggcaatgtagga

SEQ ID NO: 6 - DNA, 14 bp, synthetic construct
  source 1..14: mol_type = other DNA
tcccacgaggaagc

(19) United States
(12) Patent Application Publication
(10) Pub. No.: US 2025/0109442 A1
(43) Pub. Date: Apr. 3, 2025

(54) PROSTATE CANCER MARKERS AND USES THEREOF

(71) Applicants: Oslo Universitetssykehus HF, Oslo (NO); Universitetet i Oslo, Oslo (NO); Akershus Universitetssykehus HF, Lørenskog (NO)

(72) Inventors: Rolf I. Skotheim, Oslo (NO); Karol Axcrona, Bekkestua (NO); Ragnhild A. Lothe, Oslo (NO); Ulrika Axcrona, Bekkestua (NO); Jonas Meier Strømme, Oslo (NO); Bjarne Johannessen, Oslo (NO)

(21) Appl. No.: 18/728,416

(22) PCT Filed: Jan. 12, 2023
(86) PCT No.: PCT/IB2023/000012

§ 371 Date: Jul. 11, 2024

Related U.S. Application Data
(60) Provisional application No. 63/299,116, filed on Jan. 13, 2022.

(51) Int. Cl. C12Q 1/6886

(52) U.S. Cl. CPC C12Q 1/6886; C12Q 2600/106; C12Q 2600/112; C12Q 2600/118; C12Q 2600/158

(57) ABSTRACT

The present invention relates to compositions and methods for cancer diagnosis, research and therapy, including but not limited to, cancer markers. In particular, the present invention relates to markers for use in the diagnosis, prognosis, and treatment of prostate cancer.

### CROSS-REFERENCE TO RELATED APPLICATION

The present application claims priority to U.S. Provisional Patent Application No. 63/299,116, filed Jan. 13, 2022, which is hereby incorporated by reference in its entirety.

### FIELD OF THE INVENTION

The present invention relates to compositions and methods for cancer diagnosis, research and therapy, including but not limited to, cancer markers. In particular, the present invention relates to markers for use in the diagnosis, prognosis, and treatment of prostate cancer.

### BACKGROUND OF THE INVENTION

Prostate cancer is the commonest non-epithelial cancer in men in Norway, much of Western Europe and the USA. Approximately 9 million new cases are diagnosed worldwide annually and approximately 260,000 deaths occur due to prostate cancer, positioning it as the sixth leading cause of cancer-associated deaths. In men it represents the third most common cause of cancer-associated fatality after lung and colorectal cancer. Incidence in men under the age of 50 is rare and often associated strongly with family history and genetic predisposition. The incidence rises significantly with age and ethnicity has also been reported to be a significant risk factor in cohort studies. The five-year survival rate for localized disease is 100% however upon progression the survival rate drops to <50%.

Owing to the high-incidence of prostate cancer together with the prolonged time to progression (5-10 years) occurring in a minority of detected cases (˜20%), there is a pressing need for improved early detection and more robust risk stratification at the time of diagnosis. This is important to ensure that precious clinical resources are targeted as effectively as possible on those that will benefit most from primary treatment (surgery or radiotherapy) and may also benefit from the most intensive post-treatment follow-up and where necessary additional treatment upon recurrence (e.g., anti-androgens, androgen synthesis inhibitors, chemotherapy, beamline radiotherapy).

The optimal test for prostate cancer represents a panel of biomarkers with considerable specificity for prostate cancer combined with biomarkers predictive of metastatic progression and perhaps shared across cancer types due to conservation of biological process. A number of prostate-specific biomarkers have been reported. The earliest to be adopted clinically was prostate-specific antigen (PSA) which is now a mainstay as a blood test however because this marker is also expressed basally by non-cancerous prostate tissue its effective implementation requires the establishment of baseline levels for individuals, repeat testing and careful attention to decision thresholds (Green et al, J Urol. 2013 January; 189 (1 Suppl): S2-S11). Nonetheless, it is now often the gatekeeper test to needle biopsy and pathology grading (Gleason score) and staging of the disease. Subsequently other prostate cancer-specific biomarkers have been proposed to augment PSA testing and often are positioned to enhance the probability of identifying cancer on repeat biopsy. The markers that have advanced furthest in this setting are detected using PCR-based assays and are PCA3 (Gittelman et al, J Urol. 2013 July; 190 (1): 64-9) and the TMPRSS2-ERG gene fusion (Yao et al, Tumour Biol. 2014 March; 35 (3): 2157-66). The former is now clinically approved in the USA. By contrast, no progress has so far been made in adopting biomarkers for upfront prognostication into the clinical routine. This is despite the fact that in the absence of such markers there is a significant probability of expensive overtreatment of the disease in some settings due to its high incidence.

Additional markers for providing prostate cancer prognoses are needed.

### SUMMARY OF THE INVENTION

The present invention relates to compositions and methods for cancer diagnosis, research and therapy, including but not limited to, cancer markers. In particular, the present invention relates to markers for use in the diagnosis, prognosis, and treatment of prostate cancer.

Most patients with prostate cancer have multiple malignant tumour foci upon diagnosis. These foci typically show mutually exclusive somatic mutations demonstrated in a recent paper from our group (Løvf et al., Eur. Urol. 2019). We have recently shown that the same lesions have as different RNA expression patterns as if they derive from different patients (Strømme et al., Cancer Gene Therapy 2022). Through optimized biobanking protocol, long-term clinical follow-up data, transcriptome-wide RNA-seq data generation and an innovative data analysis strategy, we have identified 16 genes which expression levels are inter-focal heterogeneity-agnostic and informative on the patient's prognosis. This is demonstrated by the following results: The genes are (1) stably expressed within each patient (similar values between different malignant tumour foci of the same patient), (2) differentially expressed among cancer patients, and (3) have expression levels which are associated with time to biochemical relapse after prostatectomy.

Accordingly, in some embodiments, the present invention provides methods for providing a prognosis for a subject with prostate cancer, or selecting a subject with prostate cancer for treatment with a particular therapy, comprising: detecting the level of expression of one or more genes selected from the group consisting of ACOT1, ARHGEF35, C17orf97, CAPN9, CCDC163P, DDX58, DDX60, ERAP2, IFIH1, KLF13, MICA, NOMO3, PAM, THNSL2, TMC4, and USP18 in a sample from the subject; and comparing the level of expression of the one or more genes to a corresponding reference level of expression of the one or more genes, wherein an altered level of expression of the one or more genes relative to the reference level provides an indication selected from the group consisting of an indication of prostate cancer recurrence, an indication of survival of the subject, and an indication that the subject is a candidate for treatment with a particular therapy. In some preferred embodiments, an altered level of the genes in the sample as compared to the reference level is indicative of a decreased time to biochemical recurrence of prostate cancer in the subject.

In some preferred embodiments, the present invention provides methods for treating prostate cancer, comprising: detecting the level of expression of one or more genes selected from the group consisting of ACOT1, ARHGEF35, C17orf97, CAPN9, CCDC163P, DDX58, DDX60, ERAP2, IFIH1, KLF13, MICA, NOMO3, PAM, THNSL2, TMC4, and USP18 in a sample from the subject; and administering a prostate cancer treatment to a subject with an altered level of expression of the one or more genes.

In some preferred embodiments, the present invention provides methods for assaying gene expression in a sample from a subject diagnosed with prostate cancer, comprising: detecting the level of expression of two or more genes selected from the group consisting of ACOT1, ARHGEF35, C17orf97, CAPN9, CCDC163P, DDX58, DDX60, ERAP2, IFIH1, KLF13, MICA, NOMO3, PAM, THNSL2, TMC4, and USP18 in the sample.

In some preferred embodiments, the present invention provides methods for stratifying a subject with prostate cancer comprising: detecting the level of expression of one or more genes selected from the group consisting of ACOT1, ARHGEF35, C17orf97, CAPN9, CCDC163P, DDX58, DDX60, ERAP2, IFIH1, KLF13, MICA, NOMO3, PAM, THNSL2, TMC4, and USP18 in the sample; and assigning a risk of prostate cancer recurrence to the subject based upon detection of an altered level of expression of the one or more genes. In some preferred embodiments, the risk of prostate cancer recurrence is a higher risk of recurrence as compared to a subject not exhibiting an altered level of the one or more genes.

In some preferred embodiments of the foregoing methods, the altered level of expression is an increased or decreased level of expression. In some preferred embodiments, the altered level of expression is an increased level of expression. In some preferred embodiments, the altered level of expression is a decreased level of expression. In some preferred embodiments, the one or more genes is two or more. In some preferred embodiments, the one or more genes is 5 or more. In some preferred embodiments, the one or more genes is all of the genes. In some preferred embodiments, the sample is selected from the group consisting of prostate tissue, bone marrow, blood, serum, plasma, urine, prostatic fluid and semen. In some preferred embodiments, the prostate tissue is prostate cancer biopsy tissue. In some preferred embodiments, the sample comprises a prostate cancer cell.

In some preferred embodiments of the foregoing methods, the subject has undergone surgery and/or radiotherapy.

In some preferred embodiments of the foregoing methods, the detecting comprises the use of one or more nucleic acid reagents selected from the group consisting of a nucleic acid primers and nucleic acid probes and one or more antibodies. In some preferred embodiments, the primers, probes, and/or antibodies comprise a detectable label.

In some preferred embodiments of the foregoing methods, the subject has an already known high or intermediate risk of biochemical recurrence of cancer.

In some preferred embodiments of the foregoing methods, the methods further comprise stratifying the patients with an altered level of expression of one or more of the genes into an additional risk group.

In some preferred embodiments of the foregoing methods, the patient has previously undergone a radical prostatectomy.

In some preferred embodiments of the foregoing methods, the methods further comprise administering adjuvant treatment to the subject having an altered level expression of one or more of the genes. In some preferred embodiments, the adjuvant treatment is selected from the group consisting of chemotherapy and androgen deprivation therapy and combinations thereof. In some preferred embodiments, the androgen deprivation therapy comprises administration of a luteinizing hormone-releasing hormone (LHRH) or gonadotropin-releasing hormone (GnRH) agonist or antagonist. In some preferred embodiments, the luteinizing hormone-releasing hormone (LHRH) or gonadotropin-releasing hormone (GnRH) agonist or antagonist is selected from the group consisting of Leuprolide, Goserelin, Triptorelin, Histrelin, and Degarelix. In some preferred embodiments, the androgen deprivation therapy comprises administration of an antiandrogen. In some preferred embodiments, the antiandrogen is selected from the group consisting of Bicalutamide, Nilutamide, and Flutamide.

In some preferred embodiments, the present invention provides for use of a reagent that specifically detects an altered level of expression of one or more genes selected from the group consisting of ACOT1, ARHGEF35, C17orf97, CAPN9, CCDC163P, DDX58, DDX60, ERAP2, IFIH1, KLF13, MICA, NOMO3, PAM, THNSL2, TMC4, and USP18 in a sample from a subject in the determination of the likelihood of survival of the subject or determining that the subject is a candidate for treatment with a particular therapy.

In some preferred embodiments, the present invention provides a kit for detecting altered levels of genes expression in a sample from a subject, comprising: reagents that specifically detect two or more genes selected from the group consisting of ACOT1, ARHGEF35, C17orf97, CAPN9, CCDC163P, DDX58, DDX60, ERAP2, IFIH1, KLF13, MICA, NOMO3, PAM, THNSL2, TMC4, and USP18. In some preferred embodiments, the reagents are selected from the group consisting of nucleic acid primers and nucleic acid probes.

Additional embodiments are described herein.

### DESCRIPTION OF THE FIGURES

FIG. 1A-F: Principal component analysis shows heterogenous gene expression levels between different clinicopathological categories and between different prostate tissue samples from the same patients. As input for the analysis were the expression values of the 500 most varying genes and lncRNAs. In the upper row, samples are colored by a) tissue type (malignant vs. benign), b) ISUP grade group, and c) tissue proximity to extraprostatic extension. In the lower row, samples from one patient per plot are highlighted (patient 3 in d, patient 5 in e and patient 17 in f) and different red shapes represent samples from different malignant foci. The black square in e) represents a technical replicate of one of the samples from the focus represented with red squares. EPE: Extraprostatic extension; ISUP: International Society of Urological Pathology; GG: grade group.

FIG. 2: Expression of selected fusion genes with relevance to prostate cancer. Focus 1, 2 and 3 are denoted by yellow, orange, and red, respectively. Dark squares indicate expression, light squares indicate lack of expression.

FIG. 3: Distribution of expression status for 2115 mutations in 64 malignant prostate cancer samples. Each tick on the x-axis represents a sample. The top plot displays the relative frequency of the expression status for all mutations in a sample, while the bottom plot displays the total number of mutations. The center heatmap annotates which focus a sample is taken from (top row) and whether the TMPRSS2-ERG fusion gene is detected in the sample (bottom row).

FIG. 4A-B: Inter-patient and intra-patient heterogeneity scores. a) Heterogeneity quadrants derived from coefficients of variation of TPM gene expression values. b) Scatterplot of genes, with intraclass correlation coefficient on the x-axis, colored by ICC category. P-values from univariate Cox regression (−log₁₀) of BCR-free survival are on the y-axis, and the dashed line represents the significance threshold of p<0.05. Triangles represent genes with lower than average interpatient heterogeneity and higher than average intrapatient heterogeneity (Q4 in a) that have good or excellent ICC scores and adequate expression levels.

FIG. 5A-C: Genes differentially expressed in EPE vs. non-EPE malignant tissues from the same prostates. a) Volcano plot showing results from differential gene expression analysis of EPE and non-EPE malignant samples. b) Principal component analysis plot based on 60 significantly differentially expressed genes in EPE vs. non-EPE samples. c) The five most significantly up- and downregulated gene sets in EPE compared to non-EPE samples, ranked by normalized enrichment scores from the Hallmarks and Biological Processes gene set collections from MSigDB.

FIG. 6A-D: Genes differentially expressed in ETS positive vs. negative malignant tissues from the same prostates. a) Volcano plot showing results from DGEA of ETS positive and ETS negative malignant samples. b) Principal component analysis plot of 265 significantly differentially expressed genes in ETS positive (green dots) vs. ETS negative (white dots) samples. c) The five most significantly positively enriched gene sets in ETS positive samples compared to ETS negative samples in the Biological Processes and Hallmarks gene set collections. d) Overview of malignant tissue samples included in the EPE and ETS analyses, colored by phenotype.

FIG. 7: Ranked analysis of the 16 genes which had stable expression within a patient's prostate, varying expression between different patients, and univariately associated with BCR. Included expression data were from 249 patients with prostate cancer of Gleason grade 4+3 or higher from the cohort of The Cancer Genome Atlas. Each of the 16 genes were scored as contributing to good or bad prognosis in each of the 249 patients. The genes were ranked by how well they contributed to the correct prognosis. The analyses started by including the first-ranked gene, and stepwise including more genes. The Kaplan-Meier plots are examples of including 1, 4, 5, 6, 7, 10, 13, 15, and 16 genes, where the thresholds between two groups are at 0, 3, 4, 4, 5, 8, 10, 11, and 11 genes. The scatter plot shows the −log₁₀(p-values) as an increasing number of genes are included.

FIG. 8: Hazard ration by cox regression with stepwise addition of multiple genes from the 16 genes which had stable expression within a patient's prostate, varying expression between different patients, and univariately associated with BCR. Included expression data were from 249 patients with prostate cancer of Gleason grade 4+3 or higher from the cohort of The Cancer Genome Atlas. Each of the 16 genes were scored as contributing to good or bad prognosis in each of the 249 patients. The genes were ranked by how well they contributed to the correct prognosis. The analyses started by including the first-ranked gene, and stepwise including more genes. The Kaplan-Meier plots are examples of including 1, 4, 5, 6, 7, 10, 13, 15, and 16 genes, where the thresholds between two groups are at 0, 3, 4, 4, 5, 8, 10, 11, and 11 genes. The scatter plot shows the −Log₁₀(p-values) as an increasing number of genes are included.

FIG. 9: Kaplan-Meier plot of high vs. low risk prostate cancer as defined by a model produced by Ridge penalized regression. The expression level from all 16 genes from each of the 249 prostate cancer patients with Gleason 4+3 or higher in The Cancer Genome Atlas were used as input. An important parameter of constructing the model is lambda. The selection of the best lambda is optimized by cross validation, which is dependent on a random seed number. Thus, to find a robust lambda, the analysis was run 100 times with a different seed number each time. The median lambda from these 100 analyses was used further. A threshold for separating the samples into high or low-risk groups was set by the survminer tool. The Kaplan-Meier plot shows the fraction of patients without biochemical relapse (y-axis) according to time (days) after prostatectomy (x-axis).

FIG. 10: Amplification plot of ABL1 from a serial dilution of Universal Human Reference RNA. A threshold at the y-axis is given when the samples are in their maximum exponential phase. For each sample, the cycle number (x-axis) at which their amplification curve crosses the threshold is denoted the cycle threshold (Ct) value. This value is used as input in the further data processing. In the example amplification plot of samples used to produce the standard curve for ABL1, samples with input template ranging from 1 to 100 ng cDNA are included (Screen shot from the TaqMan software).

FIG. 11A-B: Distribution plots of significance values for association with BCR at a range of different thresholds. Standardized log-rank statistic (−Log₁₀ of p-value) are on the y-axis and expression values of the gene of interest on the x-axis. A. The gene ACOT1 is significant across a wide range of thresholds. B. The gene C17orf97 is significant at a more narrow range of thresholds.

FIG. 12A-B: Kaplan-Meier plots with all 247 patients. A. The 209 patients with low expression of ACOT1 were associated with significantly shorter time to BCR than the 38 samples with high expression (p=0.0066). B. The 35 patients with low expression of C17orf97 were associated with significantly shorter time to BCR than the 212 patients with high expression (p=0.00011).

FIG. 13A-B: Kaplan-Meier plots with 233 patients with high quality cDNA input. A. The 196 patients with low expression of ACOT1 were associated with significantly shorter time to BCR than the 37 patients with high expression (p=0.0027). B. The 84 patients with high expression of KLF13 were associated with significantly shorter time to BCR than the 149 samples with low expression (p=0.0029).

FIG. 14: Hazard ratios and confidence intervals for each of the 16 genes. Genes are sorted from highest to lowest hazard ratios. Parentheses represent the categorized expression level associated with the hazard ratios.

FIG. 15A-D: Patients scored by their number of genes with favourable expression, among 16 genes. For each of 233 patients, the count of favourable genes was made. A. Distribution of the number of patients (y-axis) with each of the numbers of favourable genes (x-axis). B-D. Kaplan-Meier plots patients with low risk of BCR scored when at least 8, 10, or 12 genes had favourable expression. The hazard ratios at these thresholds were 4.3, 3.2, and 4.3, respectively, and all had statistically significant different association with BCR than their respective high risk groups (p<0.0001).

FIG. 16A-D: Patients scored by their number of genes with favourable expression, among 10 genes. For each of 233 patients, the count of favourable genes was made. A. Distribution of the number of patients (y-axis) with each of the numbers of favourable genes (x-axis). B-D. Kaplan-Meier plots patients with low risk of BCR scored when at least 5, 6, or 8 genes had favourable expression. The hazard ratios at these thresholds were 4.2, 2.8, and 3.4, respectively, and all had statistically significant different association with BCR than their respective high risk groups (p<0.0001).

FIG. 17A-B: Association between gene expression and biochemical relapse in patients with intermediate and high-risk cancers (Gleason grade group of at least 3). A. The 131 patients with low expression of THNSL2 were associated with significantly shorter time to BCR than the 16 patients with high expression (p=0.01). B. The 21 patients with low expression of C17orf97 were associated with significantly shorter time to BCR than the 126 patients with high expression (p<0.0001).

FIG. 18: A-I: Principal components analysis indicating clustering of multiple samples per patient. Input data was expression values from 12 genes per malignant prostate tissue sample, for each of 412 malignant tissue samples. A-C. Individual plot for each of the three example patients described in the chapter below (the young [A], the miracle [B], and the unlucky [C]). A-I. The 9 patients, where the samples from the same patient are coloured in red. Different shapes indicate that the samples derive from different malignant foci.

### DEFINITIONS

To facilitate an understanding of the present invention, a number of terms and phrases are defined below:

As used herein, the terms “detect”, “detecting” or “detection” may describe either the general act of discovering or discerning or the specific observation of a detectably labeled composition.

As used herein, the term “subject” refers to any organisms that are screened using the diagnostic methods described herein. Such organisms preferably include, but are not limited to, mammals (e.g., murines, simians, equines, bovines, porcines, canines, felines, and the like), and most preferably includes humans.

The term “diagnosed,” as used herein, refers to the recognition of a disease by its signs and symptoms, or genetic analysis, pathological analysis, histological analysis, and the like.

A “subject suspected of having cancer” encompasses an individual who has received an initial diagnosis (e.g., a CT scan showing a mass or increased PSA level) but for whom the stage of cancer or gene expression levels indicative of cancer prognosis is not known. The term further includes people who once had cancer (e.g., an individual in remission). In some embodiments, “subjects” are control subjects that are suspected of having cancer or diagnosed with cancer.

As used herein, the term “characterizing cancer in a subject” refers to the identification of one or more properties of a cancer sample in a subject, including but not limited to, the presence of benign, pre-cancerous or cancerous tissue, the stage of the cancer, and the subject's prognosis.

Cancers may be characterized by the level of expression of genes described herein in cancer cells.

As used herein, the term “characterizing a prostate sample in a subject” refers to the identification of one or more properties of a prostate tissue sample (e.g., including but not limited to, the presence of cancerous tissue, the level of gene expression of genes described herein, the presence of pre-cancerous tissue that is likely to become cancerous, and the presence of cancerous tissue that is likely to metastasize, the presence of cancerous tissue that is likely to recur, or the likelihood of prostate cancer-specific death).

As used herein, the term “stage of cancer” refers to a qualitative or quantitative assessment of the level of advancement of a cancer. Criteria used to determine the stage of a cancer include, but are not limited to, the size of the tumor and the extent of metastases (e.g., localized or distant).

As used herein, the term “purified” or “to purify” refers to the removal of components (e.g., contaminants) from a sample.

As used herein, the term “sample” is used in its broadest sense. In one sense, it is meant to include a specimen or culture obtained from any source, as well as biological and environmental samples. Biological samples may be obtained from animals (including humans) and encompass fluids, solids, and tissues. Biological samples include blood products, such as plasma, serum and the like. Such examples are not however to be construed as limiting the sample types applicable to the present invention.

### DETAILED DESCRIPTION OF THE INVENTION

The present invention relates to compositions and methods for cancer diagnosis, research and therapy, including but not limited to, cancer markers. In particular, the present invention relates to markers for use in the diagnosis, prognosis, and treatment of prostate cancer.

Prostate cancer is a high-incidence male cancer with a significant age association and progression in a subset of diagnosed cases. In the absence of effective prognostication there are significant social and economic costs associated with overtreatment and unnecessary treatment. Furthermore, certain patients with the highest risk cancers would benefit from even more radical treatment. Improved upfront risk stratification would transform healthcare delivery and alleviate stresses on families, patients and medical practitioners.

Accordingly, in some embodiments, provided herein is a method for providing a prognosis for a subject with prostate cancer, or selecting a subject with prostate cancer for treatment with a particular therapy, comprising: (a) detecting the level of expression of one or more (e.g., 1, 2, 3, 4, 5, 6, 7, 8, 9, 10, 11, 12, 13, 14, 15, or all) genes selected from ACOT1, ARHGEF35, C17orf97, CAPN9, CCDC163P, DDX58, DDX60, ERAP2, IFIH1, KLF13, MICA, NOMO3, PAM, THNSL2, TMC4, or USP18 in a sample from said subject; and (b) comparing the level of expressing of the one or more genes to a reference level of expression of the genes, wherein an altered (e.g., increased or decreased) level of expression of the genes relative to the reference level provides an indication of disease recurrence, indication of survival of the subject, or an indication that the subject is a candidate for treatment with a particular therapy. For example, in some embodiments, an altered level of the genes is indicative of a decreased time to biochemical recurrence of prostate cancer in said subject.

The present invention is not limited to particular combinations of the listed markers. In some embodiments, the markers are two of the listed markers (e.g., ACOT1 and ARHGEF35; ACOT1 and C17orf97; ACOT1 and CAPN9; ACOT1 and CCDC163P; ACOT1 and DDX58; ACOT1 and DDX60; ACOT1 and ERAP2; ACOT1 and IFIH1; ACOT1 and KLF13; ACOT1 and MICA; ACOT1 and NOMO3; ACOT1 and PAM; ACOT1 and THNSL2; ACOT1 and TMC4; ACOT1 and USP18; ARHGEF35 and C17orf97; ARHGEF35 and CAPN9; ARHGEF35 and CCDC163P; ARHGEF35 and DDX58; ARHGEF35 and DDX60; ARHGEF35 and ERAP2; ARHGEF35 and IFIH1; ARHGEF35 and KLF13; ARHGEF35 and MICA; ARHGEF35 and NOMO3; PAM; ARHGEF35 and THNSL2 ARHGEF35 and TMC4; ARHGEF35 and USP18; C17orf97 and CAPN9; C17orf97 and CCDC163P C17orf97 and DDX58; C17orf97 and DDX60 C17orf97 and ERAP2; C17orf97 and IFIH1; C17orf97 and KLF13; C17orf97 and MICA C17orf97 and NOMO3; C17orf97 and PAM; C17orf97 and THNSL2; C17orf97 and TMC4; C17orf97 and USP18; CAPN9, CCDC163P, DDX58, DDX60, ERAP2, IFIH1, KLF13, MICA, NOMO3, PAM, THNSL2, TMC4, or USP18; CAPN9 and CCDC163P; CAPN9 and DDX58; CAPN9 and DDX60; CAPN9 and ERAP2; CAPN9 and IFIH1; CAPN9 and KLF13; CAPN9 and MICA; CAPN9 and NOMO3; CAPN9 and PAM; CAPN9 and THNSL2; CAPN9 and TMC4; CAPN9 and USP18; CCDC163P and DDX58; CCDC163P and DDX60; CCDC163P and ERAP2; CCDC163P and IFIH1; CCDC163P and KLF13; CCDC163P and MICA; CCDC163P and NOMO3; CCDC163P and PAM; CCDC163P and THNSL2; CCDC163P and TMC4; CCDC163P and USP18; DDX58 and DDX60; DDX58 and ERAP2; DDX58 and IFIH1; DDX58 and KLF13; DDX58 and MICA; DDX58 and NOMO3 DDX58 and PAM; DDX58 and THNSL2 DDX58 and TMC4; DDX58 and USP18; DDX60 and ERAP2; DDX60 and IFIH1; DDX60 and KLF13; DDX60 and MICA; DDX60 and NOMO3; DDX60 and PAM; DDX60 and THNSL2; DDX60 and TMC4; DDX60 and or USP18; ERAP2 and IFIH1; ERAP2 and KLF13; ERAP2 and MICA; ERPA2 and NOMO3; ERPA2 and PAM; ERAP2 and THNSL2; ERAP2 and TMC4; ERAP2 and USP18; IFIH1 and KLF13; IFIH1 and MICA; IFIH1 and NOMO3; IFIH1 and PAM; IFIH1 and THNSL2; IFIH1 and TMC4; IFIH1 and USP18; KLF13 and MICA; KLF13 and NOMO3; KLF13 and PAM; KLF13 and THNSL2; KLF13 and TMC4; KLF13 and USP18; MICA and NOMO3; MICA and PAM; MICA and THNSL2; MICA and TMC4; MICA and USP18; NOMO3 and PAM; NOMO3 and THNSL2; NOMO3 and TMC4; NOMO3 and USP18; PAM and THNSL2; PAM and TMC4; PAM and USP18; THNSL2 and TMC4; THNSL2 and USP18; or THNSL2 and TMC4).

In some embodiments, the markers are three or more of the markers (e.g., ACOT1, ARHGEF35, and C17orf97; ACOT1, ARHGEF35, and CAPN9; ACOT1, ARHGEF35, and CCDC163P; ACOT1, ARHGEF35, and DDX58; ACOT1, ARHGEF35, and DDX60; ACOT1, ARHGEF35, and ERAP2; ACOT1, ARHGEF35, and IFIH1; ACOT1, ARHGEF35, and KLF13; ACOT1, ARHGEF35, and MICA; ACOT1, ARHGEF35, and NOMO3; ACOT1, ARHGEF35, and PAM; ACOT1, ARHGEF35, and THNSL2; ACOT1, ARHGEF35, and TMC4; ACOT1, ARHGEF35, and USP18; ARHGEF35, C17orf97, and CAPN9; ARHGEF35, C17orf97, and CCDC163P; ARHGEF35, C17orf97, and DDX58; ARHGEF35, C17orf97, and DDX60; ARHGEF35, C17orf97, and ERAP2; ARHGEF35, C17orf97, and IFIH1; ARHGEF35, C17orf97, and KLF13; ARHGEF35, C17orf97, and MICA; ARHGEF35, C17orf97, and NOMO3; ARHGEF35, C17orf97, and PAM; ARHGEF35, C17orf97, and THNSL2; ARHGEF35, C17orf97, and TMC4; ARHGEF35, C17orf97, and USP18; C17orf97, CAPN9, and ACOT1; C17orf97, CAPN9, and ARHGEF35; C17orf97, CAPN9, and CCDC163P; C17orf97, CAPN9, and DDX58; C17orf97, CAPN9, and DDX60; C17orf97, CAPN9, and ERAP2; C17orf97, CAPN9, and IFIH1; C17orf97, CAPN9, and KLF13; C17orf97, CAPN9, and MICA; C17orf97, CAPN9, and NOMO3; C17orf97, CAPN9, and PAM; C17orf97, CAPN9, and THNSL2; C17orf97, CAPN9, and TMC4; C17orf97, CAPN9, and USP18; CAPN9, CCDC163P, and ACOT1; ARHGEF35; CAPN9, CCDC163P, and C17orf97; CAPN9, CCDC163P, and DDX58; CAPN9, CCDC163P, and DDX60 CAPN9, CCDC163P, and ERAP2; CAPN9, CCDC163P, and IFIH1; CAPN9, CCDC163P, and KLF13; CAPN9, CCDC163P, and MICA; CAPN9, CCDC163P, and NOMO3; CAPN9, CCDC163P, and PAM; CAPN9, CCDC163P, and THNSL2; CAPN9, CCDC163P, and TMC4; CAPN9, CCDC163P, and USP18; CCDC163P, DDX58, and ACOT1; CCDC163P, DDX58, and ARHGEF35; CCDC163P, DDX58, and C17orf97; CCDC163P, DDX58, and CAPN9; CCDC163P, DDX58, and DDX60; CCDC163P, DDX58, and ERAP2; CCDC163P, DDX58, and IFIH1; CCDC163P, DDX58, and KLF13; CCDC163P, DDX58, and MICA; CCDC163P, DDX58, and NOMO3; CCDC163P, DDX58, and PAM; T CCDC163P, DDX58, and HNSL2; CCDC163P, DDX58, and TMC4; CCDC163P, DDX58, and USP18; DDX58, DDX60, and ACOT1; ARHGEF35; DDX58, DDX60, and C17orf97; DDX58, DDX60, and CAPN9; DDX58, DDX60, and CCDC163P; DDX58, DDX60, and ERAP2; DDX58, DDX60, and IFIH1; DDX58, DDX60, and KLF13; DDX58, DDX60, and MICA; DDX58, DDX60, and NOMO3; DDX58, DDX60, and PAM; DDX58, DDX60, and THNSL2; DDX58, DDX60, and TMC4; DDX58, DDX60, and USP18; DDX60, ERAP2, and ACOT1; DDX60, ERAP2, and ARHGEF35; DDX60, ERAP2, and C17orf97; DDX60, ERAP2, and CAPN9; DDX60, ERAP2, and CCDC163P; DDX60, ERAP2, and DDX58; DDX60, ERAP2, and IFIH1; DDX60, ERAP2, and KLF13; DDX60, ERAP2, and MICA; DDX60, ERAP2, and NOMO3; DDX60, ERAP2, and PAM; DDX60, ERAP2, and THNSL2; DDX60, ERAP2, and TMC4; DDX60, ERAP2, and USP18; ERAP2, IFIH1, and ACOT1; ERAP2, IFIH1, and ARHGEF35; ERAP2, IFIH1, and C17orf97; ERAP2, IFIH1, and CAPN9; ERAP2, IFIH1, and CCDC163P; ERAP2, IFIH1, and DDX58; ERAP2, IFIH1, and DDX60; ERAP2, IFIH1, and KLF13; ERAP2, IFIH1, and MICA; ERAP2, IFIH1, and NOMO3; ERAP2, IFIH1, and PAM; ERAP2, IFIH1, and, THNSL2; ERAP2, IFIH1, and TMC4; ERAP2, IFIH1, and USP18; IFIH1, KLF13, and ACOT1; IFIH1, KLF13, and ARHGEF35; IFIH1, KLF13, and C17orf97; IFIH1, KLF13, and CAPN9; IFIH1, KLF13, and CCDC163P; IFIH1, KLF13, and DDX58; IFIH1, KLF13, and DDX60; IFIH1, KLF13, and ERAP2; IFIH1, KLF13, and MICA; IFIH1, KLF13, and NOMO3; IFIH1, KLF13, and PAM; IFIH1, KLF13, and THNSL2; IFIH1, KLF13, and TMC4; IFIH1, KLF13, and USP18; KLF13, MICA, and ACOT1; KLF13, MICA, and ARHGEF35; KLF13, MICA, and C17orf97; KLF13, MICA, and, CAPN9; KLF13, MICA, and CCDC163P; KLF13, MICA, and DDX58; KLF13, MICA, and DDX60; KLF13, MICA, and ERAP2; KLF13, MICA, and IFIH1; KLF13, MICA, and NOMO3; KLF13, MICA, and PAM; KLF13, MICA, and THNSL2; KLF13, MICA, and TMC4; KLF13, MICA, and USP18; MICA, NOMO3, and ACOT1; MICA, NOMO3, and ARHGEF35; MICA, NOMO3, and C17orf97; MICA, NOMO3, and CAPN9; MICA, NOMO3, and CCDC163P; MICA, NOMO3, and DDX58; MICA, NOMO3, and DDX60; MICA, NOMO3, and ERAP2; MICA, NOMO3, and IFIH1; MICA, NOMO3, and KLF13; MICA, NOMO3, and PAM; MICA, NOMO3, and THNSL2; MICA, NOMO3, and TMC4; MICA, NOMO3, and USP18; NOMO3, PAM and ACOT1; NOMO3, PAM and ARHGEF35; NOMO3, PAM and C17orf97; NOMO3, PAM and CAPN9; NOMO3, PAM and CCDC163P; NOMO3, PAM and DDX58; NOMO3, PAM and DDX60; NOMO3, PAM and ERAP2; NOMO3, PAM and IFIH1; NOMO3, PAM and KLF13; NOMO3, PAM and MICA; NOMO3, PAM and THNSL2; NOMO3, PAM and TMC4; NOMO3, PAM and USP18; PAM, THNSL2, and ACOT1; PAM, THNSL2, and ARHGEF35; PAM, THNSL2, and C17orf97; PAM, THNSL2, and CAPN9; PAM, THNSL2, and CCDC163P; PAM, THNSL2, and DDX58; PAM, THNSL2, and DDX60; PAM, THNSL2, and ERAP2; PAM, THNSL2, and IFIH1; PAM, THNSL2, and KLF13; PAM, THNSL2, and MICA; PAM, THNSL2, and NOMO3, PAM, THNSL2, and TMC4; PAM, THNSL2, and USP18; THNSL2, TMC4, and ACOT1; THNSL2, TMC4, and ARHGEF35; THNSL2, TMC4, and C17orf97; THNSL2, TMC4, and CAPN9; THNSL2, TMC4, and CCDC163P; THNSL2, TMC4, and DDX58; THNSL2, TMC4, and DDX60; THNSL2, TMC4, and ERAP2; THNSL2, TMC4, and IFIH1; THNSL2, TMC4, and KLF13; THNSL2, TMC4, and MICA; THNSL2, TMC4, and NOMO3; THNSL2, TMC4, and PAM; THNSL2, TMC4, and USP18; TMC4, USP18, and ACOT1; TMC4, USP18, and ARHGEF35; TMC4, USP18, and C17orf97; TMC4, USP18, and CAPN9; TMC4, USP18, and CCDC163P; TMC4, USP18, and DDX58; TMC4, USP18, and DDX60; TMC4, USP18, and ERAP2; TMC4, USP18, and IFIH1; TMC4, USP18, and KLF13; TMC4, USP18, and MICA; TMC4, USP18, and NOMO3; TMC4, USP18, and PAM).

In some embodiments, the markers are four or more of the markers (e.g., ACOT1, ARHGEF35, C17orf97, and CAPN9; ACOT1, ARHGEF35, C17orf97, and CCDC163P; ACOT1, ARHGEF35, C17orf97, and DDX58; ACOT1, ARHGEF35, C17orf97, and DDX60 ACOT1, ARHGEF35, C17orf97, and ERAP2; ACOT1, ARHGEF35, C17orf97, and IFIH1; ACOT1, ARHGEF35, C17orf97, and KLF13; ACOT1, ARHGEF35, C17orf97, and MICA; ACOT1, ARHGEF35, C17orf97, and NOMO3; ACOT1, ARHGEF35, C17orf97, and PAM; ACOT1, ARHGEF35, C17orf97, and THNSL2; ACOT1, ARHGEF35, C17orf97, and TMC4; ACOT1, ARHGEF35, C17orf97, and USP18; ARHGEF35, C17orf97, CAPN9, and ACOT1; ARHGEF35, C17orf97, CAPN9, and CCDC163P; ARHGEF35, C17orf97, CAPN9, and DDX58; ARHGEF35, C17orf97, CAPN9, and DDX60; ARHGEF35, C17orf97, CAPN9, and ERAP2; ARHGEF35, C17orf97, CAPN9, and IFIH1; ARHGEF35, C17orf97, CAPN9, and KLF13; ARHGEF35, C17orf97, CAPN9, and MICA; ARHGEF35, C17orf97, CAPN9, and NOMO3; ARHGEF35, C17orf97, CAPN9, and PAM; ARHGEF35, C17orf97, CAPN9, and THNSL2; ARHGEF35, C17orf97, CAPN9, and TMC4; ARHGEF35, C17orf97, CAPN9, and USP18; C17orf97, CAPN9, CCDC163P, and ACOT1; C17orf97, CAPN9, CCDC163P, and ARHGEF35; C17orf97, CAPN9, CCDC163P, and DDX58; C17orf97, CAPN9, CCDC163P, and DDX60; C17orf97, CAPN9, CCDC163P, and ERAP2; C17orf97, CAPN9, CCDC163P, and IFIH1; C17orf97, CAPN9, CCDC163P, and KLF13; C17orf97, CAPN9, CCDC163P, and MICA; C17orf97, CAPN9, CCDC163P, and NOMO3; C17orf97, CAPN9, CCDC163P, and PAM; C17orf97, CAPN9, CCDC163P, and THNSL2; C17orf97, CAPN9, CCDC163P, and TMC4; C17orf97, CAPN9, CCDC163P, and USP18; CAPN9, CCDC163P, DDX58 and ACOT1; CAPN9, CCDC163P, DDX58 and ARHGEF35; CAPN9, CCDC163P, DDX58 and C17orf97; CAPN9, CCDC163P, DDX58 and DDX60; CAPN9, CCDC163P, DDX58 and ERAP2; CAPN9, CCDC163P, DDX58 and IFIH1; CAPN9, CCDC163P, DDX58 and KLF13; CAPN9, CCDC163P, DDX58 and MICA; CAPN9, CCDC163P, DDX58 and NOMO3; CAPN9, CCDC163P, DDX58 and PAM; CAPN9, CCDC163P, DDX58 and THNSL2; CAPN9, CCDC163P, DDX58 and TMC4; CAPN9, CCDC163P, DDX58 and USP18; CCDC163P, DDX58, DDX60, and ACOT1; CCDC163P, DDX58, DDX60, and ARHGEF35; CCDC163P, DDX58, DDX60, and C17orf97; CCDC163P, DDX58, DDX60, and CAPN9; CCDC163P, DDX58, DDX60, and ERAP2; CCDC163P, DDX58, DDX60, and IFIH1; CCDC163P, DDX58, DDX60, and KLF13; CCDC163P, DDX58, DDX60, and MICA; CCDC163P, DDX58, DDX60, and NOMO3; CCDC163P, DDX58, DDX60, and PAM; CCDC163P, DDX58, DDX60, and THNSL2; CCDC163P, DDX58, DDX60, and TMC4; CCDC163P, DDX58, DDX60, and USP18; DDX58, DDX60, ERAP2, and ACOT1; DDX58, DDX60, ERAP2, and ARHGEF35; DDX58, DDX60, ERAP2, and C17orf97; DDX58, DDX60, ERAP2, and CAPN9; DDX58, DDX60, ERAP2, and CCDC163P; DDX58, DDX60, ERAP2, and IFIH1; DDX58, DDX60, ERAP2, and KLF13; DDX58, DDX60, ERAP2, and MICA; DDX58, DDX60, ERAP2, and NOMO3; DDX58, DDX60, ERAP2, and PAM; DDX58, DDX60, ERAP2, and THNSL2; DDX58, DDX60, ERAP2, and TMC4; DDX58, DDX60, ERAP2, and USP18; DDX60, ERAP2, IFIH1, and ACOT1; DDX60, ERAP2, IFIH1, and ARHGEF35; DDX60, ERAP2, IFIH1, and C17orf97; DDX60, ERAP2, IFIH1, and CAPN9; DDX60, ERAP2, IFIH1, and CCDC163P; DDX60, ERAP2, IFIH1, and DDX58, DDX60, ERAP2, IFIH1, and KLF13; DDX60, ERAP2, IFIH1, and MICA; DDX60, ERAP2, IFIH1, and NOMO3; DDX60, ERAP2, IFIH1, and PAM; DDX60, ERAP2, IFIH1, and THNSL2; DDX60, ERAP2, IFIH1, and TMC4; DDX60, ERAP2, IFIH1, and USP18; ERAP2, IFIH1, KLF13, and ACOT1; ERAP2, IFIH1, KLF13, and ARHGEF35; ERAP2, IFIH1, KLF13, and C17orf97; ERAP2, IFIH1, KLF13, and CAPN9; ERAP2, IFIH1, KLF13, and CCDC163P; ERAP2, IFIH1, KLF13, and DDX58 ERAP2, IFIH1, KLF13, and DDX60; ERAP2, IFIH1, KLF13, and MICA; ERAP2, IFIH1, KLF13, and NOMO3; ERAP2, IFIH1, KLF13, and PAM; ERAP2, IFIH1, KLF13, and THNSL2; ERAP2, IFIH1, KLF13, and TMC4; ERAP2, IFIH1, KLF13, and USP18; IFIH1, KLF13, MICA, and ACOT1; IFIH1, KLF13, MICA, and ARHGEF35; IFIH1, KLF13, MICA, and C17orf97; IFIH1, KLF13, MICA, and CAPN9; IFIH1, KLF13, MICA, and CCDC163P; IFIH1, KLF13, MICA, and DDX58; IFIH1, KLF13, MICA, and DDX60 IFIH1, KLF13, MICA, and ERAP2; IFIH1, KLF13, MICA, and NOMO3; IFIH1, KLF13, MICA, and PAM, IFIH1, KLF13, MICA, and THNSL2; IFIH1, KLF13, MICA, and TMC4; IFIH1, KLF13, MICA, and USP18; KLF13, MICA, NOMO3, and ACOT1; KLF13, MICA, NOMO3, and ARHGEF35; KLF13, MICA, NOMO3, and C17orf97; KLF13, MICA, NOMO3, and CAPN9; KLF13, MICA, NOMO3, and CCDC163P; KLF13, MICA, NOMO3, and DDX58; KLF13, MICA, NOMO3, and DDX60; KLF13, MICA, NOMO3, and ERAP2; KLF13, MICA, NOMO3, and IFIH1; KLF13, MICA, NOMO3, and PAM; KLF13, MICA, NOMO3, and THNSL2; KLF13, MICA, NOMO3, and TMC4; KLF13, MICA, NOMO3, and USP18; MICA, NOMO3, PAM, and ACOT1; MICA, NOMO3, PAM, and ARHGEF35; MICA, NOMO3, PAM, and C17orf97; MICA, NOMO3, PAM, and CAPN9; MICA, NOMO3, PAM, and CCDC163P; MICA, NOMO3, PAM, and DDX58; MICA, NOMO3, PAM, and DDX60; MICA, NOMO3, PAM, and ERAP2; MICA, NOMO3, PAM, and IFIH1; MICA, NOMO3, PAM, and KLF13; MICA, NOMO3, PAM, and THNSL2; MICA, NOMO3, PAM, and TMC4; MICA, NOMO3, PAM, and USP18; NOMO3, PAM, THNSL2, and ACOT1; NOMO3, PAM, THNSL2, and ARHGEF35; NOMO3, PAM, THNSL2, and C17orf97; NOMO3, PAM, THNSL2, and CAPN9; NOMO3, PAM, THNSL2, and CCDC163P; NOMO3, PAM, THNSL2, and DDX58; NOMO3, PAM, THNSL2, and DDX60; NOMO3, PAM, THNSL2, and ERAP2; NOMO3, PAM, THNSL2, and IFIH1; NOMO3, PAM, THNSL2, and KLF13; NOMO3, PAM, THNSL2, and MICA; NOMO3, PAM, THNSL2, and TMC4; NOMO3, PAM, THNSL2, and USP18; PAM, THNSL2, TMC4, and ACOT1; PAM, THNSL2, TMC4, and ARHGEF35; PAM, THNSL2, TMC4, and C17orf97; PAM, THNSL2, TMC4, and CAPN9; PAM, THNSL2, TMC4, and CCDC163P; PAM, THNSL2, TMC4, and DDX58; PAM, THNSL2, TMC4, and DDX60; PAM, THNSL2, TMC4, and ERAP2; PAM, THNSL2, TMC4, and IFIH1; PAM, THNSL2, TMC4, and KLF13; PAM, THNSL2, TMC4, and MICA PAM, THNSL2, TMC4, and NOMO3; PAM, THNSL2, TMC4, and USP18; THNSL2, TMC4, USP18, and ARHGEF35; THNSL2, TMC4, USP18, and C17orf97; THNSL2, TMC4, USP18, and CAPN9; THNSL2, TMC4, USP18, and CCDC163P; THNSL2, TMC4, USP18, and DDX58; THNSL2, TMC4, USP18, and DDX60; THNSL2, TMC4, USP18, and ERAP2; THNSL2, TMC4, USP18, and IFIH1; THNSL2, TMC4, USP18, and KLF13; THNSL2, TMC4, USP18, and MICA; THNSL2, TMC4, USP18, and NOMO3; THNSL2, TMC4, USP18, and PAM; THNSL2, TMC4, USP18, and ACOT1).

In some embodiments, the markers are five or more of the markers (e.g., ACOT1, ARHGEF35, C17orf97, CAPN9, and CCDC163P; ACOT1, ARHGEF35, C17orf97, CAPN9, and DDX58; ACOT1, ARHGEF35, C17orf97, CAPN9, and DDX60; ACOT1, ARHGEF35, C17orf97, CAPN9, and ERAP2; ACOT1, ARHGEF35, C17orf97, CAPN9, and IFIH1; ACOT1, ARHGEF35, C17orf97, CAPN9, and KLF13; ACOT1, ARHGEF35, C17orf97, CAPN9, and, MICA; ACOT1, ARHGEF35, C17orf97, CAPN9, and NOMO3; ACOT1, ARHGEF35, C17orf97, CAPN9, and PAM; ACOT1, ARHGEF35, C17orf97, CAPN9, and THNSL2; ACOT1, ARHGEF35, C17orf97, CAPN9, and TMC4; ACOT1, ARHGEF35, C17orf97, CAPN9, and USP18; ARHGEF35, C17orf97, CAPN9, CCDC163P, and ACOT1; ARHGEF35, C17orf97, CAPN9, CCDC163P, and DDX58; ARHGEF35, C17orf97, CAPN9, CCDC163P, and DDX60; ARHGEF35, C17orf97, CAPN9, CCDC163P, and ERAP2; ARHGEF35, C17orf97, CAPN9, CCDC163P, and IFIH1; ARHGEF35, C17orf97, CAPN9, CCDC163P, and KLF13; ARHGEF35, C17orf97, CAPN9, CCDC163P, and MICA; ARHGEF35, C17orf97, CAPN9, CCDC163P, and NOMO3; ARHGEF35, C17orf97, CAPN9, CCDC163P, and PAM; ARHGEF35, C17orf97, CAPN9, CCDC163P, and THNSL2; ARHGEF35, C17orf97, CAPN9, CCDC163P, and TMC4; ARHGEF35, C17orf97, CAPN9, CCDC163P, and USP18; C17orf97, CAPN9, CCDC163P, DDX58 and ACOT1; C17orf97, CAPN9, CCDC163P, DDX58 and ARHGEF35; C17orf97, CAPN9, CCDC163P, DDX58 and DDX60; C17orf97, CAPN9, CCDC163P, DDX58 and ERAP2; C17orf97, CAPN9, CCDC163P, DDX58 and IFIH1; C17orf97, CAPN9, CCDC163P, DDX58 and KLF13; C17orf97, CAPN9, CCDC163P, DDX58 and MICA; C17orf97, CAPN9, CCDC163P, DDX58 and NOMO3; C17orf97, CAPN9, CCDC163P, DDX58 and PAM; C17orf97, CAPN9, CCDC163P, DDX58 and THNSL2; C17orf97, CAPN9, CCDC163P, DDX58 and TMC4; C17orf97, CAPN9, CCDC163P, DDX58 and USP18; CAPN9, CCDC163P, DDX58, DDX60, and ACOT1; CAPN9, CCDC163P, DDX58, DDX60, and ARHGEF35; CAPN9, CCDC163P, DDX58, DDX60, and C17orf97; CAPN9, CCDC163P, DDX58, DDX60, and ERAP2; CAPN9, CCDC163P, DDX58, DDX60, and IFIH1; CAPN9, CCDC163P, DDX58, DDX60, and KLF13; CAPN9, CCDC163P, DDX58, DDX60, and MICA; CAPN9, CCDC163P, DDX58, DDX60, and NOMO3; CAPN9, CCDC163P, DDX58, DDX60, and PAM; CAPN9, CCDC163P, DDX58, DDX60, and THNSL2; CAPN9, CCDC163P, DDX58, DDX60, and TMC4; CAPN9, CCDC163P, DDX58, DDX60, and USP18; CCDC163P, DDX58, DDX60, ERAP2, and ACOT1; CCDC163P, DDX58, DDX60, ERAP2, and ARHGEF35; CCDC163P, DDX58, DDX60, ERAP2, and C17orf97; CCDC163P, DDX58, DDX60, ERAP2, and CAPN9; CCDC163P, DDX58, DDX60, ERAP2, and IFIH1; CCDC163P, DDX58, DDX60, ERAP2, and KLF13; CCDC163P, DDX58, DDX60, ERAP2, and MICA; CCDC163P, DDX58, DDX60, ERAP2, and NOMO3; CCDC163P, DDX58, DDX60, ERAP2, and PAM; CCDC163P, DDX58, DDX60, ERAP2, and THNSL2; CCDC163P, DDX58, DDX60, ERAP2, and TMC4; CCDC163P, DDX58, DDX60, ERAP2, and USP18; DDX58, DDX60, ERAP2, IFIH1, and ACOT1; DDX58, DDX60, ERAP2, IFIH1, and ARHGEF35; DDX58, DDX60, ERAP2, IFIH1, and C17orf97; DDX58, DDX60, ERAP2, IFIH1, and CAPN9; DDX58, DDX60, ERAP2, IFIH1, and CCDC163P; DDX58, DDX60, ERAP2, IFIH1, and KLF13; DDX58, DDX60, ERAP2, IFIH1, and MICA; DDX58, DDX60, ERAP2, IFIH1, and NOMO3; DDX58, DDX60, ERAP2, IFIH1, and PAM; DDX58, DDX60, ERAP2, IFIH1, and THNSL2; DDX58, DDX60, ERAP2, IFIH1, and TMC4; DDX58, DDX60, ERAP2, IFIH1, and USP18; DDX60, ERAP2, IFIH1, KLF13, and ACOT1; DDX60, ERAP2, IFIH1, KLF13, and ARHGEF35; DDX60, ERAP2, IFIH1, KLF13, and C17orf97; DDX60, ERAP2, IFIH1, KLF13, and CAPN9; DDX60, ERAP2, IFIH1, KLF13, and CCDC163P; DDX60, ERAP2, IFIH1, KLF13, and DDX58; DDX60, ERAP2, IFIH1, KLF13, and MICA; DDX60, ERAP2, IFIH1, KLF13, and NOMO3; DDX60, ERAP2, IFIH1, KLF13, and PAM; DDX60, ERAP2, IFIH1, KLF13, and THNSL2; DDX60, ERAP2, IFIH1, KLF13, and TMC4; DDX60, ERAP2, IFIH1, KLF13, and USP18; ERAP2, IFIH1, KLF13, MICA, and ACOT1; ERAP2, IFIH1, KLF13, MICA, and ARHGEF35; ERAP2, IFIH1, KLF13, MICA, and C17orf97; ERAP2, IFIH1, KLF13, MICA, and CAPN9; ERAP2, IFIH1, KLF13, MICA, and CCDC163P; ERAP2, IFIH1, KLF13, MICA, and DDX58; ERAP2, IFIH1, KLF13, MICA, and DDX60; ERAP2, IFIH1, KLF13, MICA, and NOMO3; ERAP2, IFIH1, KLF13, MICA, and PAM; ERAP2, IFIH1, KLF13, MICA, and THNSL2; ERAP2, IFIH1, KLF13, MICA, and TMC4; ERAP2, IFIH1, KLF13, MICA, and USP18; IFIH1, KLF13, MICA, NOMO3, and ACOT1; IFIH1, KLF13, MICA, NOMO3, and ARHGEF35; IFIH1, KLF13, MICA, NOMO3, and C17orf97; IFIH1, KLF13, MICA, NOMO3, and CAPN9; IFIH1, KLF13, MICA, NOMO3, and CCDC163P; IFIH1, KLF13, MICA, NOMO3, and DDX58; IFIH1, KLF13, MICA, NOMO3, and DDX60; IFIH1, KLF13, MICA, NOMO3, and ERAP2; IFIH1, KLF13, MICA, NOMO3, and PAM; IFIH1, KLF13, MICA, NOMO3, and THNSL2; IFIH1, KLF13, MICA, NOMO3, and TMC4; IFIH1, KLF13, MICA, NOMO3, and USP18; KLF13, MICA, NOMO3, PAM, and ACOT1; KLF13, MICA, NOMO3, PAM, and ARHGEF35; KLF13, MICA, NOMO3, PAM, and C17orf97; KLF13, MICA, NOMO3, PAM, and CAPN9; KLF13, MICA, NOMO3, PAM, and CCDC163P; KLF13, MICA, NOMO3, PAM, and DDX58; KLF13, MICA, NOMO3, PAM, and DDX60; KLF13, MICA, NOMO3, PAM, and ERAP2 KLF13, MICA, NOMO3, PAM, and IFIH1; KLF13, MICA, NOMO3, PAM, and THNSL2; KLF13, MICA, NOMO3, PAM, and TMC4; KLF13, MICA, NOMO3, PAM, and USP18; MICA, NOMO3, PAM, THNSL2, and ACOT1; MICA, NOMO3, PAM, THNSL2, and ARHGEF35; MICA, NOMO3, PAM, THNSL2, and C17orf97; MICA, NOMO3, PAM, THNSL2, and CAPN9; MICA, NOMO3, PAM, THNSL2, and CCDC163P; MICA, NOMO3, PAM, THNSL2, and DDX58; MICA, NOMO3, PAM, THNSL2, and DDX60; MICA, NOMO3, PAM, THNSL2, and ERAP2; MICA, NOMO3, PAM, THNSL2, and IFIH1; MICA, NOMO3, PAM, THNSL2, and KLF13; MICA, NOMO3, PAM, THNSL2, and MICA; MICA, NOMO3, PAM, THNSL2, and NOMO3; MICA, NOMO3, PAM, THNSL2, and PAM; THNSL2, and TMC4; MICA, NOMO3, PAM, THNSL2, and USP18; MICA, NOMO3, PAM, THNSL2, and ACOT1; MICA, NOMO3, PAM, THNSL2, and ARHGEF35; MICA, NOMO3, PAM, THNSL2, and C17orf97; MICA, NOMO3, PAM, THNSL2, and CAPN9; MICA, NOMO3, PAM, THNSL2, and CCDC163P; MICA, NOMO3, PAM, THNSL2, and DDX58; MICA, NOMO3, PAM, THNSL2, and DDX60; MICA, NOMO3, PAM, THNSL2, and ERAP2 MICA, NOMO3, PAM, THNSL2, and IFIH1; MICA, NOMO3, PAM, THNSL2, and KLF13; MICA, NOMO3, PAM, THNSL2, and MICA; NOMO3, PAM, THNSL2, TMC4, and USP18).

In some embodiments, the markers are six or more of the markers (e.g., ACOT1, ARHGEF35, C17orf97, CAPN9, CCDC163P, and DDX58; ACOT1, ARHGEF35, C17orf97, CAPN9, CCDC163P, and DDX60; ACOT1, ARHGEF35, C17orf97, CAPN9, CCDC163P, and ERAP2; ACOT1, ARHGEF35, C17orf97, CAPN9, CCDC163P, and IFIH1; ACOT1, ARHGEF35, C17orf97, CAPN9, CCDC163P, and KLF13; ACOT1, ARHGEF35, C17orf97, CAPN9, CCDC163P, and MICA; ACOT1, ARHGEF35, C17orf97, CAPN9, CCDC163P, and NOMO3; ACOT1, ARHGEF35, C17orf97, CAPN9, CCDC163P, and PAM; ACOT1, ARHGEF35, C17orf97, CAPN9, CCDC163P, and THNSL2; ACOT1, ARHGEF35, C17orf97, CAPN9, CCDC163P, and TMC4; ACOT1, ARHGEF35, C17orf97, CAPN9, CCDC163P, and USP18; ARHGEF35, C17orf97, CAPN9, CCDC163P, DDX58, and ACOT1; ARHGEF35, C17orf97, CAPN9, CCDC163P, DDX58, and DDX60; ARHGEF35, C17orf97, CAPN9, CCDC163P, DDX58, and ERAP2; ARHGEF35, C17orf97, CAPN9, CCDC163P, DDX58, and IFIH1; ARHGEF35, C17orf97, CAPN9, CCDC163P, DDX58, and KLF13; ARHGEF35, C17orf97, CAPN9, CCDC163P, DDX58, and MICA; ARHGEF35, C17orf97, CAPN9, CCDC163P, DDX58, and NOMO3; ARHGEF35, C17orf97, CAPN9, CCDC163P, DDX58, and PAM; ARHGEF35, C17orf97, CAPN9, CCDC163P, DDX58, and THNSL2; ARHGEF35, C17orf97, CAPN9, CCDC163P, DDX58, and TMC4; ARHGEF35, C17orf97, CAPN9, CCDC163P, DDX58, and USP18; C17orf97, CAPN9, CCDC163P, DDX58, DDX60, and ACOT1; C17orf97, CAPN9, CCDC163P, DDX58, DDX60, and ARHGEF35; C17orf97, CAPN9, CCDC163P, DDX58, DDX60, and ERAP2; C17orf97, CAPN9, CCDC163P, DDX58, DDX60, and IFIH1; C17orf97, CAPN9, CCDC163P, DDX58, DDX60, and KLF13; C17orf97, CAPN9, CCDC163P, DDX58, DDX60, and MICA; C17orf97, CAPN9, CCDC163P, DDX58, DDX60, and NOMO3; C17orf97, CAPN9, CCDC163P, DDX58, DDX60, and PAM; C17orf97, CAPN9, CCDC163P, DDX58, DDX60, and THNSL2; C17orf97, CAPN9, CCDC163P, DDX58, DDX60, and TMC4; C17orf97, CAPN9, CCDC163P, DDX58, DDX60, and USP18; CAPN9, CCDC163P, DDX58, DDX60, ERAP2, and ACOT1; CAPN9, CCDC163P, DDX58, DDX60, ERAP2, and ARHGEF35; CAPN9, CCDC163P, DDX58, DDX60, ERAP2, and C17orf97; CAPN9, CCDC163P, DDX58, DDX60, ERAP2, and IFIH1; CAPN9, CCDC163P, DDX58, DDX60, ERAP2, and KLF13; CAPN9, CCDC163P, DDX58, DDX60, ERAP2, and MICA; CAPN9, CCDC163P, DDX58, DDX60, ERAP2, and NOMO3; CAPN9, CCDC163P, DDX58, DDX60, ERAP2, and PAM; CAPN9, CCDC163P, DDX58, DDX60, ERAP2, and THNSL2; CAPN9, CCDC163P, DDX58, DDX60, ERAP2, and TMC4; CAPN9, CCDC163P, DDX58, DDX60, ERAP2, and USP18; CCDC163P, DDX58, DDX60, ERAP2, IFIH1, and ACOT1; CCDC163P, DDX58, DDX60, ERAP2, IFIH1, and ARHGEF35; CCDC163P, DDX58, DDX60, ERAP2, IFIH1, and C17orf97; CCDC163P, DDX58, DDX60, ERAP2, IFIH1, and CAPN9; CCDC163P, DDX58, DDX60, ERAP2, IFIH1, and KLF13; CCDC163P, DDX58, DDX60, ERAP2, IFIH1, and MICA; CCDC163P, DDX58, DDX60, ERAP2, IFIH1, and NOMO3; CCDC163P, DDX58, DDX60, ERAP2, IFIH1, and PAM; CCDC163P, DDX58, DDX60, ERAP2, IFIH1, and THNSL2; CCDC163P, DDX58, DDX60, ERAP2, IFIH1, and TMC4; CCDC163P, DDX58, DDX60, ERAP2, IFIH1, and USP18; DDX58, DDX60, ERAP2, IFIH1, KLF13, and ACOT1; DDX58, DDX60, ERAP2, IFIH1, KLF13, and ARHGEF35; DDX58, DDX60, ERAP2, IFIH1, KLF13, and C17orf97; DDX58, DDX60, ERAP2, IFIH1, KLF13, and CAPN9; DDX58, DDX60, ERAP2, IFIH1, KLF13, and CCDC163P; DDX58, DDX60, ERAP2, IFIH1, KLF13, and MICA; DDX58, DDX60, ERAP2, IFIH1, KLF13, and NOMO3; DDX58, DDX60, ERAP2, IFIH1, KLF13, and PAM; DDX58, DDX60, ERAP2, IFIH1, KLF13, and THNSL2; DDX58, DDX60, ERAP2, IFIH1, KLF13, and TMC4; DDX58, DDX60, ERAP2, IFIH1, KLF13, and USP18; DDX60, ERAP2, IFIH1, KLF13, MICA, and ACOT1; DDX60, ERAP2, IFIH1, KLF13, MICA, and ARHGEF35; DDX60, ERAP2, IFIH1, KLF13, MICA, and C17orf97; DDX60, ERAP2, IFIH1, KLF13, MICA, and CAPN9; DDX60, ERAP2, IFIH1, KLF13, MICA, and CCDC163P; DDX60, ERAP2, IFIH1, KLF13, MICA, and DDX58; DDX60, ERAP2, IFIH1, KLF13, MICA, and NOMO3; DDX60, ERAP2, IFIH1, KLF13, MICA, and PAM; DDX60, ERAP2, IFIH1, KLF13, MICA, and THNSL2; DDX60, ERAP2, IFIH1, KLF13, MICA, and TMC4; DDX60, ERAP2, IFIH1, KLF13, MICA, and USP18; ERAP2, IFIH1, KLF13, MICA, NOMO3, and ACOT1; ERAP2, IFIH1, KLF13, MICA, NOMO3, and ARHGEF35; ERAP2, IFIH1, KLF13, MICA, NOMO3, and C17orf97; ERAP2, IFIH1, KLF13, MICA, NOMO3, and CAPN9; ERAP2, IFIH1, KLF13, MICA, NOMO3, and CCDC163P; ERAP2, IFIH1, KLF13, MICA, NOMO3, and DDX58; ERAP2, IFIH1, KLF13, MICA, NOMO3, and DDX60; ERAP2, IFIH1, KLF13, MICA, NOMO3, and PAM; ERAP2, IFIH1, KLF13, MICA, NOMO3, and THNSL2; ERAP2, IFIH1, KLF13, MICA, NOMO3, and TMC4; ERAP2, IFIH1, KLF13, MICA, NOMO3, and USP18; IFIH1, KLF13, MICA, NOMO3, PAM, and ACOT1; IFIH1, KLF13, MICA, NOMO3, PAM, and ARHGEF35; IFIH1, KLF13, MICA, NOMO3, PAM, and C17orf97; IFIH1, KLF13, MICA, NOMO3, PAM, and CAPN9; IFIH1, KLF13, MICA, NOMO3, PAM, and CCDC163P; IFIH1, KLF13, MICA, NOMO3, PAM, and DDX58, DDX60, ERAP2; IFIH1, KLF13, MICA, NOMO3, PAM, and THNSL2; IFIH1, KLF13, MICA, NOMO3, PAM, and TMC4; IFIH1, KLF13, MICA, NOMO3, PAM, and USP18; KLF13, MICA, NOMO3, PAM, THNSL2, and ACOT1; KLF13, MICA, NOMO3, PAM, THNSL2, and ARHGEF35; KLF13, MICA, NOMO3, PAM, THNSL2, and C17orf97; KLF13, MICA, NOMO3, PAM, THNSL2, and CAPN9; KLF13, MICA, NOMO3, PAM, THNSL2, and CCDC163P; KLF13, MICA, NOMO3, PAM, THNSL2, and DDX58; KLF13, MICA, NOMO3, PAM, THNSL2, and DDX60; KLF13, MICA, NOMO3, PAM, THNSL2, and ERAP2; KLF13, MICA, NOMO3, PAM, THNSL2, and IFIH1; KLF13, MICA, NOMO3, PAM, THNSL2, and TMC4; KLF13, MICA, NOMO3, PAM, THNSL2, and USP18; MICA, NOMO3, PAM, THNSL2, TMC4, and ACOT1; MICA, NOMO3, PAM, THNSL2, TMC4, and ARHGEF35; MICA, NOMO3, PAM, THNSL2, TMC4, and C17orf97; MICA, NOMO3, PAM, THNSL2, TMC4, and CAPN9; MICA, NOMO3, PAM, THNSL2, TMC4, and CCDC163P; MICA, NOMO3, PAM, THNSL2, TMC4, and DDX58; MICA, NOMO3, PAM, THNSL2, TMC4, and DDX60; MICA, NOMO3, PAM, THNSL2, TMC4, and ERAP2; MICA, NOMO3, PAM, THNSL2, TMC4, and IFIH1; MICA, NOMO3, PAM, THNSL2, TMC4, and KLF13; MICA, NOMO3, PAM, THNSL2, TMC4, and USP18).

In some embodiments, the markers are seven or more of the markers (e.g., ACOT1, ARHGEF35, C17orf97, CAPN9, CCDC163P, DDX58, and DDX60; ACOT1, ARHGEF35, C17orf97, CAPN9, CCDC163P, DDX58, and ERAP2; ACOT1, ARHGEF35, C17orf97, CAPN9, CCDC163P, DDX58, and IFIH1; ACOT1, ARHGEF35, C17orf97, CAPN9, CCDC163P, DDX58, and KLF13; ACOT1, ARHGEF35, C17orf97, CAPN9, CCDC163P, DDX58, and MICA; ACOT1, ARHGEF35, C17orf97, CAPN9, CCDC163P, DDX58, and NOMO3; ACOT1, ARHGEF35, C17orf97, CAPN9, CCDC163P, DDX58, and PAM; ACOT1, ARHGEF35, C17orf97, CAPN9, CCDC163P, DDX58, and THNSL2; ACOT1, ARHGEF35, C17orf97, CAPN9, CCDC163P, DDX58, and TMC4; ACOT1, ARHGEF35, C17orf97, CAPN9, CCDC163P, DDX58, and USP18; ARHGEF35, C17orf97, CAPN9, CCDC163P, DDX58, DDX60, and ACOT1; ARHGEF35, C17orf97, CAPN9, CCDC163P, DDX58, DDX60, and ERAP2; ARHGEF35, C17orf97, CAPN9, CCDC163P, DDX58, DDX60, and IFIH1; ARHGEF35, C17orf97, CAPN9, CCDC163P, DDX58, DDX60, and KLF13; ARHGEF35, C17orf97, CAPN9, CCDC163P, DDX58, DDX60, and MICA; ARHGEF35, C17orf97, CAPN9, CCDC163P, DDX58, DDX60, and NOMO3; ARHGEF35, C17orf97, CAPN9, CCDC163P, DDX58, DDX60, and PAM; ARHGEF35, C17orf97, CAPN9, CCDC163P, DDX58, DDX60, and THNSL2; ARHGEF35, C17orf97, CAPN9, CCDC163P, DDX58, DDX60, and TMC4; ARHGEF35, C17orf97, CAPN9, CCDC163P, DDX58, DDX60, and USP18; C17orf97, CAPN9, CCDC163P, DDX58, DDX60, ERAP2, and ACOT1; C17orf97, CAPN9, CCDC163P, DDX58, DDX60, ERAP2, and ARHGEF35; C17orf97, CAPN9, CCDC163P, DDX58, DDX60, ERAP2, and IFIH1; C17orf97, CAPN9, CCDC163P, DDX58, DDX60, ERAP2, and KLF13; C17orf97, CAPN9, CCDC163P, DDX58, DDX60, ERAP2, and MICA; C17orf97, CAPN9, CCDC163P, DDX58, DDX60, ERAP2, and NOMO3; C17orf97, CAPN9, CCDC163P, DDX58, DDX60, ERAP2, and PAM; C17orf97, CAPN9, CCDC163P, DDX58, DDX60, ERAP2, and THNSL2; C17orf97, CAPN9, CCDC163P, DDX58, DDX60, ERAP2, and TMC4; C17orf97, CAPN9, CCDC163P, DDX58, DDX60, ERAP2, and USP18; CAPN9, CCDC163P, DDX58, DDX60, ERAP2, IFIH1, and ACOT1; CAPN9, CCDC163P, DDX58, DDX60, ERAP2, IFIH1, and ARHGEF35; CAPN9, CCDC163P, DDX58, DDX60, ERAP2, IFIH1, and C17orf97; CAPN9, CCDC163P, DDX58, DDX60, ERAP2, IFIH1, and KLF13; CAPN9, CCDC163P, DDX58, DDX60, ERAP2, IFIH1, and MICA; CAPN9, CCDC163P, DDX58, DDX60, ERAP2, IFIH1, and NOMO3; CAPN9, CCDC163P, DDX58, DDX60, ERAP2, IFIH1, and PAM; CAPN9, CCDC163P, DDX58, DDX60, ERAP2, IFIH1, and THNSL2; CAPN9, CCDC163P, DDX58, DDX60, ERAP2, IFIH1, and TMC4; CAPN9, CCDC163P, DDX58, DDX60, ERAP2, IFIH1, and USP18; CCDC163P, DDX58, DDX60, ERAP2, IFIH1, KLF13, and ACOT1; CCDC163P, DDX58, DDX60, ERAP2, IFIH1, KLF13, and ARHGEF35; CCDC163P, DDX58, DDX60, ERAP2, IFIH1, KLF13, and C17orf97; CCDC163P, DDX58, DDX60, ERAP2, IFIH1, KLF13, and CAPN9; CCDC163P, DDX58, DDX60, ERAP2, IFIH1, KLF13, and MICA; CCDC163P, DDX58, DDX60, ERAP2, IFIH1, KLF13, and NOMO3; CCDC163P, DDX58, DDX60, ERAP2, IFIH1, KLF13, and PAM; CCDC163P, DDX58, DDX60, ERAP2, IFIH1, KLF13, and THNSL2; CCDC163P, DDX58, DDX60, ERAP2, IFIH1, KLF13, and TMC4; CCDC163P, DDX58, DDX60, ERAP2, IFIH1, KLF13, and USP18; DDX58, DDX60, ERAP2, IFIH1, KLF13, MICA, and ACOT1; DDX58, DDX60, ERAP2, IFIH1, KLF13, MICA, and ARHGEF35; DDX58, DDX60, ERAP2, IFIH1, KLF13, MICA, and C17orf97; DDX58, DDX60, ERAP2, IFIH1, KLF13, MICA, and CAPN9; DDX58, DDX60, ERAP2, IFIH1, KLF13, MICA, and CCDC163P; DDX58, DDX60, ERAP2, IFIH1, KLF13, MICA, and NOMO3; DDX58, DDX60, ERAP2, IFIH1, KLF13, MICA, and PAM; DDX58, DDX60, ERAP2, IFIH1, KLF13, MICA, and THNSL2; DDX58, DDX60, ERAP2, IFIH1, KLF13, MICA, and TMC4; DDX58, DDX60, ERAP2, IFIH1, KLF13, MICA, and USP18; DDX60, ERAP2, IFIH1, KLF13, MICA, NOMO3, and ACOT1; DDX60, ERAP2, IFIH1, KLF13, MICA, NOMO3, and ARHGEF35; DDX60, ERAP2, IFIH1, KLF13, MICA, NOMO3, and C17orf97; DDX60, ERAP2, IFIH1, KLF13, MICA, NOMO3, and CAPN9; DDX60, ERAP2, IFIH1, KLF13, MICA, NOMO3, and CCDC163P; DDX60, ERAP2, IFIH1, KLF13, MICA, NOMO3, and DDX58; DDX60, ERAP2, IFIH1, KLF13, MICA, NOMO3, and PAM; DDX60, ERAP2, IFIH1, KLF13, MICA, NOMO3, and THNSL2; DDX60, ERAP2, IFIH1, KLF13, MICA, NOMO3, and TMC4; DDX60, ERAP2, IFIH1, KLF13, MICA, NOMO3, and USP18; ERAP2, IFIH1, KLF13, MICA, NOMO3, PAM, and ACOT1; ERAP2, IFIH1, KLF13, MICA, NOMO3, PAM, and ARHGEF35; ERAP2, IFIH1, KLF13, MICA, NOMO3, PAM, and C17orf97; ERAP2, IFIH1, KLF13, MICA, NOMO3, PAM, and CAPN9; ERAP2, IFIH1, KLF13, MICA, NOMO3, PAM, and CCDC163P; ERAP2, IFIH1, KLF13, MICA, NOMO3, PAM, and DDX58; ERAP2, IFIH1, KLF13, MICA, NOMO3, PAM, and DDX60; ERAP2, IFIH1, KLF13, MICA, NOMO3, PAM, and THNSL2; ERAP2, IFIH1, KLF13, MICA, NOMO3, PAM, and TMC4; ERAP2, IFIH1, KLF13, MICA, NOMO3, PAM, and USP18; IFIH1, KLF13, MICA, NOMO3, PAM, THNSL2, and ACOT1; IFIH1, KLF13, MICA, NOMO3, PAM, THNSL2, and ARHGEF35; IFIH1, KLF13, MICA, NOMO3, PAM, THNSL2, and C17orf97; IFIH1, KLF13, MICA, NOMO3, PAM, THNSL2, and CAPN9; IFIH1, KLF13, MICA, NOMO3, PAM, THNSL2, and CCDC163P; IFIH1, KLF13, MICA, NOMO3, PAM, THNSL2, and DDX58; IFIH1, KLF13, MICA, NOMO3, PAM, THNSL2, and DDX60; IFIH1, KLF13, MICA, NOMO3, PAM, THNSL2, and ERAP2; IFIH1, KLF13, MICA, NOMO3, PAM, THNSL2, and TMC4; IFIH1, KLF13, MICA, NOMO3, PAM, THNSL2, and USP18; KLF13, MICA, NOMO3, PAM, THNSL2, TMC4, and ACOT1; KLF13, MICA, NOMO3, PAM, THNSL2, TMC4, and ARHGEF35; KLF13, MICA, NOMO3, PAM, THNSL2, TMC4, and C17orf97; KLF13, MICA, NOMO3, PAM, THNSL2, TMC4, and CAPN9; KLF13, MICA, NOMO3, PAM, THNSL2, TMC4, and CCDC163P; KLF13, MICA, NOMO3, PAM, THNSL2, TMC4, and DDX58; KLF13, MICA, NOMO3, PAM, THNSL2, TMC4, and DDX60; KLF13, MICA, NOMO3, PAM, THNSL2, TMC4, and ERAP2; KLF13, MICA, NOMO3, PAM, THNSL2, TMC4, and IFIH1; KLF13, MICA, NOMO3, PAM, THNSL2, TMC4, and USP18).

In some embodiments, the markers are eight or more of the markers (e.g., ACOT1, ARHGEF35, C17orf97, CAPN9, CCDC163P, DDX58, DDX60, and ERAP2; ACOT1, ARHGEF35, C17orf97, CAPN9, CCDC163P, DDX58, DDX60, and IFIH1; ACOT1, ARHGEF35, C17orf97, CAPN9, CCDC163P, DDX58, DDX60, and KLF13; ACOT1, ARHGEF35, C17orf97, CAPN9, CCDC163P, DDX58, DDX60, and MICA; ACOT1, ARHGEF35, C17orf97, CAPN9, CCDC163P, DDX58, DDX60, and NOMO3; ACOT1, ARHGEF35, C17orf97, CAPN9, CCDC163P, DDX58, DDX60, and PAM; ACOT1, ARHGEF35, C17orf97, CAPN9, CCDC163P, DDX58, DDX60, and THNSL2; ACOT1, ARHGEF35, C17orf97, CAPN9, CCDC163P, DDX58, DDX60, and TMC4; ACOT1, ARHGEF35, C17orf97, CAPN9, CCDC163P, DDX58, DDX60, and USP18; ARHGEF35, C17orf97, CAPN9, CCDC163P, DDX58, DDX60, ERAP2, and ACOT1; ARHGEF35, C17orf97, CAPN9, CCDC163P, DDX58, DDX60, ERAP2, and IFIH1; ARHGEF35, C17orf97, CAPN9, CCDC163P, DDX58, DDX60, ERAP2, and KLF13; ARHGEF35, C17orf97, CAPN9, CCDC163P, DDX58, DDX60, ERAP2, and MICA; ARHGEF35, C17orf97, CAPN9, CCDC163P, DDX58, DDX60, ERAP2, and NOMO3; ARHGEF35, C17orf97, CAPN9, CCDC163P, DDX58, DDX60, ERAP2, and PAM; ARHGEF35, C17orf97, CAPN9, CCDC163P, DDX58, DDX60, ERAP2, and THNSL2; ARHGEF35, C17orf97, CAPN9, CCDC163P, DDX58, DDX60, ERAP2, and TMC4; ARHGEF35, C17orf97, CAPN9, CCDC163P, DDX58, DDX60, ERAP2, and USP18; C17orf97, CAPN9, CCDC163P, DDX58, DDX60, ERAP2, IFIH1, and ACOT1; C17orf97, CAPN9, CCDC163P, DDX58, DDX60, ERAP2, IFIH1, and ARHGEF35; C17orf97, CAPN9, CCDC163P, DDX58, DDX60, ERAP2, IFIH1, and KLF13; C17orf97, CAPN9, CCDC163P, DDX58, DDX60, ERAP2, IFIH1, and MICA; C17orf97, CAPN9, CCDC163P, DDX58, DDX60, ERAP2, IFIH1, and NOMO3; C17orf97, CAPN9, CCDC163P, DDX58, DDX60, ERAP2, IFIH1, and PAM; C17orf97, CAPN9, CCDC163P, DDX58, DDX60, ERAP2, IFIH1, and THNSL2; C17orf97, CAPN9, CCDC163P, DDX58, DDX60, ERAP2, IFIH1, and TMC4; C17orf97, CAPN9, CCDC163P, DDX58, DDX60, ERAP2, IFIH1, and USP18; CAPN9, CCDC163P, DDX58, DDX60, ERAP2, IFIH1, KLF13, and ACOT1; CAPN9, CCDC163P, DDX58, DDX60, ERAP2, IFIH1, KLF13, and ARHGEF35; CAPN9, CCDC163P, DDX58, DDX60, ERAP2, IFIH1, KLF13, and C17orf97; CAPN9, CCDC163P, DDX58, DDX60, ERAP2, IFIH1, KLF13, and MICA; CAPN9, CCDC163P, DDX58, DDX60, ERAP2, IFIH1, KLF13, and NOMO3; CAPN9, CCDC163P, DDX58, DDX60, ERAP2, IFIH1, KLF13, and PAM; CAPN9, CCDC163P, DDX58, DDX60, ERAP2, IFIH1, KLF13, and THNSL2; CAPN9, CCDC163P, DDX58, DDX60, ERAP2, IFIH1, KLF13, and TMC4; CAPN9, CCDC163P, DDX58, DDX60, ERAP2, IFIH1, KLF13, and USP18; CCDC163P, DDX58, DDX60, ERAP2, IFIH1, KLF13, MICA, and ACOT1; CCDC163P, DDX58, DDX60, ERAP2, IFIH1, KLF13, MICA, and ARHGEF35; CCDC163P, DDX58, DDX60, ERAP2, IFIH1, KLF13, MICA, and C17orf97; CCDC163P, DDX58, DDX60, ERAP2, IFIH1, KLF13, MICA, and CAPN9; CCDC163P, DDX58, DDX60, ERAP2, IFIH1, KLF13, MICA, and NOMO3; CCDC163P, DDX58, DDX60, ERAP2, IFIH1, KLF13, MICA, and PAM; CCDC163P, DDX58, DDX60, ERAP2, IFIH1, KLF13, MICA, and THNSL2; CCDC163P, DDX58, DDX60, ERAP2, IFIH1, KLF13, MICA, and TMC4; CCDC163P, DDX58, DDX60, ERAP2, IFIH1, KLF13, MICA, and USP18; DDX58, DDX60, ERAP2, IFIH1, KLF13, MICA, NOMO3, and ACOT1; DDX58, DDX60, ERAP2, IFIH1, KLF13, MICA, NOMO3, and ARHGEF35; DDX58, DDX60, ERAP2, IFIH1, KLF13, MICA, NOMO3, and C17orf97; DDX58, DDX60, ERAP2, IFIH1, KLF13, MICA, NOMO3, and CAPN9; DDX58, DDX60, ERAP2, IFIH1, KLF13, MICA, NOMO3, and CCDC163P; DDX58, DDX60, ERAP2, IFIH1, KLF13, MICA, NOMO3, and PAM; DDX58, DDX60, ERAP2, IFIH1, KLF13, MICA, NOMO3, and THNSL2; DDX58, DDX60, ERAP2, IFIH1, KLF13, MICA, NOMO3, and TMC4; DDX58, DDX60, ERAP2, IFIH1, KLF13, MICA, NOMO3, and USP18; DDX60, ERAP2, IFIH1, KLF13, MICA, NOMO3, PAM, and ACOT1; DDX60, ERAP2, IFIH1, KLF13, MICA, NOMO3, PAM, and ARHGEF35; DDX60, ERAP2, IFIH1, KLF13, MICA, NOMO3, PAM, and C17orf97; DDX60, ERAP2, IFIH1, KLF13, MICA, NOMO3, PAM, and CAPN9; DDX60, ERAP2, IFIH1, KLF13, MICA, NOMO3, PAM, and CCDC163P; DDX60, ERAP2, IFIH1, KLF13, MICA, NOMO3, PAM, and DDX58; DDX60, ERAP2, IFIH1, KLF13, MICA, NOMO3, PAM, and THNSL2; DDX60, ERAP2, IFIH1, KLF13, MICA, NOMO3, PAM, and TMC4; DDX60, ERAP2, IFIH1, KLF13, MICA, NOMO3, PAM, and USP18; ERAP2, IFIH1, KLF13, MICA, NOMO3, PAM, THNSL2, and ACOT1; ERAP2, IFIH1, KLF13, MICA, NOMO3, PAM, THNSL2, and ARHGEF35; ERAP2, IFIH1, KLF13, MICA, NOMO3, PAM, THNSL2, and C17orf97; ERAP2, IFIH1, KLF13, MICA, NOMO3, PAM, THNSL2, and CAPN9; ERAP2, IFIH1, KLF13, MICA, NOMO3, PAM, THNSL2, and CCDC163P; ERAP2, IFIH1, KLF13, MICA, NOMO3, PAM, THNSL2, and DDX58; ERAP2, IFIH1, KLF13, MICA, NOMO3, PAM, THNSL2, and DDX60; ERAP2, IFIH1, KLF13, MICA, NOMO3, PAM, THNSL2, and TMC4; ERAP2, IFIH1, KLF13, MICA, NOMO3, PAM, THNSL2, and USP18; IFIH1, KLF13, MICA, NOMO3, PAM, THNSL2, TMC4, and ACOT1; IFIH1, KLF13, MICA, NOMO3, PAM, THNSL2, TMC4, and ARHGEF35; IFIH1, KLF13, MICA, NOMO3, PAM, THNSL2, TMC4, and C17orf97; IFIH1, KLF13, MICA, NOMO3, PAM, THNSL2, TMC4, and CAPN9; IFIH1, KLF13, MICA, NOMO3, PAM, THNSL2, TMC4, and CCDC163P; IFIH1, KLF13, MICA, NOMO3, PAM, THNSL2, TMC4, and, DDX58; IFIH1, KLF13, MICA, NOMO3, PAM, THNSL2, TMC4, and DDX60; IFIH1, KLF13, MICA, NOMO3, PAM, THNSL2, TMC4, and ERAP2; IFIH1, KLF13, MICA, NOMO3, PAM, THNSL2, TMC4, and USP18).

In some embodiments, the markers are nine or more of the markers (e.g., ACOT1, ARHGEF35, C17orf97, CAPN9, CCDC163P, DDX58, DDX60, ERAP2, and IFIH1; ACOT1, ARHGEF35, C17orf97, CAPN9, CCDC163P, DDX58, DDX60, ERAP2, and KLF13; ACOT1, ARHGEF35, C17orf97, CAPN9, CCDC163P, DDX58, DDX60, ERAP2, and MICA; ACOT1, ARHGEF35, C17orf97, CAPN9, CCDC163P, DDX58, DDX60, ERAP2, and NOMO3; ACOT1, ARHGEF35, C17orf97, CAPN9, CCDC163P, DDX58, DDX60, ERAP2, and PAM; ACOT1, ARHGEF35, C17orf97, CAPN9, CCDC163P, DDX58, DDX60, ERAP2, and THNSL2; ACOT1, ARHGEF35, C17orf97, CAPN9, CCDC163P, DDX58, DDX60, ERAP2, and TMC4; ACOT1, ARHGEF35, C17orf97, CAPN9, CCDC163P, DDX58, DDX60, ERAP2, and USP18; ARHGEF35, C17orf97, CAPN9, CCDC163P, DDX58, DDX60, ERAP2, IFIH1, and ACOT1; ARHGEF35, C17orf97, CAPN9, CCDC163P, DDX58, DDX60, ERAP2, IFIH1, and KLF13; ARHGEF35, C17orf97, CAPN9, CCDC163P, DDX58, DDX60, ERAP2, IFIH1, and MICA; ARHGEF35, C17orf97, CAPN9, CCDC163P, DDX58, DDX60, ERAP2, IFIH1, and NOMO3; ARHGEF35, C17orf97, CAPN9, CCDC163P, DDX58, DDX60, ERAP2, IFIH1, and PAM; ARHGEF35, C17orf97, CAPN9, CCDC163P, DDX58, DDX60, ERAP2, IFIH1, and THNSL2; ARHGEF35, C17orf97, CAPN9, CCDC163P, DDX58, DDX60, ERAP2, IFIH1, and TMC4; ARHGEF35, C17orf97, CAPN9, CCDC163P, DDX58, DDX60, ERAP2, IFIH1, and USP18; C17orf97, CAPN9, CCDC163P, DDX58, DDX60, ERAP2, IFIH1, KLF13, and ACOT1; C17orf97, CAPN9, CCDC163P, DDX58, DDX60, ERAP2, IFIH1, KLF13, and ARHGEF35; C17orf97, CAPN9, CCDC163P, DDX58, DDX60, ERAP2, IFIH1, KLF13, and MICA; C17orf97, CAPN9, CCDC163P, DDX58, DDX60, ERAP2, IFIH1, KLF13, and NOMO3; C17orf97, CAPN9, CCDC163P, DDX58, DDX60, ERAP2, IFIH1, KLF13, and PAM; C17orf97, CAPN9, CCDC163P, DDX58, DDX60, ERAP2, IFIH1, KLF13, and THNSL2; C17orf97, CAPN9, CCDC163P, DDX58, DDX60, ERAP2, IFIH1, KLF13, and TMC4; C17orf97, CAPN9, CCDC163P, DDX58, DDX60, ERAP2, IFIH1, KLF13, and USP18; CAPN9, CCDC163P, DDX58, DDX60, ERAP2, IFIH1, KLF13, MICA, and ACOT1; CAPN9, CCDC163P, DDX58, DDX60, ERAP2, IFIH1, KLF13, MICA, and ARHGEF35; CAPN9, CCDC163P, DDX58, DDX60, ERAP2, IFIH1, KLF13, MICA, and C17orf97; CAPN9, CCDC163P, DDX58, DDX60, ERAP2, IFIH1, KLF13, MICA, and NOMO3; CAPN9, CCDC163P, DDX58, DDX60, ERAP2, IFIH1, KLF13, MICA, and PAM; CAPN9, CCDC163P, DDX58, DDX60, ERAP2, IFIH1, KLF13, MICA, and THNSL2; CAPN9, CCDC163P, DDX58, DDX60, ERAP2, IFIH1, KLF13, MICA, and TMC4; CAPN9, CCDC163P, DDX58, DDX60, ERAP2, IFIH1, KLF13, MICA, and USP18; CCDC163P, DDX58, DDX60, ERAP2, IFIH1, KLF13, MICA, NOMO3, and ACOT1; CCDC163P, DDX58, DDX60, ERAP2, IFIH1, KLF13, MICA, NOMO3, and ARHGEF35; CCDC163P, DDX58, DDX60, ERAP2, IFIH1, KLF13, MICA, NOMO3, and C17orf97; CCDC163P, DDX58, DDX60, ERAP2, IFIH1, KLF13, MICA, NOMO3, and CAPN9; CCDC163P, DDX58, DDX60, ERAP2, IFIH1, KLF13, MICA, NOMO3, and PAM; CCDC163P, DDX58, DDX60, ERAP2, IFIH1, KLF13, MICA, NOMO3, and THNSL2; CCDC163P, DDX58, DDX60, ERAP2, IFIH1, KLF13, MICA, NOMO3, and TMC4; CCDC163P, DDX58, DDX60, ERAP2, IFIH1, KLF13, MICA, NOMO3, and USP18; DDX58, DDX60, ERAP2, IFIH1, KLF13, MICA, NOMO3, PAM, and ACOT1; DDX58, DDX60, ERAP2, IFIH1, KLF13, MICA, NOMO3, PAM, and ARHGEF35; DDX58, DDX60, ERAP2, IFIH1, KLF13, MICA, NOMO3, PAM, and C17orf97; DDX58, DDX60, ERAP2, IFIH1, KLF13, MICA, NOMO3, PAM, and CAPN9; DDX58, DDX60, ERAP2, IFIH1, KLF13, MICA, NOMO3, PAM, and CCDC163P; DDX58, DDX60, ERAP2, IFIH1, KLF13, MICA, NOMO3, PAM, and THNSL2; DDX58, DDX60, ERAP2, IFIH1, KLF13, MICA, NOMO3, PAM, and TMC4; DDX58, DDX60, ERAP2, IFIH1, KLF13, MICA, NOMO3, PAM, and USP18; DDX60, ERAP2, IFIH1, KLF13, MICA, NOMO3, PAM, THNSL2, and ACOT1; DDX60, ERAP2, IFIH1, KLF13, MICA, NOMO3, PAM, THNSL2, and ARHGEF35; DDX60, ERAP2, IFIH1, KLF13, MICA, NOMO3, PAM, THNSL2, and C17orf97; DDX60, ERAP2, IFIH1, KLF13, MICA, NOMO3, PAM, THNSL2, and CAPN9; DDX60, ERAP2, IFIH1, KLF13, MICA, NOMO3, PAM, THNSL2, and CCDC163P; DDX60, ERAP2, IFIH1, KLF13, MICA, NOMO3, PAM, THNSL2, and DDX58; DDX60, ERAP2, IFIH1, KLF13, MICA, NOMO3, PAM, THNSL2, and TMC4; DDX60, ERAP2, IFIH1, KLF13, MICA, NOMO3, PAM, THNSL2, and USP18; ERAP2, IFIH1, KLF13, MICA, NOMO3, PAM, THNSL2, TMC4, and ACOT1; ERAP2, IFIH1, KLF13, MICA, NOMO3, PAM, THNSL2, TMC4, and ARHGEF35; ERAP2, IFIH1, KLF13, MICA, NOMO3, PAM, THNSL2, TMC4, and C17orf97; ERAP2, IFIH1, KLF13, MICA, NOMO3, PAM, THNSL2, TMC4, and CAPN9; ERAP2, IFIH1, KLF13, MICA, NOMO3, PAM, THNSL2, TMC4, and CCDC163P; ERAP2, IFIH1, KLF13, MICA, NOMO3, PAM, THNSL2, TMC4, and DDX58; ERAP2, IFIH1, KLF13, MICA, NOMO3, PAM, THNSL2, TMC4, and DDX60; ERAP2, IFIH1, KLF13, MICA, NOMO3, PAM, THNSL2, TMC4, and USP18).

In some embodiments, the markers are ten or more of the markers (e.g., ACOT1, ARHGEF35, C17orf97, CAPN9, CCDC163P, DDX58, DDX60, ERAP2, IFIH1, and KLF13; ACOT1, ARHGEF35, C17orf97, CAPN9, CCDC163P, DDX58, DDX60, ERAP2, IFIH1, and MICA; ACOT1, ARHGEF35, C17orf97, CAPN9, CCDC163P, DDX58, DDX60, ERAP2, IFIH1, and NOMO3; ACOT1, ARHGEF35, C17orf97, CAPN9, CCDC163P, DDX58, DDX60, ERAP2, IFIH1, and PAM; ACOT1, ARHGEF35, C17orf97, CAPN9, CCDC163P, DDX58, DDX60, ERAP2, IFIH1, and THNSL2; ACOT1, ARHGEF35, C17orf97, CAPN9, CCDC163P, DDX58, DDX60, ERAP2, IFIH1, and TMC4; ACOT1, ARHGEF35, C17orf97, CAPN9, CCDC163P, DDX58, DDX60, ERAP2, IFIH1, and USP18; ARHGEF35, C17orf97, CAPN9, CCDC163P, DDX58, DDX60, ERAP2, IFIH1, KLF13, and ACOT1; ARHGEF35, C17orf97, CAPN9, CCDC163P, DDX58, DDX60, ERAP2, IFIH1, KLF13, and MICA; ARHGEF35, C17orf97, CAPN9, CCDC163P, DDX58, DDX60, ERAP2, IFIH1, KLF13, and NOMO3; ARHGEF35, C17orf97, CAPN9, CCDC163P, DDX58, DDX60, ERAP2, IFIH1, KLF13, and PAM; ARHGEF35, C17orf97, CAPN9, CCDC163P, DDX58, DDX60, ERAP2, IFIH1, KLF13, and THNSL2; ARHGEF35, C17orf97, CAPN9, CCDC163P, DDX58, DDX60, ERAP2, IFIH1, KLF13, and TMC4; ARHGEF35, C17orf97, CAPN9, CCDC163P, DDX58, DDX60, ERAP2, IFIH1, KLF13, and USP18; C17orf97, CAPN9, CCDC163P, DDX58, DDX60, ERAP2, IFIH1, KLF13, MICA, and ACOT1; C17orf97, CAPN9, CCDC163P, DDX58, DDX60, ERAP2, IFIH1, KLF13, MICA, and ARHGEF35; C17orf97, CAPN9, CCDC163P, DDX58, DDX60, ERAP2, IFIH1, KLF13, MICA, and NOMO3; C17orf97, CAPN9, CCDC163P, DDX58, DDX60, ERAP2, IFIH1, KLF13, MICA, and PAM; C17orf97, CAPN9, CCDC163P, DDX58, DDX60, ERAP2, IFIH1, KLF13, MICA, and THNSL2; C17orf97, CAPN9, CCDC163P, DDX58, DDX60, ERAP2, IFIH1, KLF13, MICA, and TMC4; C17orf97, CAPN9, CCDC163P, DDX58, DDX60, ERAP2, IFIH1, KLF13, MICA, and USP18; CAPN9, CCDC163P, DDX58, DDX60, ERAP2, IFIH1, KLF13, MICA, NOMO3, and ACOT1; CAPN9, CCDC163P, DDX58, DDX60, ERAP2, IFIH1, KLF13, MICA, NOMO3, and ARHGEF35; CAPN9, CCDC163P, DDX58, DDX60, ERAP2, IFIH1, KLF13, MICA, NOMO3, and C17orf97; CAPN9, CCDC163P, DDX58, DDX60, ERAP2, IFIH1, KLF13, MICA, NOMO3, and PAM; CAPN9, CCDC163P, DDX58, DDX60, ERAP2, IFIH1, KLF13, MICA, NOMO3, and THNSL2; CAPN9, CCDC163P, DDX58, DDX60, ERAP2, IFIH1, KLF13, MICA, NOMO3, and, TMC4; CAPN9, CCDC163P, DDX58, DDX60, ERAP2, IFIH1, KLF13, MICA, NOMO3, and USP18; CCDC163P, DDX58, DDX60, ERAP2, IFIH1, KLF13, MICA, NOMO3, PAM, and ACOT1; CCDC163P, DDX58, DDX60, ERAP2, IFIH1, KLF13, MICA, NOMO3, PAM, and ARHGEF35; CCDC163P, DDX58, DDX60, ERAP2, IFIH1, KLF13, MICA, NOMO3, PAM, and C17orf97; CCDC163P, DDX58, DDX60, ERAP2, IFIH1, KLF13, MICA, NOMO3, PAM, and CAPN9; CCDC163P, DDX58, DDX60, ERAP2, IFIH1, KLF13, MICA, NOMO3, PAM, and THNSL2; CCDC163P, DDX58, DDX60, ERAP2, IFIH1, KLF13, MICA, NOMO3, PAM, and TMC4; CCDC163P, DDX58, DDX60, ERAP2, IFIH1, KLF13, MICA, NOMO3, PAM, and USP18; DDX58, DDX60, ERAP2, IFIH1, KLF13, MICA, NOMO3, PAM, THNSL2, and ACOT1; DDX58, DDX60, ERAP2, IFIH1, KLF13, MICA, NOMO3, PAM, THNSL2, and ARHGEF35; DDX58, DDX60, ERAP2, IFIH1, KLF13, MICA, NOMO3, PAM, THNSL2, and C17orf97; DDX58, DDX60, ERAP2, IFIH1, KLF13, MICA, NOMO3, PAM, THNSL2, and CAPN9; DDX58, DDX60, ERAP2, IFIH1, KLF13, MICA, NOMO3, PAM, THNSL2, and CCDC163P; DDX58, DDX60, ERAP2, IFIH1, KLF13, MICA, NOMO3, PAM, THNSL2, and TMC4; DDX58, DDX60, ERAP2, IFIH1, KLF13, MICA, NOMO3, PAM, THNSL2, and USP18; DDX60, ERAP2, IFIH1, KLF13, MICA, NOMO3, PAM, THNSL2, TMC4, and ACOT1; DDX60, ERAP2, IFIH1, KLF13, MICA, NOMO3, PAM, THNSL2, TMC4, and ARHGEF35; DDX60, ERAP2, IFIH1, KLF13, MICA, NOMO3, PAM, THNSL2, TMC4, and C17orf97; DDX60, ERAP2, IFIH1, KLF13, MICA, NOMO3, PAM, THNSL2, TMC4, and CAPN9; DDX60, ERAP2, IFIH1, KLF13, MICA, NOMO3, PAM, THNSL2, TMC4, and CCDC163P; DDX60, ERAP2, IFIH1, KLF13, MICA, NOMO3, PAM, THNSL2, TMC4, and DDX58; DDX60, ERAP2, IFIH1, KLF13, MICA, NOMO3, PAM, THNSL2, TMC4, and USP18).

In some embodiments, the markers are 11 or more of the markers (e.g., ACOT1, ARHGEF35, C17orf97, CAPN9, CCDC163P, DDX58, DDX60, ERAP2, IFIH1, KLF13, and MICA; ACOT1, ARHGEF35, C17orf97, CAPN9, CCDC163P, DDX58, DDX60, ERAP2, IFIH1, KLF13, and NOMO3; ACOT1, ARHGEF35, C17orf97, CAPN9, CCDC163P, DDX58, DDX60, ERAP2, IFIH1, KLF13, and PAM; ACOT1, ARHGEF35, C17orf97, CAPN9, CCDC163P, DDX58, DDX60, ERAP2, IFIH1, KLF13, and THNSL2; ACOT1, ARHGEF35, C17orf97, CAPN9, CCDC163P, DDX58, DDX60, ERAP2, IFIH1, KLF13, and TMC4; ACOT1, ARHGEF35, C17orf97, CAPN9, CCDC163P, DDX58, DDX60, ERAP2, IFIH1, KLF13, and USP18; ARHGEF35, C17orf97, CAPN9, CCDC163P, DDX58, DDX60, ERAP2, IFIH1, KLF13, MICA, and ACOT1; ARHGEF35, C17orf97, CAPN9, CCDC163P, DDX58, DDX60, ERAP2, IFIH1, KLF13, MICA, and NOMO3; ARHGEF35, C17orf97, CAPN9, CCDC163P, DDX58, DDX60, ERAP2, IFIH1, KLF13, MICA, and PAM; ARHGEF35, C17orf97, CAPN9, CCDC163P, DDX58, DDX60, ERAP2, IFIH1, KLF13, MICA, and THNSL2; ARHGEF35, C17orf97, CAPN9, CCDC163P, DDX58, DDX60, ERAP2, IFIH1, KLF13, MICA, and TMC4; ARHGEF35, C17orf97, CAPN9, CCDC163P, DDX58, DDX60, ERAP2, IFIH1, KLF13, MICA, and USP18; C17orf97, CAPN9, CCDC163P, DDX58, DDX60, ERAP2, IFIH1, KLF13, MICA, NOMO3, and ACOT1; C17orf97, CAPN9, CCDC163P, DDX58, DDX60, ERAP2, IFIH1, KLF13, MICA, NOMO3, and ARHGEF35; C17orf97, CAPN9, CCDC163P, DDX58, DDX60, ERAP2, IFIH1, KLF13, MICA, NOMO3, and PAM; C17orf97, CAPN9, CCDC163P, DDX58, DDX60, ERAP2, IFIH1, KLF13, MICA, NOMO3, and THNSL2; C17orf97, CAPN9, CCDC163P, DDX58, DDX60, ERAP2, IFIH1, KLF13, MICA, NOMO3, and TMC4; C17orf97, CAPN9, CCDC163P, DDX58, DDX60, ERAP2, IFIH1, KLF13, MICA, NOMO3, and USP18; CAPN9, CCDC163P, DDX58, DDX60, ERAP2, IFIH1, KLF13, MICA, NOMO3, PAM, THNSL2, and ACOT1; CAPN9, CCDC163P, DDX58, DDX60, ERAP2, IFIH1, KLF13, MICA, NOMO3, PAM, THNSL2, and ARHGEF35; CAPN9, CCDC163P, DDX58, DDX60, ERAP2, IFIH1, KLF13, MICA, NOMO3, PAM, THNSL2, and C17orf97; CAPN9, CCDC163P, DDX58, DDX60, ERAP2, IFIH1, KLF13, MICA, NOMO3, PAM, THNSL2, and TMC4; CAPN9, CCDC163P, DDX58, DDX60, ERAP2, IFIH1, KLF13, MICA, NOMO3, PAM, THNSL2, and USP18; CCDC163P, DDX58, DDX60, ERAP2, IFIH1, KLF13, MICA, NOMO3, PAM, THNSL2, and ACOT1; CCDC163P, DDX58, DDX60, ERAP2, IFIH1, KLF13, MICA, NOMO3, PAM, THNSL2, and ARHGEF35; CCDC163P, DDX58, DDX60, ERAP2, IFIH1, KLF13, MICA, NOMO3, PAM, THNSL2, and C17orf97; CCDC163P, DDX58, DDX60, ERAP2, IFIH1, KLF13, MICA, NOMO3, PAM, THNSL2, and CAPN9; CCDC163P, DDX58, DDX60, ERAP2, IFIH1, KLF13, MICA, NOMO3, PAM, THNSL2, and TMC4; CCDC163P, DDX58, DDX60, ERAP2, IFIH1, KLF13, MICA, NOMO3, PAM, THNSL2, and USP18; DDX58, DDX60, ERAP2, IFIH1, KLF13, MICA, NOMO3, PAM, THNSL2, TMC4, and ACOT1; DDX58, DDX60, ERAP2, IFIH1, KLF13, MICA, NOMO3, PAM, THNSL2, TMC4, and ARHGEF35; DDX58, DDX60, ERAP2, IFIH1, KLF13, MICA, NOMO3, PAM, THNSL2, TMC4, and C17orf97; DDX58, DDX60, ERAP2, IFIH1, KLF13, MICA, NOMO3, PAM, THNSL2, TMC4, and CAPN9; DDX58, DDX60, ERAP2, IFIH1, KLF13, MICA, NOMO3, PAM, THNSL2, TMC4, and CCDC163P; DDX58, DDX60, ERAP2, IFIH1, KLF13, MICA, NOMO3, PAM, THNSL2, TMC4, and USP18).

In some embodiments, the markers are 12 or more of the markers (e.g., ACOT1, ARHGEF35, C17orf97, CAPN9, CCDC163P, DDX58, DDX60, ERAP2, IFIH1, KLF13, MICA, and NOMO3; ACOT1, ARHGEF35, C17orf97, CAPN9, CCDC163P, DDX58, DDX60, ERAP2, IFIH1, KLF13, MICA, and PAM; ACOT1, ARHGEF35, C17orf97, CAPN9, CCDC163P, DDX58, DDX60, ERAP2, IFIH1, KLF13, MICA, and THNSL2; ACOT1, ARHGEF35, C17orf97, CAPN9, CCDC163P, DDX58, DDX60, ERAP2, IFIH1, KLF13, MICA, and TMC4; ACOT1, ARHGEF35, C17orf97, CAPN9, CCDC163P, DDX58, DDX60, ERAP2, IFIH1, KLF13, MICA, and USP18; ARHGEF35, C17orf97, CAPN9, CCDC163P, DDX58, DDX60, ERAP2, IFIH1, KLF13, MICA, NOMO3, and ACOT1; ARHGEF35, C17orf97, CAPN9, CCDC163P, DDX58, DDX60, ERAP2, IFIH1, KLF13, MICA, NOMO3, and PAM; ARHGEF35, C17orf97, CAPN9, CCDC163P, DDX58, DDX60, ERAP2, IFIH1, KLF13, MICA, NOMO3, and THNSL2; ARHGEF35, C17orf97, CAPN9, CCDC163P, DDX58, DDX60, ERAP2, IFIH1, KLF13, MICA, NOMO3, and TMC4; ARHGEF35, C17orf97, CAPN9, CCDC163P, DDX58, DDX60, ERAP2, IFIH1, KLF13, MICA, NOMO3, and USP18; C17orf97, CAPN9, CCDC163P, DDX58, DDX60, ERAP2, IFIH1, KLF13, MICA, NOMO3, PAM, and ACOT1; C17orf97, CAPN9, CCDC163P, DDX58, DDX60, ERAP2, IFIH1, KLF13, MICA, NOMO3, PAM, and ARHGEF35; C17orf97, CAPN9, CCDC163P, DDX58, DDX60, ERAP2, IFIH1, KLF13, MICA, NOMO3, PAM, and THNSL2; C17orf97, CAPN9, CCDC163P, DDX58, DDX60, ERAP2, IFIH1, KLF13, MICA, NOMO3, PAM, and TMC4; C17orf97, CAPN9, CCDC163P, DDX58, DDX60, ERAP2, IFIH1, KLF13, MICA, NOMO3, PAM, and USP18; CAPN9, CCDC163P, DDX58, DDX60, ERAP2, IFIH1, KLF13, MICA, NOMO3, PAM, THNSL2, and ACOT1; CAPN9, CCDC163P, DDX58, DDX60, ERAP2, IFIH1, KLF13, MICA, NOMO3, PAM, THNSL2, and ARHGEF35; CAPN9, CCDC163P, DDX58, DDX60, ERAP2, IFIH1, KLF13, MICA, NOMO3, PAM, THNSL2, and C17orf97; CAPN9, CCDC163P, DDX58, DDX60, ERAP2, IFIH1, KLF13, MICA, NOMO3, PAM, THNSL2, and TMC4; CAPN9, CCDC163P, DDX58, DDX60, ERAP2, IFIH1, KLF13, MICA, NOMO3, PAM, THNSL2, and USP18; CCDC163P, DDX58, DDX60, ERAP2, IFIH1, KLF13, MICA, NOMO3, PAM, THNSL2, TMC4, and ACOT1; CCDC163P, DDX58, DDX60, ERAP2, IFIH1, KLF13, MICA, NOMO3, PAM, THNSL2, TMC4, and ARHGEF35; CCDC163P, DDX58, DDX60, ERAP2, IFIH1, KLF13, MICA, NOMO3, PAM, THNSL2, TMC4, and C17orf97; CCDC163P, DDX58, DDX60, ERAP2, IFIH1, KLF13, MICA, NOMO3, PAM, THNSL2, TMC4, and CAPN9; CCDC163P, DDX58, DDX60, ERAP2, IFIH1, KLF13, MICA, NOMO3, PAM, THNSL2, TMC4, and USP18).

In some embodiments, the markers are 13 or more of the markers (e.g., ACOT1, ARHGEF35, C17orf97, CAPN9, CCDC163P, DDX58, DDX60, ERAP2, IFIH1, KLF13, MICA, NOMO3, and PAM; ACOT1, ARHGEF35, C17orf97, CAPN9, CCDC163P, DDX58, DDX60, ERAP2, IFIH1, KLF13, MICA, NOMO3, and THNSL2; ACOT1, ARHGEF35, C17orf97, CAPN9, CCDC163P, DDX58, DDX60, ERAP2, IFIH1, KLF13, MICA, NOMO3, and TMC4; ACOT1, ARHGEF35, C17orf97, CAPN9, CCDC163P, DDX58, DDX60, ERAP2, IFIH1, KLF13, MICA, NOMO3, and USP18; ARHGEF35, C17orf97, CAPN9, CCDC163P, DDX58, DDX60, ERAP2, IFIH1, KLF13, MICA, NOMO3, PAM, and ACOT1; ARHGEF35, C17orf97, CAPN9, CCDC163P, DDX58, DDX60, ERAP2, IFIH1, KLF13, MICA, NOMO3, PAM, and THNSL2; ARHGEF35, C17orf97, CAPN9, CCDC163P, DDX58, DDX60, ERAP2, IFIH1, KLF13, MICA, NOMO3, PAM, and TMC4; ARHGEF35, C17orf97, CAPN9, CCDC163P, DDX58, DDX60, ERAP2, IFIH1, KLF13, MICA, NOMO3, PAM, and USP18; C17orf97, CAPN9, CCDC163P, DDX58, DDX60, ERAP2, IFIH1, KLF13, MICA, NOMO3, PAM, THNSL2, and ACOT1; C17orf97, CAPN9, CCDC163P, DDX58, DDX60, ERAP2, IFIH1, KLF13, MICA, NOMO3, PAM, THNSL2, and ARHGEF35; C17orf97, CAPN9, CCDC163P, DDX58, DDX60, ERAP2, IFIH1, KLF13, MICA, NOMO3, PAM, THNSL2, and TMC4; C17orf97, CAPN9, CCDC163P, DDX58, DDX60, ERAP2, IFIH1, KLF13, MICA, NOMO3, PAM, THNSL2, and USP18; CAPN9, CCDC163P, DDX58, DDX60, ERAP2, IFIH1, KLF13, MICA, NOMO3, PAM, THNSL2, TMC4, and ACOT1; CAPN9, CCDC163P, DDX58, DDX60, ERAP2, IFIH1, KLF13, MICA, NOMO3, PAM, THNSL2, TMC4, and ARHGEF35; CAPN9, CCDC163P, DDX58, DDX60, ERAP2, IFIH1, KLF13, MICA, NOMO3, PAM, THNSL2, TMC4, and C17orf97; CAPN9, CCDC163P, DDX58, DDX60, ERAP2, IFIH1, KLF13, MICA, NOMO3, PAM, THNSL2, TMC4, and USP18).

In some embodiments, the markers are 14 or more of the markers (e.g., ACOT1, ARHGEF35, C17orf97, CAPN9, CCDC163P, DDX58, DDX60, ERAP2, IFIH1, KLF13, MICA, NOMO3, PAM, and THNSL2; ACOT1, ARHGEF35, C17orf97, CAPN9, CCDC163P, DDX58, DDX60, ERAP2, IFIH1, KLF13, MICA, NOMO3, PAM, and TMC4; ACOT1, ARHGEF35, C17orf97, CAPN9, CCDC163P, DDX58, DDX60, ERAP2, IFIH1, KLF13, MICA, NOMO3, PAM, and USP18; ARHGEF35, C17orf97, CAPN9, CCDC163P, DDX58, DDX60, ERAP2, IFIH1, KLF13, MICA, NOMO3, PAM, THNSL2, and ACOT1; ARHGEF35, C17orf97, CAPN9, CCDC163P, DDX58, DDX60, ERAP2, IFIH1, KLF13, MICA, NOMO3, PAM, THNSL2, and TMC4; ARHGEF35, C17orf97, CAPN9, CCDC163P, DDX58, DDX60, ERAP2, IFIH1, KLF13, MICA, NOMO3, PAM, THNSL2, and USP18; C17orf97, CAPN9, CCDC163P, DDX58, DDX60, ERAP2, IFIH1, KLF13, MICA, NOMO3, PAM, THNSL2, TMC4, and ACOT1; C17orf97, CAPN9, CCDC163P, DDX58, DDX60, ERAP2, IFIH1, KLF13, MICA, NOMO3, PAM, THNSL2, TMC4, and ARHGEF35; C17orf97, CAPN9, CCDC163P, DDX58, DDX60, ERAP2, IFIH1, KLF13, MICA, NOMO3, PAM, THNSL2, TMC4, and USP18).

In some embodiments, the markers are 15 or more of the markers (e.g., ACOT1, ARHGEF35, C17orf97, CAPN9, CCDC163P, DDX58, DDX60, ERAP2, IFIH1, KLF13, MICA, NOMO3, PAM, THNSL2, TMC4, or USP18 ACOT1, ARHGEF35, C17orf97, CAPN9, CCDC163P, DDX58, DDX60, ERAP2, IFIH1, KLF13, MICA, NOMO3, PAM, THNSL2, and TMC4; ARHGEF35, C17orf97, CAPN9, CCDC163P, DDX58, DDX60, ERAP2, IFIH1, KLF13, MICA, NOMO3, PAM, THNSL2, TMC4, and USP18; ACOT1, C17orf97, CAPN9, CCDC163P, DDX58, DDX60, ERAP2, IFIH1, KLF13, MICA, NOMO3, PAM, THNSL2, TMC4, and USP18; ACOT1, ARHGEF35, CAPN9, CCDC163P, DDX58, DDX60, ERAP2, IFIH1, KLF13, MICA, NOMO3, PAM, THNSL2, TMC4, and USP18; ACOT1, ARHGEF35, C17orf97, CCDC163P, DDX58, DDX60, ERAP2, IFIH1, KLF13, MICA, NOMO3, PAM, THNSL2, TMC4, and USP18; ACOT1, ARHGEF35, C17orf97, CAPN9, DDX58, DDX60, ERAP2, IFIH1, KLF13, MICA, NOMO3, PAM, THNSL2, TMC4, and USP18; ACOT1, ARHGEF35, C17orf97, CAPN9, CCDC163P, DDX60, ERAP2, IFIH1, KLF13, MICA, NOMO3, PAM, THNSL2, TMC4, and USP18; ACOT1, ARHGEF35, C17orf97, CAPN9, CCDC163P, DDX58, ERAP2, IFIH1, KLF13, MICA, NOMO3, PAM, THNSL2, TMC4, and USP18; ACOT1, ARHGEF35, C17orf97, CAPN9, CCDC163P, DDX58, DDX60, IFIH1, KLF13, MICA, NOMO3, PAM, THNSL2, TMC4, and USP18; ACOT1, ARHGEF35, C17orf97, CAPN9, CCDC163P, DDX58, DDX60, ERAP2, KLF13, MICA, NOMO3, PAM, THNSL2, TMC4, and USP18; ACOT1, ARHGEF35, C17orf97, CAPN9, CCDC163P, DDX58, DDX60, ERAP2, IFIH1, MICA, NOMO3, PAM, THNSL2, TMC4, and USP18; ACOT1, ARHGEF35, C17orf97, CAPN9, CCDC163P, DDX58, DDX60, ERAP2, IFIH1, KLF13, NOMO3, PAM, THNSL2, TMC4, and USP18; ACOT1, ARHGEF35, C17orf97, CAPN9, CCDC163P, DDX58, DDX60, ERAP2, IFIH1, KLF13, MICA, PAM, THNSL2, TMC4, and USP18; ACOT1, ARHGEF35, C17orf97, CAPN9, CCDC163P, DDX58, DDX60, ERAP2, IFIH1, KLF13, MICA, NOMO3, THNSL2, TMC4, and USP18; ACOT1, ARHGEF35, C17orf97, CAPN9, CCDC163P, DDX58, DDX60, ERAP2, IFIH1, KLF13, MICA, NOMO3, PAM, TMC4, and USP18; ACOT1, ARHGEF35, C17orf97, CAPN9, CCDC163P, DDX58, DDX60, ERAP2, IFIH1, KLF13, MICA, NOMO3, PAM, THNSL2, and USP18).

The present invention is not limited to particular methods of detecting the level of the recited markers. Markers may be detected as DNA (e.g., cDNA), RNA (e.g., mRNA), or protein.

In some embodiments, nucleic acid sequencing methods are utilized for detection. In some embodiments, the technology provided herein finds use in a Second Generation (a.k.a. Next Generation or Next-Gen), Third Generation (a.k.a. Next-Next-Gen), or Fourth Generation (a.k.a. N3-Gen) sequencing technology including, but not limited to, pyrosequencing, sequencing-by-ligation, single molecule sequencing, sequence-by-synthesis (SBS), semiconductor sequencing, massive parallel clonal, massive parallel single molecule SBS, massive parallel single molecule real-time, massive parallel single molecule real-time nanopore technology, etc. Morozova and Marra provide a review of some such technologies in Genomics, 92:255 (2008), herein incorporated by reference in its entirety. Those of ordinary skill in the art will recognize that because RNA is less stable in the cell and more prone to nuclease attack experimentally RNA is usually reverse transcribed to DNA before sequencing.

A number of DNA sequencing techniques are suitable, including fluorescence-based sequencing methodologies (See, e.g., Birren et al., Genome Analysis: Analyzing DNA, 1, Cold Spring Harbor, N.Y.; herein incorporated by reference in its entirety). In some embodiments, the technology finds use in automated sequencing techniques understood in that art. In some embodiments, the present technology finds use in parallel sequencing of partitioned amplicons (PCT Publication No: WO2006084132 to Kevin McKernan et al., herein incorporated by reference in its entirety). In some embodiments, the technology finds use in DNA sequencing by parallel oligonucleotide extension (See, e.g., U.S. Pat. No. 5,750,341 to Macevicz et al., and U.S. Pat. No. 6,306,597 to Macevicz et al., both of which are herein incorporated by reference in their entireties). Additional examples of sequencing techniques in which the technology finds use include the Church polony technology (Mitra et al., 2003, Analytical Biochemistry 320, 55-65; Shendure et al., 2005 Science 309, 1728-1732; U.S. Pat. Nos. 6,432,360, 6,485,944, U.S. Pat. No. 6,511,803; herein incorporated by reference in their entireties), the 454 picotiter pyrosequencing technology (Margulies et al., 2005 Nature 437, 376-380; US20050130173; herein incorporated by reference in their entireties), the Solexa single base addition technology (Bennett et al., 2005, Pharmacogenomics, 6, 373-382; U.S. Pat. Nos. 6,787,308; 6,833,246; herein incorporated by reference in their entireties), the Lynx massively parallel signature sequencing technology (Brenner et al. (2000). Nat. Biotechnol. 18:630-634; U.S. Pat. Nos. 5,695,934; 5,714,330; herein incorporated by reference in their entireties), and the Adessi PCR colony technology (Adessi et al. (2000). Nucleic Acid Res. 28, E87; WO 00018957; herein incorporated by reference in its entirety).

Next-generation sequencing (NGS) methods share the common feature of massively parallel, high-throughput strategies, with the goal of lower costs in comparison to older sequencing methods (see, e.g., Voelkerding et al., Clinical Chem., 55:641-658, 2009; MacLean et al., Nature Rev. Microbiol., 7:287-296; each herein incorporated by reference in their entirety). NGS methods can be broadly divided into those that typically use template amplification and those that do not. Amplification-requiring methods include pyrosequencing commercialized by Roche as the 454 technology platforms (e.g., GS 20 and GS FLX), Life Technologies/Ion Torrent, the Solexa platform commercialized by Illumina, GnuBio, and the Supported Oligonucleotide Ligation and Detection (SOLID) platform commercialized by Applied Biosystems. Non-amplification approaches, also known as single-molecule sequencing, are exemplified by the HeliScope platform commercialized by Helicos BioSciences, and emerging platforms commercialized by VisiGen, Oxford Nanopore Technologies Ltd., and Pacific Biosciences, respectively.

In some embodiments, hybridization methods are utilized. Illustrative non-limiting examples of nucleic acid hybridization techniques include, but are not limited to, in situ hybridization (ISH), microarray, and Southern or Northern blot.

In situ hybridization (ISH) is a type of hybridization that uses a labeled complementary DNA or RNA strand as a probe to localize a specific DNA or RNA sequence in a portion or section of tissue (in situ), or, if the tissue is small enough, the entire tissue (whole mount ISH). DNA ISH can be used to determine the structure of chromosomes. RNA ISH is used to measure and localize mRNAs and other transcripts within tissue sections or whole mounts. Sample cells and tissues are usually treated to fix the target transcripts in place and to increase access of the probe. The probe hybridizes to the target sequence at elevated temperature, and then the excess probe is washed away. The probe that was labeled with radio-, fluorescent- or antigen-labeled bases is localized and quantitated in the tissue using autoradiography, fluorescence microscopy or immunohistochemistry. ISH can also use two or more probes, labeled with radioactivity or the other non-radioactive labels, to simultaneously detect two or more transcripts.

In some embodiments, markers are detected using fluorescence in situ hybridization (FISH). The preferred FISH assays for methods of embodiments of the present disclosure utilize bacterial artificial chromosomes (BACs). These have been used extensively in the human genome sequencing project (see Nature 409:953-958 (2001)) and clones containing specific BACs are available through distributors that can be located through many sources, e.g., NCBI. Each BAC clone from the human genome has been given a reference name that unambiguously identifies it. These names can be used to find a corresponding GenBank sequence and to order copies of the clone from a distributor.

Different kinds of biological assays are called microarrays including, but not limited to: microarrays (e.g., cDNA microarrays and oligonucleotide microarrays); protein microarrays; tissue microarrays; transfection or cell microarrays; chemical compound microarrays; and antibody microarrays. A DNA microarray, commonly known as gene chip, DNA chip, or biochip, is a collection of microscopic DNA spots attached to a solid surface (e.g., glass, plastic or silicon chip) forming an array for the purpose of expression profiling or monitoring expression levels for thousands of genes simultaneously. The affixed DNA segments are known as probes, thousands of which can be used in a single DNA microarray. Microarrays can be used to identify disease genes by comparing gene expression in disease and normal cells. Microarrays can be fabricated using a variety of technologies, including but not limited to: printing with fine-pointed pins onto glass slides; photolithography using pre-made masks; photolithography using dynamic micromirror devices; ink-jet printing; or, electrochemistry on microelectrode arrays.

Southern and Northern blotting may be used to detect specific DNA or RNA sequences, respectively. In these techniques DNA or RNA is extracted from a sample, fragmented, electrophoretically separated on a matrix gel, and transferred to a membrane filter. The filter bound DNA or RNA is subject to hybridization with a labeled probe complementary to the sequence of interest. Hybridized probe bound to the filter is detected. A variant of the procedure is the reverse Northern blot, in which the substrate nucleic acid that is affixed to the membrane is a collection of isolated DNA fragments and the probe is RNA extracted from a tissue and labeled.

In some embodiments, marker sequences are amplified (e.g., after conversion to DNA) prior to or simultaneous with detection. Illustrative non-limiting examples of nucleic acid amplification techniques include, but are not limited to, polymerase chain reaction (PCR), reverse transcription polymerase chain reaction (RT-PCR), transcription-mediated amplification (TMA), ligase chain reaction (LCR), strand displacement amplification (SDA), and nucleic acid sequence based amplification (NASBA). Those of ordinary skill in the art will recognize that certain amplification techniques (e.g., PCR) require that RNA be reversed transcribed to DNA prior to amplification (e.g., RT-PCR), whereas other amplification techniques directly amplify RNA (e.g., TMA and NASBA).

In some embodiments, quantitative evaluation of the amplification process in real-time is performed. Evaluation of an amplification process in “real-time” involves determining the amount of amplicon in the reaction mixture either continuously or periodically during the amplification reaction, and using the determined values to calculate the amount of target sequence initially present in the sample. A variety of methods for determining the amount of initial target sequence present in a sample based on real-time amplification are well known in the art. These include methods disclosed in U.S. Pat. Nos. 6,303,305 and 6,541,205, each of which is herein incorporated by reference in its entirety. Another method for determining the quantity of target sequence initially present in a sample, but which is not based on a real-time amplification, is disclosed in U.S. Pat. No. 5,710,029, herein incorporated by reference in its entirety.

Amplification products may be detected in real-time through the use of various self-hybridizing probes, most of which have a stem-loop structure. Such self-hybridizing probes are labeled so that they emit differently detectable signals, depending on whether the probes are in a self-hybridized state or an altered state through hybridization to a target sequence. By way of non-limiting example, “molecular torches” are a type of self-hybridizing probe that includes distinct regions of self-complementarity (referred to as “the target binding domain” and “the target closing domain”) which are connected by a joining region (e.g., non-nucleotide linker) and which hybridize to each other under predetermined hybridization assay conditions. In a preferred embodiment, molecular torches contain single-stranded base regions in the target binding domain that are from 1 to about 20 bases in length and are accessible for hybridization to a target sequence present in an amplification reaction under strand displacement conditions. Under strand displacement conditions, hybridization of the two complementary regions, which may be fully or partially complementary, of the molecular torch is favored, except in the presence of the target sequence, which will bind to the single-stranded region present in the target binding domain and displace all or a portion of the target closing domain. The target binding domain and the target closing domain of a molecular torch include a detectable label or a pair of interacting labels (e.g., luminescent/quencher) positioned so that a different signal is produced when the molecular torch is self-hybridized than when the molecular torch is hybridized to the target sequence, thereby permitting detection of probe: target duplexes in a test sample in the presence of unhybridized molecular torches. Molecular torches and a variety of types of interacting label pairs, including fluorescence resonance energy transfer (FRET) labels, are disclosed in, for example U.S. Pat. Nos. 6,534,274 and 5,776,782, each of which is herein incorporated by reference in its entirety.

The interaction between two molecules can also be detected, e.g., using fluorescence energy transfer (FRET) (see, for example, Lakowicz et al., U.S. Pat. No. 5,631,169; Stavrianopoulos et al., U.S. Pat. No. 4,968,103; each of which is herein incorporated by reference). A fluorophore label is selected such that a first donor molecule's emitted fluorescent energy will be absorbed by a fluorescent label on a second, ‘acceptor’ molecule, which in turn is able to fluoresce due to the absorbed energy.

Alternately, the ‘donor’ protein molecule may simply utilize the natural fluorescent energy of tryptophan residues. Labels are chosen that emit different wavelengths of light, such that the ‘acceptor’ molecule label may be differentiated from that of the ‘donor’. Since the efficiency of energy transfer between the labels is related to the distance separating the molecules, the spatial relationship between the molecules can be assessed. In a situation in which binding occurs between the molecules, the fluorescent emission of the ‘acceptor’ molecule label should be maximal. A FRET binding event can be conveniently measured through standard fluorometric detection means well known in the art (e.g., using a fluorimeter).

Another example of a detection probe having self-complementarity is a “molecular beacon.” Molecular beacons include nucleic acid molecules having a target complementary sequence, an affinity pair (or nucleic acid arms) holding the probe in a closed conformation in the absence of a target sequence present in an amplification reaction, and a label pair that interacts when the probe is in a closed conformation. Hybridization of the target sequence and the target complementary sequence separates the members of the affinity pair, thereby shifting the probe to an open conformation. The shift to the open conformation is detectable due to reduced interaction of the label pair, which may be, for example, a fluorophore and a quencher (e.g., DABCYL and EDANS). Molecular beacons are disclosed, for example, in U.S. Pat. Nos. 5,925,517 and 6,150,097, herein incorporated by reference in its entirety.

The cancer marker genes described herein may be detected as proteins using a variety of protein techniques known to those of ordinary skill in the art, including but not limited to, protein sequencing and immunoassays.

Illustrative non-limiting examples of protein sequencing techniques include, but are not limited to, mass spectrometry and Edman degradation.

Mass spectrometry can, in principle, sequence any size protein but becomes computationally more difficult as size increases. A protein is digested by an endoprotease, and the resulting solution is passed through a high pressure liquid chromatography column. At the end of this column, the solution is sprayed out of a narrow nozzle charged to a high positive potential into the mass spectrometer. The charge on the droplets causes them to fragment until only single ions remain. The peptides are then fragmented and the mass-charge ratios of the fragments measured. The mass spectrum is analyzed by computer and often compared against a database of previously sequenced proteins in order to determine the sequences of the fragments. The process is then repeated with a different digestion enzyme, and the overlaps in sequences are used to construct a sequence for the protein.

In the Edman degradation reaction, the peptide to be sequenced is adsorbed onto a solid surface (e.g., a glass fiber coated with polybrene). The Edman reagent, phenylisothiocyanate (PTC), is added to the adsorbed peptide, together with a mildly basic buffer solution of 12% trimethylamine, and reacts with the amine group of the N-terminal amino acid. The terminal amino acid derivative can then be selectively detached by the addition of anhydrous acid. The derivative isomerizes to give a substituted phenylthiohydantoin, which can be washed off and identified by chromatography, and the cycle can be repeated. The efficiency of each step is about 98%, which allows about 50 amino acids to be reliably determined.

Illustrative non-limiting examples of immunoassays include, but are not limited to: immunoprecipitation; Western blot; ELISA; immunohistochemistry; immunocytochemistry; flow cytometry; and immuno-PCR. Polyclonal or monoclonal antibodies detectably labeled using various techniques known to those of ordinary skill in the art (e.g., colorimetric, fluorescent, chemiluminescent or radioactive) are suitable for use in the immunoassays. Immunoprecipitation is the technique of precipitating an antigen out of solution using an antibody specific to that antigen. The process can be used to identify protein complexes present in cell extracts by targeting a protein believed to be in the complex. The complexes are brought out of solution by insoluble antibody-binding proteins isolated initially from bacteria, such as Protein A and Protein G. The antibodies can also be coupled to sepharose beads that can easily be isolated out of solution. After washing, the precipitate can be analyzed using mass spectrometry, Western blotting, or any number of other methods for identifying constituents in the complex.

A Western blot, or immunoblot, is a method to detect protein in a given sample of tissue homogenate or extract. It uses gel electrophoresis to separate denatured proteins by mass. The proteins are then transferred out of the gel and onto a membrane, typically polyvinyldiflroride or nitrocellulose, where they are probed using antibodies specific to the protein of interest. As a result, researchers can examine the amount of protein in a given sample and compare levels between several groups.

An ELISA, short for Enzyme-Linked ImmunoSorbent Assay, is a biochemical technique to detect the presence of an antibody or an antigen in a sample. It utilizes a minimum of two antibodies, one of which is specific to the antigen and the other of which is coupled to an enzyme. The second antibody will cause a chromogenic or fluorogenic substrate to produce a signal. Variations of ELISA include sandwich ELISA, competitive ELISA, and ELISPOT. Because the ELISA can be performed to evaluate either the presence of antigen or the presence of antibody in a sample, it is a useful tool both for determining serum antibody concentrations and also for detecting the presence of antigen.

Immunohistochemistry and immunocytochemistry refer to the process of localizing proteins in a tissue section or cell, respectively, via the principle of antigens in tissue or cells binding to their respective antibodies. Visualization is enabled by tagging the antibody with color producing or fluorescent tags. Typical examples of color tags include, but are not limited to, horseradish peroxidase and alkaline phosphatase. Typical examples of fluorophore tags include, but are not limited to, fluorescein isothiocyanate (FITC) or phycoerythrin (PE).

Flow cytometry is a technique for counting, examining and sorting microscopic particles suspended in a stream of fluid. It allows simultaneous multiparametric analysis of the physical and/or chemical characteristics of single cells flowing through an optical/electronic detection apparatus. A beam of light (e.g., a laser) of a single frequency or color is directed onto a hydrodynamically focused stream of fluid. A number of detectors are aimed at the point where the stream passes through the light beam; one in line with the light beam (Forward Scatter or FSC) and several perpendicular to it (Side Scatter (SSC) and one or more fluorescent detectors). Each suspended particle passing through the beam scatters the light in some way, and fluorescent chemicals in the particle may be excited into emitting light at a lower frequency than the light source. The combination of scattered and fluorescent light is picked up by the detectors, and by analyzing fluctuations in brightness at each detector, one for each fluorescent emission peak, it is possible to deduce various facts about the physical and chemical structure of each individual particle. FSC correlates with the cell volume and SSC correlates with the density or inner complexity of the particle (e.g., shape of the nucleus, the amount and type of cytoplasmic granules or the membrane roughness).

Immuno-polymerase chain reaction (IPCR) utilizes nucleic acid amplification techniques to increase signal generation in antibody-based immunoassays. Because no protein equivalence of PCR exists, that is, proteins cannot be replicated in the same manner that nucleic acid is replicated during PCR, the only way to increase detection sensitivity is by signal amplification. The target proteins are bound to antibodies which are directly or indirectly conjugated to oligonucleotides. Unbound antibodies are washed away and the remaining bound antibodies have their oligonucleotides amplified. Protein detection occurs via detection of amplified oligonucleotides using standard nucleic acid detection methods, including real-time methods. Embodiments of the present invention further provide kits and systems comprising reagents for detection of the recited markers (e.g., primer, probes, etc.). In some embodiments, kits and systems comprise computer systems for analyzing marker levels and providing diagnoses, prognoses, or determining treatment courses of action.

In some embodiments, a computer-based analysis program is used to translate the raw data generated by the detection assay (e.g., levels of the recited markers) into data of predictive value for a clinician. The clinician can access the predictive data using any suitable means. Thus, in some preferred embodiments, the present invention provides the further benefit that the clinician, who is not likely to be trained in genetics or molecular biology, need not understand the raw data. The data is presented directly to the clinician in its most useful form. The clinician is then able to immediately utilize the information in order to optimize the care of the subject.

The present invention contemplates any method capable of receiving, processing, and transmitting the information to and from laboratories conducting the assays, information provides, medical personal, and subjects. For example, in some embodiments of the present invention, a sample (e.g., a biopsy or a serum or urine sample) is obtained from a subject and submitted to a profiling service (e.g., clinical lab at a medical facility, genomic profiling business, etc.), located in any part of the world (e.g., in a country different than the country where the subject resides or where the information is ultimately used) to generate raw data. Where the sample comprises a tissue or other biological sample, the subject may visit a medical center to have the sample obtained and sent to the profiling center, or subjects may collect the sample themselves (e.g., a urine sample) and directly send it to a profiling center. Where the sample comprises previously determined biological information, the information may be directly sent to the profiling service by the subject (e.g., an information card containing the information may be scanned by a computer and the data transmitted to a computer of the profiling center using an electronic communication system). Once received by the profiling service, the sample is processed and a profile is produced (i.e., marker levels) specific for the diagnostic or prognostic information desired for the subject.

The profile data is then prepared in a format suitable for interpretation by a treating clinician. For example, rather than providing raw data, the prepared format may represent a diagnosis or risk assessment (e.g., level of markers) for the subject, along with recommendations for particular treatment options. The data may be displayed to the clinician by any suitable method. For example, in some embodiments, the profiling service generates a report that can be printed for the clinician (e.g., at the point of care) or displayed to the clinician on a computer monitor.

In some embodiments, the information is first analyzed at the point of care or at a regional facility. The raw data is then sent to a central processing facility for further analysis and/or to convert the raw data to information useful for a clinician or patient. The central processing facility provides the advantage of privacy (all data is stored in a central facility with uniform security protocols), speed, and uniformity of data analysis. The central processing facility can then control the fate of the data following treatment of the subject. For example, using an electronic communication system, the central facility can provide data to the clinician, the subject, or researchers.

In some embodiments, the subject is able to directly access the data using the electronic communication system. The subject may choose further intervention or counseling based on the results. In some embodiments, the data is used for research. For example, the data may be used to further optimize the inclusion or elimination of markers as useful indicators of a particular condition or stage of disease or as a companion diagnostic to determine a treatment course of action.

The compositions, kits, systems, uses, and methods described herein find use in the diagnosis and prognosis of prostate cancer, as well as in determining a treatment course of action for a subject diagnosed with prostate cancer. For example, in some embodiments, compositions and method described herein are used to provide a prognosis of one or more of risk of prostate cancer recurrence, risk of prostate cancer metastasis, and/or risk of prostate cancer-specific death. In some embodiments, such prognoses, along with marker levels, are used to determine a treatment course of action in a subject diagnosed with prostate cancer (e.g., surgery, use of adjuvant radiotherapy and/or androgen depletion therapy, or watchful waiting). In some embodiments, markers levels (e.g., in a prostate cancer biopsy, urine sample, blood sample, or bone marrow sample) are tested one or more times before, during, or after prostate cancer treatment. In some embodiments, marker levels are used to alter a prostate cancer treatment course of action (e.g., stop, start, or change a treatment).

In some preferred embodiments, subjects with an altered level of expression of one or more of the genes are identified as being at an increased risk for biological recurrence of prostate cancer (BCR). In some preferred embodiments, subjects with an altered level of expression of one or more of the genes are identified as being at an increased risk for biological recurrence of prostate cancer (BCR) as compared to subjects not having an altered level of expression of one or more the genes. In some preferred embodiments, the subjects have been previously diagnosed with prostate cancer. In some preferred embodiments, the subjects have undergone a radical prostatectomy.

In some preferred embodiments, the detection of an altered level of expression of one or more of the genes is used to stratify the subject into a risk group, e.g., a high risk group for BCR. In some preferred embodiments, the subject has already been identified as having a high or intermediate risk of BCR, and the detection of an altered level of expression of one or more of the genes is used to further stratify the subject into an additional risk group for BCR.

In some preferred embodiments, subjects exhibiting an altered level of expression of one or more of the genes are chosen for administration of an adjuvant treatment. In particularly preferred embodiments, the adjuvant treatment is administered after a radical prostatectomy. In some preferred embodiments, the adjuvant treatment is androgen deprivation therapy (ADT, also called hormonal therapy), chemotherapy, or a combination of ADT and chemotherapy.

The present invention is not limited to the use of any particular ADT. ADT includes administration of medications that stop production of testosterone by the body and medications that block testosterone from reaching cancer cells. Medications known as luteinizing hormone-releasing hormone (LHRH) or gonadotropin-releasing hormone (GnRH) agonists and antagonists prevent the body's cells from receiving messages to make testosterone. These medications are generally administered as shots every three to six months or via an implant. Suitable LNRH agonist and antagonist medications include, but are not limited to, Leuprolide (Eligard, Lupron Depot, etc.), Goserelin (Zoladex), Triptorelin (Trelstar), Histrelin (Vantas), and Degarelix (Firmagon). Anti-androgens block testosterone from reaching cancer cells. In some preferred embodiments, these oral medications are utilized with an LHRH agonist or before taking an LHRH agonist. Suitable anti-androgens include, but are not limited to, Bicalutamide (Casodex), Nilutamide (Nilandron), and Flutamide.

The present invention is not limited to the use of any particular chemotherapeutic agent. Suitable chemotherapeutic agents include, but are not limited to, Docetaxel (Taxotere), Cabazitaxel (Jevtana), Mitoxantrone (Novantrone) and Estramustine (Emcyt).

### EXPERIMENTAL

The following examples are provided in order to demonstrate and further illustrate certain preferred embodiments and aspects of the present invention and are not to be construed as limiting the scope thereof.

### Example 1

### Methods

### Patients and Tissue Samples

From a prospectively recruited cohort comprising 571 prostate cancer patients treated by radical prostatectomy between 2010 and 2012, three to seven malignant samples from each prostate were biobanked as fresh frozen tissue. From these, a subset of 22 patients were selected to enrich for patients where one benign and two to three malignant samples were available from at least two clearly separated foci (at least 2 mm separation and with distinct tissue morphology), in addition to one patient with two samples from a single focus. In total, 87 tissue samples (64 malignant and 23 benign samples) were selected for analysis. Fifteen patients have more than one sample from the same focus, and are eligible for analyses on intrafocal heterogeneity, while 22 patients have samples from more than one focus and are eligible for analyses on interfocal heterogeneity. For each malignant sample, the surrounding area was re-evaluated to determine the Gleason score and grade group according to the 2014 International Society of Urological Pathology (ISUP) modified Gleason grading system (14). In addition, it was determined whether each sample was in, or in close proximity to, an area with extraprostatic extension (EPE) and whether there was any benign tissue present. All samples have previously been analyzed by deep coverage whole-exome sequencing (3). Written informed consent was obtained from all included patients and the study has been approved by the Regional Ethics Committee South-Eastern Norway (number 2013/595/REK southeast A).

### High-Throughput RNA Sequencing

The DNA and RNA were isolated as described previously (3). The total RNA (1.5 μg) derived from malignant (n=64) and benign (n=23) tissue was subjected to ribosomal RNA depletion and RNA sequencing libraries were prepared using the TruSeq Stranded Total RNA Library Prep Kit (Illumina, San Diego, CA), in accordance with the manufacturer's protocol. The libraries were sequenced using the Illumina HiSeq 2500 (n=40, in 2017) and the Illumina HiSeq 4000 (n=47, in 2019) at the Oslo University Hospital Genomics Core Facility. Both batches were sequenced with 2×101 basepairs paired-end configuration.

### Processing of Raw Sequence Data

Data was analyzed within the high-performance computational infrastructure of University of Oslo's Services for Sensitive Data. The RNA from the 87 malignant and benign samples were sequenced with an average depth of 116.8 million reads per sample. Generated FASTQ files were trimmed for adapter contamination and low base calling quality using

Trimmomatic (15) version 0.38. Quality of sequence reads was assessed using the FastQC software (16) and aggregated using the MultiQC software (17) (Trimmed reads were aligned to the human reference genome (GRCh38) with GENCODE (release 28) feature annotations, with an average of 44 million uniquely mapped read pairs per sample. Gene counts were quantified using HTSeq (18) version 0.10.0. These counts were normalized as transcripts per million (TPM) and corrected for batch effects using the removeBatchEffects ( ) function from the limma R package (19) (version 3.40.6). Gene expression values were compared between two technical replicates, one from each batch of sequencing. For downstream analyses, only long non-coding RNA (lncRNAs) and protein-coding genes with at least 10 raw read counts in at least 3 out of the 87 samples were included. From a total set of 27391 lncRNAs and protein-coding genes, 18349 remained after filtering.

### Fusion Gene Detection

DeFuse (20) (version 0.7.0), FusionCatcher (21) (version 1.20) and STAR-Fusion (22) (version 1.9.1) were applied for identification of fusion genes. Only fusion genes nominated by at least two out of the three algorithms were considered for further analysis. Fusion candidates where both partner genes were located on the same chromosome and less than 0.5 Mb apart were omitted from further analyses as they may be the result of RNA polymerase read-through events. We have focused on gene partners that are commonly recurring fusion genes in prostate cancer or otherwise relevant for the disease (23), including ERG, ETV1, ETV4, ETV5, FLI1, ELK4, BRAF, FGFR2, HPR, KRAS, RAF1, SCHLAP1, AMACR, AR, and MYC.

### Mutation Expression Analysis

Somatic point mutations and short insertions and deletions (indels) had previously been identified from exome sequencing data of the same set of samples (3). Read coverage of both mutated (variant) and wildtype (reference) alleles were quantified from RNA sequencing data using the tool ASEReadCounter from the Genome Analysis Toolkit (24) (version 3.8.1.0). Minimum read mapping quality was set to 10 and minimum base calling quality was set to 2. Read coverage for indels was derived from alignment data using the mpileup software from SAMtools (25) (version 1.8). Mutations were categorized as expressed if the mutated allele frequency (the fraction of reads originating from the mutated allele) was ≥5% (mutated allele expressed), and non-expressed if either the mutated allele frequency was <5% (mutated allele not expressed) or the TPM value of the mutated gene was <1.0 (mutated gene not expressed).

### Heterogeneity Scores

### Coefficient of Variation Scores

Intrapatient heterogeneity scores for genes were calculated per patient as the coefficient of variation of TPM expression values from all malignant samples from the same patient. A single score for each gene was calculated as the mean of intrapatient heterogeneity scores for that gene from all patients. Interpatient heterogeneity scores for genes were calculated as the coefficient of variation of TPM expression values in a set of malignant samples comprising one sample for each patient. Given the presence of multiple samples for each patient, the single sample representing each patient was selected at random. This random selection was performed ten times, and for each gene, the average coefficient of variation from the ten iterations represents its interpatient heterogeneity score.

### Intraclass Correlation Coefficient

Heterogeneity of gene expression was also quantified in terms of intraclass correlation coefficients (ICC). For each gene, a random effects model was generated using the lmer( ) function provided by the lme4 R-package (26) (version 1.1-23), with gene expression values serving as the response, and patient IDs representing random effects. Only malignant samples were used in generating the models and batch corrected TPM values were used as gene expression values. The icc( ) function provided by the performance (27) R package (version 0.5.0) was used to generate ICC values for each gene. Genes were categorized into four groups according to ICC value. Genes with ICC below 0.5 were categorized as “poor”, genes with ICC between 0.5 and 0.75 were categorized as “moderate”, genes with ICC between 0.75 and 0.90 were categorized as “good”, and genes with ICC greater than 0.9 were categorized as “excellent”. Genes with insufficient variation to calculate ICC were not scored. The ICC value of a gene, ranging from 0 to 1, represents the fraction of the total amount of variance in expression that is attributable to differences between patients, rather than within patients. A value of 1 means that 100% of the observed variance in expression among all samples is attributable to differences between patients, indicating high interpatient heterogeneity, and low intrapatient heterogeneity.

Association with Biochemical Recurrence

Hazard ratios and p-values from Cox proportional hazards regression analysis on biochemical recurrence-free survival were derived from expression analyses of 499 patients sourced from Table S3 in Kremer et al. (28). In that study, univariate and multivariate survival analyses were performed on patient groups dichotomized by gene expression. Two approaches were used for patient stratification, one based on median mRNA expression, the other an optimized approach considering all stratification levels and yielding the most performant one. The resource provides univariate analysis results for 17681 genes and multivariate analysis results for 9289 genes. Here, we select clinically relevant genes based on p-values from the univariate analysis where the optimized dichotomization strategy was used (uni_p_best).

### Selection of Clinically Relevant Genes

Genes with low intrapatient and high interpatient heterogeneity were selected if they had below average intrapatient heterogeneity and above average interpatient heterogeneity scores and were categorized in the “good” or “excellent” ICC categories. Further, clinically relevant genes were required to have a p-value less than 0.05 in univariate Cox regression, and a 25th percentile TPM value greater than 1. 3656 genes with missing hazard ratios and p-values were excluded as candidates for clinically relevant genes.

### Within-Patient Paired Samples Differential Gene Expression Analyses

Differential gene expression analysis (DGEA) was performed using the DESeq2 R package (29) (version 1.24.0), utilizing a paired samples experiment design to enable expression analyses with reference samples from within the same patient and prostate. Raw read counts from HTSeq were provided as input. Fold changes (FC) were shrunken using the lfcshrink( ) function in DESeq2 by the adaptive prior shrinkage estimator provided by the apeglm R package (30) (version 1.6.0). P-values were adjusted for multiple testing by independent hypothesis weighting provided by the IHW R package (31) (version 1.12.0), weighted by the average expression of each gene across all samples. Genes with absolute shrunken log₂ fold changes greater than 1 and adjusted p-values lower than 0.05 were deemed significantly differentially expressed.

### Gene Set Enrichment Analysis

Gene set enrichment analysis (GSEA) (32) was performed using the R packages gage (33) (version 2.34.0) and fgsea (34) (version 1.10.1). The Hallmarks and Biological Processes gene sets were downloaded from the Molecular Signatures Database (MSigDB) (35) release 7.0. Prior to analysis, gene symbols were mapped between GENCODE version 28 and GENCODE version 30 by ENSEMBL stable gene IDs. Genes with IDs with no corresponding symbol were omitted. Normalized enrichment scores were calculated on the output of DGEA, where genes were ranked by the Wald statistic produced by DESeq2. Gene sets were deemed significantly differentially regulated if both algorithms identified differential regulation in the same direction, and both reported an adjusted p-value of less than 0.05.

### ETS Classification

Malignant samples were classified as ETS positive or ETS negative based on the gene expression levels of ERG, ETV1, ETV4, and FLI1. Samples with high expression of one or more of the four genes were classified as ETS positive, samples with low expression in all four genes were classified as ETS negative. High or low expression levels for each gene were determined by the expectation-maximization algorithm, implemented in the R package mixtools (36) (version 1.2.0). Expression levels for ERG, ETV1, ETV4, and FLI1 were validated using real-time reverse transcription polymerase chain reaction (RT-PCR) with TaqMan Gene Expression Assays (Thermo Fisher Scientific). ABL1 was used as endogenous control. The applied assays were Hs01554630_ml (ERG), Hs00231877_ml (ETV1), Hs00944562_ml (ETV4), Hs00956709_ml (FLI1) and Hs01104728_ml (ABL1). All samples were run in triplicate on an ABI 7900HT Fast Real-time PCR system (Applied Biosystems, Foster City, CA), with 10 ng cDNA included in each reaction.

### Results

Transcriptome Sequencing of Multiple Foci from Individual Prostates

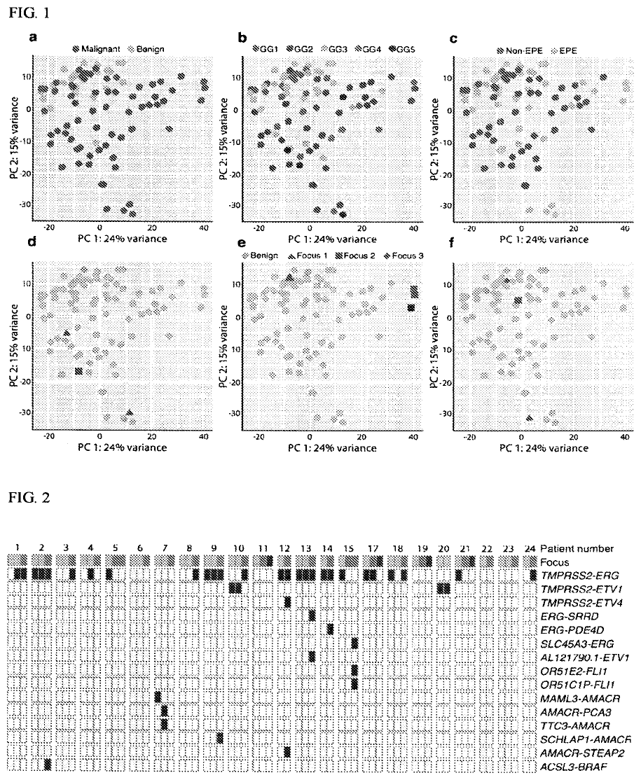

For a general overview of the prostate cancer transcriptomes, we performed principal component analysis (PCA) on the expression values of the 500 most varying genes and lncRNAs (FIG. 1). We observe that benign tissue samples cluster closely together (FIG. 1a), which suggests that much of the transcriptomic variation is explained by the presence of malignant cells inside the tumor focus. There is, however, a considerable presence of malignant samples within the cluster of benign samples, most of which are classified as grade group 1 or 2 (FIG. 1b). Further, we see that most of these malignant samples are of the less aggressive non-EPE phenotype, and that EPE samples cluster farther away from the samples with benign tissue and the samples with grade group 1 or 2 (FIG. 1c). We found that samples from different foci from the same patients have great variations in gene expression, which illustrates the heterogeneous nature of multifocal prostate cancer (selected patients in FIG. 1d-f).

### Inter- and Intrafocal Heterogeneity

### Heterogeneity in Expressed Fusion Transcripts

From the malignant samples, we identified 314 fusion events (average 4.9 per sample) in total. The TMPRSS2-ERG fusion gene was the most commonly expressed (27/64 malignant samples, 42.2%), constituting 20.7% of the expressed fusion events and represented by 18 different fusion breakpoints. Out of a total of 23 patients, 11 display interfocal heterogeneity of TMPRSS2-ERG expression (FIG. 2, patients 1, 3, 4, 5, 8, 10, 15, 17, 18, 21, and 24) and three display intrafocal heterogeneity of TMPRSS2-ERG expression (FIG. 2, patients 1, 4, and 18). In addition, fusion genes with an ETS partner gene other than ERG were identified in altogether 7 samples from 5 patients, and in 4 of them present in only one malignant focus per patient. Five patients showed no expression of prostate cancer relevant fusion genes, suggesting that 78% of all prostate cancers express fusion genes.

### Heterogeneity in Expressed Mutations

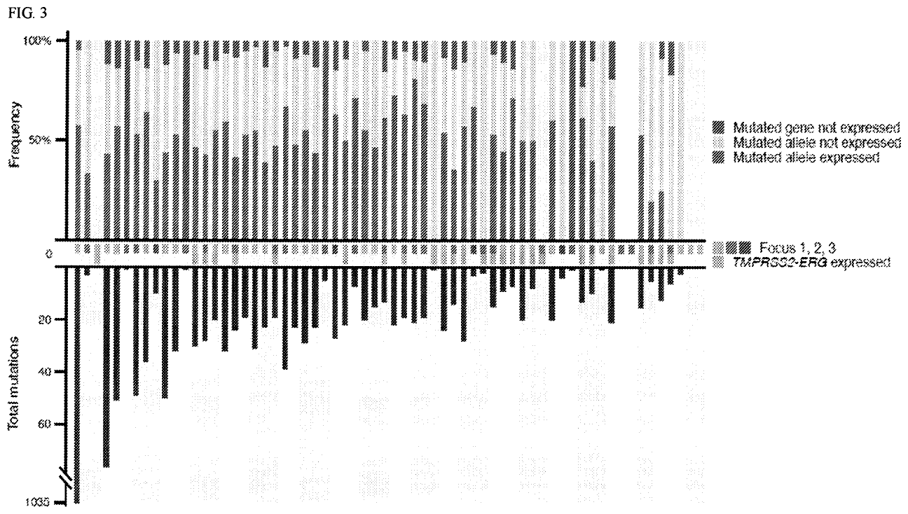

From a total of 1983 somatic and exonic mutations in the 64 malignant samples, 1084 (54.7%) were identified as expressed mutations, 762 (38.4%) were identified as not expressed mutations (preferential expression of wildtype allele) and 137 (6.9%) were identified as a non-expressed gene (FIG. 3). Out of the 1084 expressed mutations, 807 were eligible for calculating intrafocal heterogeneity, as they are expressed in samples representing foci from which we have multiple samples from the same patient. Among these, 731 (90.6%) are expressed only in a single sample from the patient, while 76 (9.4%) are expressed in multiple samples from the same focus. This indicates a high intrafocal heterogeneity of expressed somatic mutations. Additionally, we observe 100% interfocal heterogeneity, as none of the 1084 mutations are expressed across multiple different foci from the same patient.

### Heterogeneity in Gene Expression

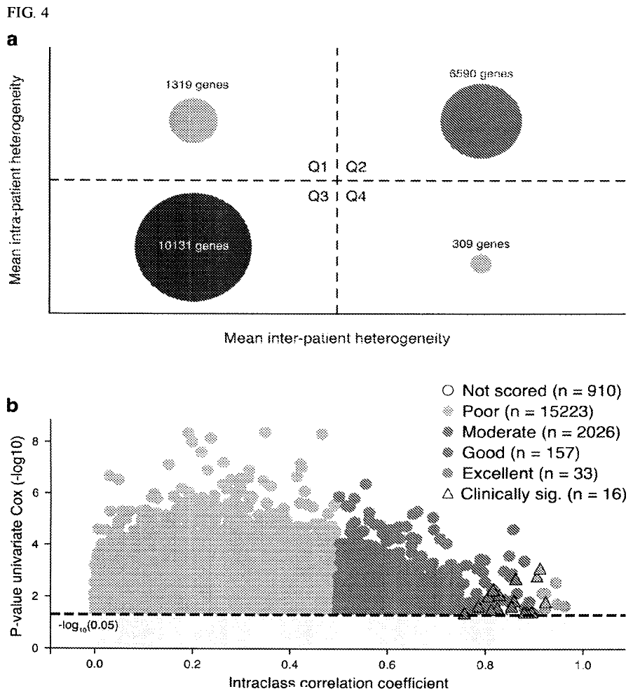

As demonstrated in the PCA plots above (FIG. 1), the transcriptome-wide gene-level expression pattern is in general highly variable among malignant foci. The variation is prominent whether we compare samples from the same patient or across patients (Pearson's r=0.83, intrapatient heterogeneity score vs. interpatient heterogeneity score for every gene). However, the multiple samples per patient enable a transcriptome-wide discovery of genes with biomarker properties at the patient level. 309 genes were nominated as those having lower than average intrapatient heterogeneity scores and higher than average interpatient scores (Q4, FIG. 4a). Further, 190 genes were identified as those with “good” or “excellent” ICC scores (FIG. 4b). The intersection of the two selections comprises 62 genes, 24 of which have statistically significant association with biochemical recurrence according to an external dataset (28). Finally, 16 of those genes have adequate expression levels across all samples and are thus nominated as promising prognostic biomarkers for primary prostate cancer, independent of which malignant focus the expression measurement is taken from (Table 1).

Differential Expression Between Paired Samples from Individual Prostates

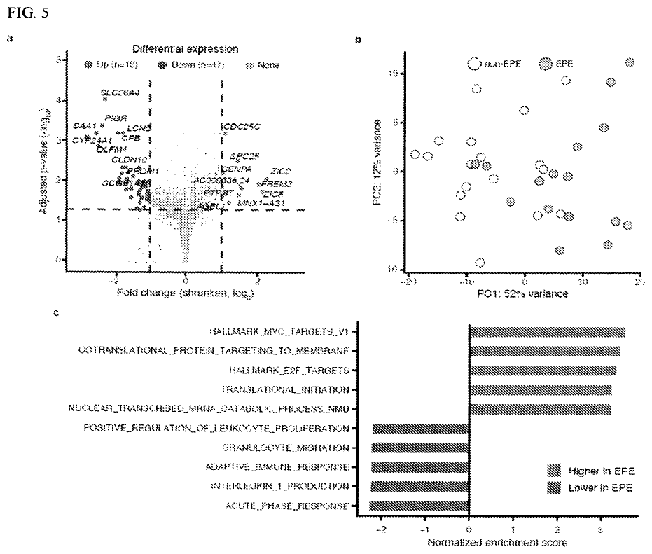

To identify gene expression associated with EPE, we performed paired-sampling analyses within each of 12 patients from whom we had at least one malignant sample with and without proximity to EPE, comprising altogether 35 samples. Among all genes analyzed, 13 genes were significantly higher expressed in EPE samples (log 2FC>1, adjusted p-values range from 6×10⁻⁴ to 0.03). Another 47 genes had significantly lower expression in EPE samples (log 2FC from −1.00 to −2.74, adjusted p-value ranges from 9×10⁻⁵ to 0.05). GSEA demonstrated MYC targets to be significantly enriched among genes upregulated in EPE, while immune responses seem to be downregulated (FIG. 5).

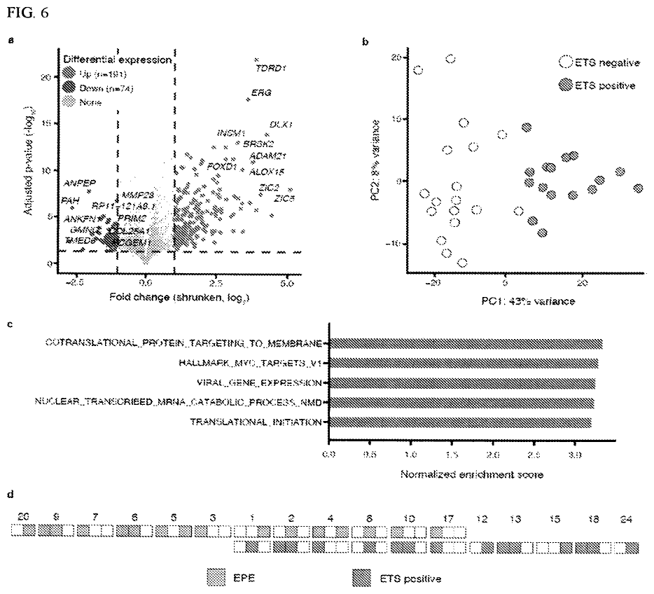

Similar within-patient pair-wise analyses were performed for ETS positive vs. ETS negative malignant samples from within the same prostates. Among 64 malignant samples, 38 were classified as ETS negative, while 26 were classified as ETS positive. Thirty-four samples were eligible for paired samples analyses from 12 patients with both ETS positive and ETS negative samples. Altogether, 191 genes were significantly higher expressed in ETS positive malignant tissue samples compared to ETS negative samples (log 2FC>1, adjusted p-value range from 2×10⁻²² to 0.025). Nineteen of these showed profound upregulation with log 2FC>3 (maximum log₂FC 5.1; adjusted p-values from 2×10⁻²² to 3×10⁻⁴). Another 74 genes were significantly downregulated in ETS-positive malignant tissue samples (log 2FC from −1 to −2.7; adjusted p-values range from 2×10⁻⁸ to 0.046). GSEA demonstrated that MYC targets were also for this comparison significantly enriched among genes upregulated in ETS positive malignant tissue samples, while there were no significantly downregulated gene sets (FIG. 6). The similar results from GSEA between EPE and non-EPE and ETS positive and ETS negative was not caused by the same malignant samples being positive in both analyses (X² (1, N=64)=2.16, p>0.1, FIG. 6d).

### Example 2

### Gene Rankings

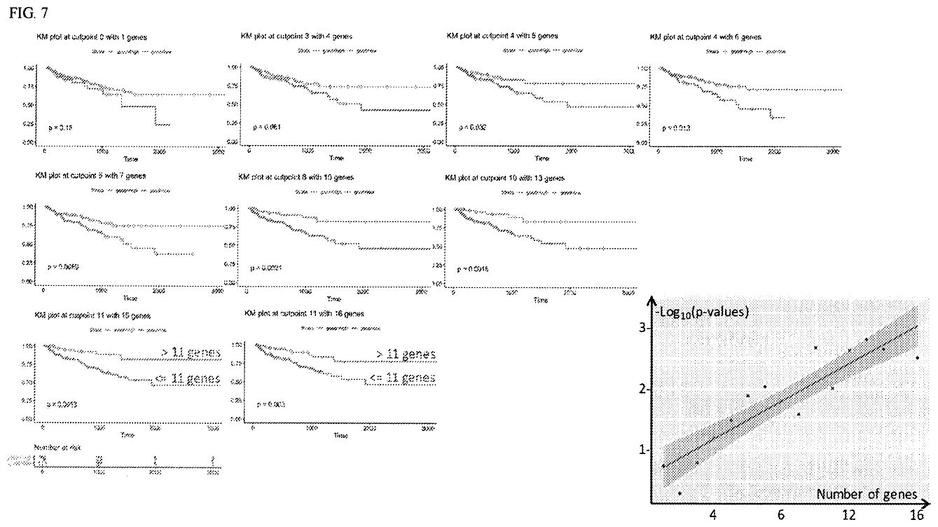

Ranked analysis of the 16 genes which had stable expression within a patient's prostate, varying expression between different patients, and univariately associated with BCR are shown in FIG. 7. Included expression data were from 249 patients with prostate cancer of Gleason grade 4+3 or higher from the cohort of The Cancer Genome Atlas. Each of the 16 genes were scored as contributing to good or bad prognosis in each of the 249 patients. The genes were ranked by how well they contributed to the correct prognosis. The analyses started by including the first-ranked gene, and stepwise including more genes. The Kaplan-Meier plots are examples of including 1, 4, 5, 6, 7, 10, 13, 15, and 16 genes, where the thresholds between two groups are at 0, 3, 4, 4, 5, 8, 10, 11, and 11 genes. The scatter plot shows the −Log₁₀(p-values) as an increasing number of genes are included.

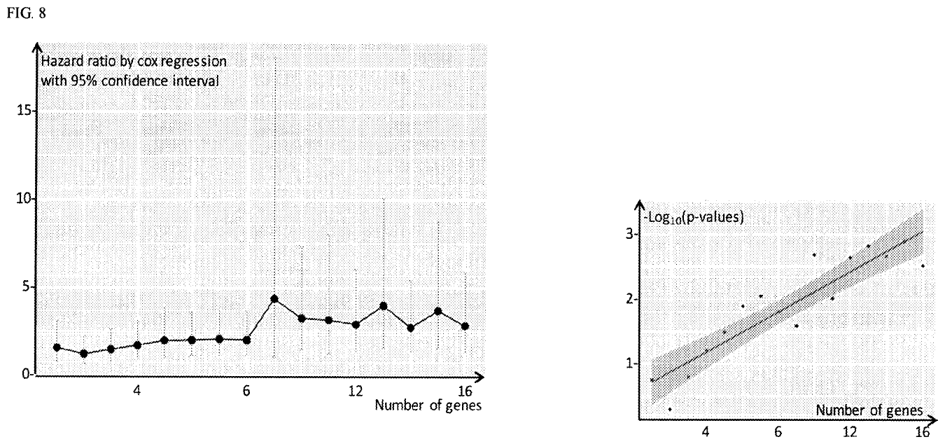

Hazard ration by cox regression with stepwise addition of multiple genes from the 16 genes which had stable expression within a patient's prostate, varying expression between different patients, and univariately associated with BCR are shown in FIG. 8. Included expression data were from 249 patients with prostate cancer of Gleason grade 4+3 or higher from the cohort of The Cancer Genome Atlas. Each of the 16 genes were scored as contributing to good or bad prognosis in each of the 249 patients. The genes were ranked by how well they contributed to the correct prognosis. The analyses started by including the first-ranked gene, and stepwise including more genes. The Kaplan-Meier plots are examples of including 1, 4, 5, 6, 7, 10, 13, 15, and 16 genes, where the thresholds between two groups are at 0, 3, 4, 4, 5, 8, 10, 11, and 11 genes. The scatter plot shows the −Log₁₀(p-values) as an increasing number of genes are included.

The 16 genes were also ranked according to their importance based on ridge penalized regression. An essential parameter of constructing the model is called lambda. The selection of the best lambda is optimized by cross validation, which is dependent on a random seed number. Thus, to find a robust lambda, the analysis was run 100 times with a different seed number each time. The median lambda from these 100 analyses was used further.

The ridge penalized regression provided a ranked list of genes as follows: NOMO3, KLF13, ERAP2, PAM, TMC4, ARHGEF35, DDX58, MICA, ACOT1, C17orf97, IFIH1, CAPN9, CCDC163P, USP18, THNSL2, DDX60 With the resulting model, each of the genes have the following weights:
ACOT1 0.023161455
ARHGEF35 0.031889763
C17orf97 0.018240091
CAPN9 −0.014787891
CCDC163P 0.014548764
DDX58 0.027155063
DDX60 −0.003358558
ERAP2 −0.058139189
IFIH1 0.015176214
KLF13 0.069914438
MICA −0.026302394
NOMO3 0.087060494
PAM 0.047398071
THNSL2 −0.004478799
TMC4 −0.038442598
USP18 0.007242503

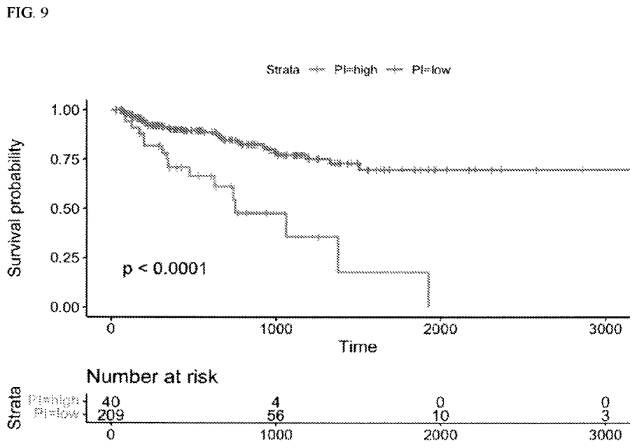

FIG. 9 shows a Kaplan-Meier plot of prostate cancer patients scored as high or low-risk of relapse according to the survival model produced using the ridge penalized regression described above.

TABLE 1
Clinically relevant genes with high inter-patient and low intra-patient heterogeneity.

| | | | | | | HR | p-value | HR | p-value | |
|---|---|---|---|---|---|---|---|---|---|---|
| | | | 25^{th} | Intra-patient | Inter-patient | univar- | univar- | multi- | multi- | |
| Gene | | ICC | percentile | hetero- | hetero- | iate | iate | variate | variate | Accession |
| name | ICC | category | TPM | geneity | geneity | * | * | * | * | number |
| ACOT1 | 0.81 | Good | 8.35 | 0.21 | 0.50 | 0.54 | <0.01 | 0.83 | 0.47 | NM_001037161.2 |
| ARHGEF35 | 0.90 | Good | 6.00 | 0.14 | 0.45 | 1.59 | 0.04 | 1.66 | 0.04 | NM_001003702.3 |
| C17orf97 | 0.89 | Good | 3.08 | 0.19 | 0.60 | 0.58 | 0.04 | 0.92 | 0.77 | NM_001013672.5 |
| CAPN9 | 0.86 | Good | 1.74 | 0.25 | 0.85 | 0.50 | 0.01 | 0.90 | 0.71 | NM_006615.3 |
| CCDC163P | 0.76 | Good | 2.65 | 0.18 | 0.45 | 1.55 | 0.05 | 1.17 | 0.52 | NR_033298.3 |
| DDX58 | 0.92 | Excellent | 34.28 | 0.20 | 0.89 | 1.72 | 0.02 | 1.64 | 0.04 | NM_014314.4 |
| DDX60 | 0.83 | Good | 38.20 | 0.25 | 0.77 | 1.82 | <0.01 | 1.67 | 0.03 | NM_017631.6 |
| ERAP2 | 0.88 | Good | 6.25 | 0.23 | 0.83 | 0.58 | 0.04 | 0.63 | 0.10 | NM_022350.5 |
| IFIH1 | 0.91 | Excellent | 38.26 | 0.16 | 0.57 | 2.05 | <0.01 | 1.83 | 0.01 | NM_022168.4 |
| KLF13 | 0.82 | Good | 1.76 | 0.22 | 0.55 | 2.22 | <0.01 | 1.53 | 0.17 | NM_015995.4 |
| MICA | 0.86 | Good | 6.06 | 0.25 | 0.62 | 0.50 | <0.01 | 0.59 | 0.03 | KY500940.1 |
| NOMO3 | 0.83 | Good | 3.81 | 0.14 | 0.48 | 0.61 | 0.03 | 0.70 | 0.14 | NM_001004067.4 |
| PAM | 0.81 | Good | 64.43 | 0.17 | 0.45 | 1.86 | 0.03 | 1.34 | 0.34 | NM_000919.4 |
| THNSL2 | 0.91 | Excellent | 21.98 | 0.15 | 0.48 | 0.41 | <0.01 | 0.50 | 0.02 | NM_018271.5 |
| TMC4 | 0.79 | Good | 34.67 | 0.17 | 0.45 | 0.60 | 0.02 | 0.74 | 0.20 | NM_001145303.3 |
| USP18 | 0.85 | Good | 15.69 | 0.21 | 0.80 | 1.87 | 0.02 | 1.46 | 0.20 | NM_017414.4 |

ICC: Intraclass correlation coefficient; TPM: Transcripts per million.

### Example 3

This example contains a collection of validation analyses which are platform independent testing of the 16 genes which are associated with biochemical relapse (BCR) after surgery and with heterogeneity agnostic expression levels in prostate cancer. The discovery of the 16 genes was based on data produced by whole-transcriptome RNA-sequencing with the Illumina platform, whereas the validation analyses were performed by real-time reverse-transcription PCR with use of the TaqMan platform.

The association with BCR in the discovery phase was based on a US cohort, published by The Cancer Genome Atlas (47), whereas the validation was performed from an independent Norwegian cohort. The independent validation cohort included RNA samples from malignant and benign prostate tissue and associated clinical data from 247 patients operated 2010 to 2012.

### Materials and Methods

### Patient Cohort and Subcohorts

The studied patients were selected from a prospectively collected cohort of 571 prostate cancer patients treated with radical prostatectomy at Oslo University Hospital-Radiumhospitalet between 2010 and 2012. This total cohort has been described in a set of recent publications (48-50). All patients were treated with prostatectomy with curative intent. From each patient, fresh-frozen tissue samples were available from between three to eight sites.

A subcohort of 247 patients was included in the current analyses. To enable objective follow-up data, post-surgical PSA values have been collected through correspondence with general practitioners, review of hospital records, and from Fürst Medical Laboratories (Oslo, Norway). For all 247 patients, RNA was available from at least one malignant tissue, and in total we included 584 tissue samples, of which 438 are malignant tissue. 103 of the 247 patients (42%) have experienced biochemical relapse (BCR), and the median follow-up time for the remaining 144 patients was 9.9 years. Patients with Gleason grade group >=3 (Gleason score 4+3 or above) were selected for testing within an a priori subcohort with elevated risk of BCR. Within this subcohort of 156 patients, 89 (57.1%) have had BCR, and median follow-up time for the remaining 67 patients was 10.1 years.

Histopathological re-evaluation of the surgically removed specimens was performed according to the 2014 International Society of Urological Pathology (ISUP) Modified Gleason system (51). For analyses using fresh-frozen tissue, the area surrounding where the tissue sample was collected was used to evaluate Gleason score. For all radical prostatectomy specimens, multifocality was assessed and it was determined from which focus each tissue sample was collected. Tumours were defined as different foci when clearly separated by at least 2 to 4 mm and showing different tissue morphology.

RNA Isolation, cDNA Synthesis, RT-PCR, TaqMan RT-PCR Assays

RNA was isolated from fresh-frozen tissue samples with the AllPrep DNA/RNA/miRNA Universal kit (Qiagen, Venlo, Netherlands). Complementary DNA (cDNA) was generated by reverse transcription of total RNA using the High Capacity cDNA Reverse Transcription kit (Thermo Fisher Scientific, Waltham, MA, USA) according to the manufacturer's protocols. Semi-quantitative RNA expression levels of 16 genes of interest and three reference genes were determined with real-time reverse transcription polymerase chain reaction (RT-PCR), in a reaction volume of 10 μl, using TaqMan Universal Master Mix II, with UNG (Thermo Fisher Scientific) and TaqMan Gene Expression assays (Thermo Fisher Scientific) as listed in Table 2. For two of the genes, custom designed assays were developed using the Primer Express software, targeting exon-exon junctions of commonly expressed transcript variants. The custom designed probes were ordered from Thermo Fisher Scientific with the same modifications as the probes in the pre-designed assays (FAM on the 5′-end, and a non-fluorescent quencher [NFQ] and a minor groove binder [MGB] on the 3′-end). The oligo forward and reverse primers were ordered from Eurogentec (Searing, Belgium).

Three endogenous control genes were included. SLC4A1AP was identified as a particularly stably expressed gene within RNA-sequencing data from 87 benign and malignant prostate tissue samples (52). ABL1 and G6PD were selected as reference genes based on their already established roles and performance as prostate cancer reference genes (53, 54), as well as being sufficiently stably expressed in the RNA-seq data from which SLC4A1AP was identified (52).

TABLE 2
TaqMan assays for the 16 biomarker and 3 reference
genes. Assays, both custom and pre-designed,
contain a TaqMan minor groove binder (MGB)
probe modified with a reporter dye (FAM, 5′-end)
and a non-fluorescent quencher (NFQ, 3′-end).
The custom designed assays targeting MICA and
NOMO3 were both targeting exon-exon junctions.
For MICA, the assay targeted the exons 4 and 5
of the transcript ENST00000449934 (forward primer
ATGGAACACAGCGGGAATCA (SEQ ID NO: 1), reverse
primer CAGCAACAGCAGAAACATGGA (SEQ ID NO: 2),
and probe TGCCCTCTGGGAAAG (SEQ ID NO: 3)) and
for NOMO3 the assay targeted the exons 29 and
30 of the transcript ENST00000263012 (forward
primer CCGCAGTGGGCTACCATAAA (SEQ ID NO: 4),
reverse primer AATGGCAGGGCAATGTAGGA (SEQ ID NO:
5), and probe TCCCACGAGGAAGC (SEQ ID NO: 6)).

| Gene | | Real-time |
|---|---|---|
| symbol | Ensembl gene ID | RT-PCR assay |
| Biomarker | | |
| genes | | |
| ACOT1 | ENSG00000184227 | Hs02598489_mH |
| ARHGEF35 | ENSG00000213214 | Hs04194829_gH |
| C17orf97 | ENSG00000187624 | Hs03004183_m1 |
| CAPN9 | ENSG00000135773 | Hs01005004_m1 |
| CCDC163P | ENSG00000280670 | Hs01373214_g1 |
| DDX58 | ENSG00000107201 | Hs01061436_m1 |
| DDX60 | ENSG00000137628 | Hs01102732_m1 |
| ERAP2 | ENSG00000164308 | Hs01073631_m1 |
| IFIH1 | ENSG00000115267 | Hs01070322_m1 |
| KLF13 | ENSG00000169926 | Hs00429818_m1 |
| MICA | ENSG00000204520 | Custom designed |
| NOMO3 | ENSG00000103226 | Custom designed |
| PAM | ENSG00000145730 | Hs01084035_m1 |
| THNSL2 | ENSG00000144115 | Hs01016388_g1 |
| TMC4 | ENSG00000167608 | Hs01547178_g1 |
| USP18 | ENSG00000184979 | Hs00276441_m1 |
| Reference | | |
| genes | | |
| ABL1 | ENSG00000097007 | Hs01104728_m1 |
| G6PD | ENSG00000160211 | Hs00166169_m1 |
| SLC4A1AP | ENSG00000163798 | Hs00250835_m1 |

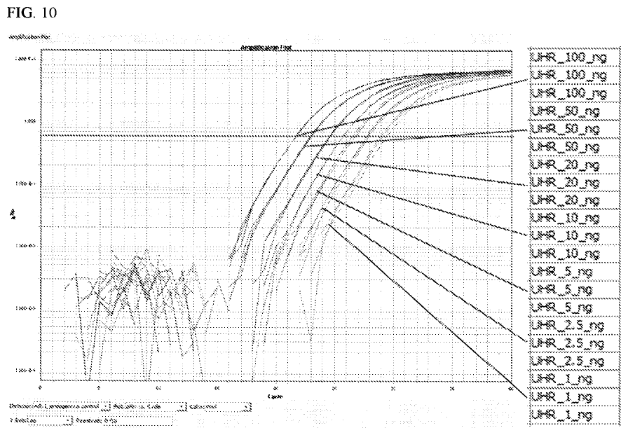

All samples were run on an ABI 7900HT Fast Real-time PCR System (Thermo Fisher Scientific), with 10 ng cDNA input in each reaction. 118 of the samples were run in triplicate and the remaining 510 were run in single reactions. Cycle threshold (Ct) values were obtained for each assay for all samples, and a serial dilution of Universal Human Reference RNA (FIG. 10).

### Processing of Real-Time RT-PCR Data

Quantification for each of the 16 genes and 3 reference genes was based on their respective Ct values in each sample, and on standard curves. The standard curves were calculated based on a serial dilution of the Human Reference RNA (UHR; 100, 50, 20, 10, 5, 2.5, and 1 ng cDNA inputs), and used to calculate the assay-specific PCR effectivity. A Q (quantity) value was produced for each of the 16 genes of interest in each sample. This takes the PCR effectivity into account, and the ratio between the relative quantities of genes of interest and the average of the three control genes. Samples with Ct >35 were considered as having no expression, and thus assigned with a Q equaling the lowest value among the samples with valid measurements. For samples run in triplicate, the median Q was used further.

Association with Biochemical Relapse

### Thresholds for High and Low Expression

For each gene of interest, the R-package survminer, version 0.4.9 (55) was used to determine the optimal threshold, and for assigning samples to a high and low expressing group. The threshold was set at the optimal value for univariate association with BCR.

### Kaplan-Meier Plots

Kaplan-Meier plots were generated with the R-package survminer, version 0.4.9 (55) to visualize how each gene's high and low expressing groups of patients may have different BCR-free proportions along the x-axis of time to BCR. Log-rank tests were calculated to produce statistical significance values for the difference between the high and low expressing groups for each gene, related to the patients' time to BCR.

### Hazard Ratios

Hazard ratios were calculated with the R-package survival, version 3.2-13 (56). Univariable models were generated for each gene based on time to BCR for patients dichotomized by high or low expression levels (as described above).

### Multi-Gene Signature

Multi-gene signatures were generated to identify patients with high risk of BCR. Categorized gene expression levels were described as favourable for patient prognosis if univariable Cox regression yielded a hazard ratio of 1 or less. Patients were then assigned as having favourable or unfavourable expression levels for each gene, and the number of genes with favourable expression were used to dichotomize samples into high and low-risk groups using different thresholds.

### Intrapatient Heterogeneity Analyses

For all patients where there were at least two malignant tissue samples per patient, and the cDNA met the quality criterion, a comparison of the high vs. low expression category per gene was made. A corresponding number for random pairs of samples within the sample was calculated.

Principal components analysis (PCA) was performed with input of data from all malignant tissue samples meeting the cDNA quality control criterion.

### Results

Three successively filtered input datasets were analysed:
1. All patients in validation cohort with one malignant sample per patient (n=247);
2. Patients with quality controlled cDNA input (n=233);
3. Patients with intermediate and high-risk cancers, i.e. Gleason grade group >=3; and quality controlled cDNA input (n=147).
   1. All Patients in Validation Cohort with One Malignant Sample Per Patient

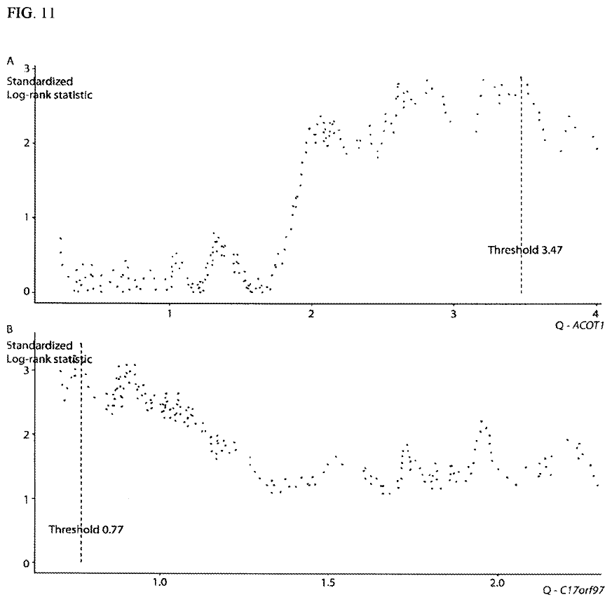

The 247 patients for which at least one RNA sample from malignant tissue was included in the following analysis. For each of the 16 genes, thresholds for scoring the malignant samples as having either «high» or «low» expression was set to maximize the genes' association with BCR. ACOT1 is an example of a gene which would produce significant association with BCR at a wide range of thresholds, whereas C17orf97 had significant association with BCR at a more narrow range of threshold (FIG. 11).

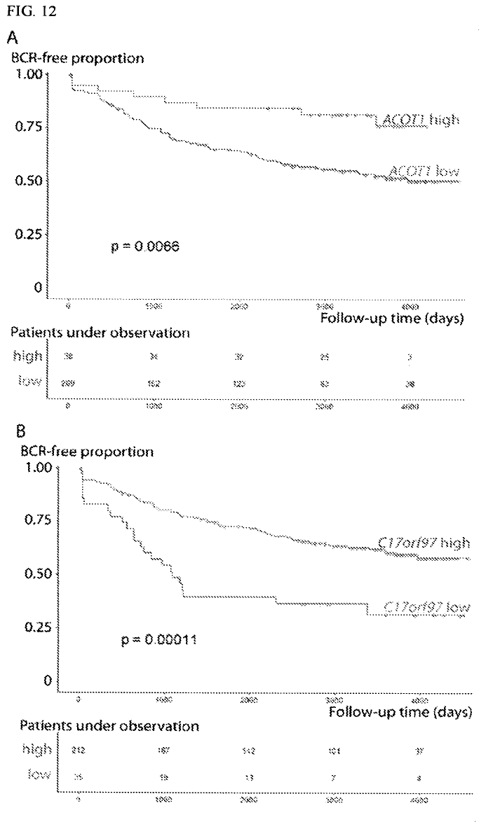

Kaplan-Meier plots were produced for all 16 genes and shown for C17orf97 and ACOT1 in FIG. 12.

Out of the 16 genes, 9 were statistically significantly (p<0.05) associated with BCR in univariate analyses (Table 3).

TABLE 3
Genes and associations with BCR among the
247 patients. The numbers of patients in
the poor and good groups are indicated.

| Gene | P-value | Poor | Good |
|---|---|---|---|
| C17orf97 | 0.00011 | 35 | 212 |
| MICA | 0.0035 | 48 | 199 |
| ACOT1 | 0.0066 | 209 | 38 |
| DDX58 | 0.0084 | 172 | 75 |
| PAM | 0.0088 | 37 | 210 |
| THNSL2 | 0.0090 | 211 | 36 |
| KLF13 | 0.013 | 95 | 152 |
| ERAP2 | 0.029 | 28 | 219 |
| USP18 | 0.040 | 85 | 162 |
| TMC4 | 0.063 | 24 | 223 |
| IFIH1 | 0.077 | 28 | 219 |
| CCDC163P | 0.080 | 77 | 170 |
| NOMO3 | 0.094 | 46 | 201 |
| DDX60 | 0.10 | 25 | 222 |
| CAPN9 | 0.12 | 123 | 124 |
| ARHGEF35 | 0.21 | 175 | 72 |

2. Patients with High Quality cDNA Input

For some of the genes with generally weak expression, there was a tendency that when they had undetermined Ct-value, this could be due to poor cDNA quality just as well as low expression of the gene. ACOT1 is an example of a gene for which had undetermined expression level for many samples for which the expression of control genes were low.

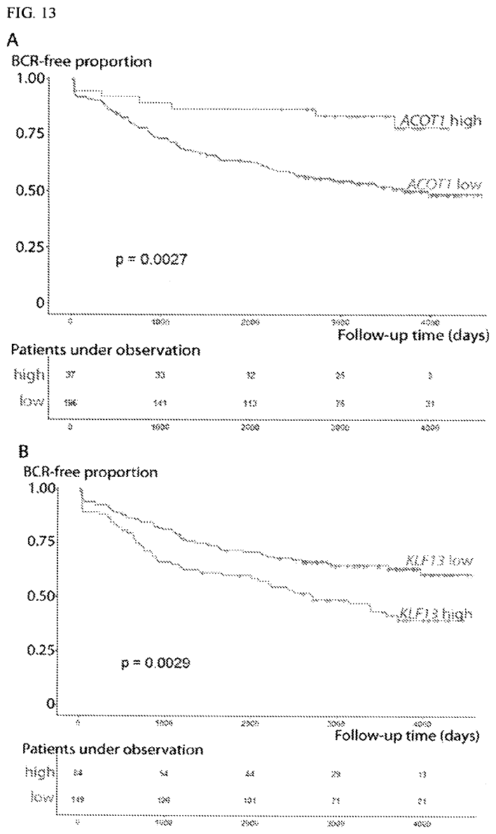

Analyses were performed where only the 233 patients from whom RNA from malignant tissue samples had average expression of the three control genes within two standard deviations of the average of the 247 samples. The actual cDNA input threshold was then that the three control genes should have an average-Ct below 31.739 for a sample to be considered further. Kaplan-Meier plots were produced for all 16 genes, where those for ACOT1 and KLF13 are shown in FIG. 13.

Of note, C17orf97 had p<0.0001, and out of the 16 genes, 10 were statistically significantly (p<0.05) associated with BCR in univariate analyses (Table 4).

TABLE 4
Genes and associations with BCR among the 233 patients for
whom the cDNA input passed quality control. Only samples
with average expression of the three control genes within 2
standard deviations from the average were included.

| Gene | P-value | Poor | Good |
|---|---|---|---|
| C17orf97 | <0.0001 | 32 | 201 |
| ACOT1 | 0.0027 | 196 | 37 |
| KLF13 | 0.0029 | 84 | 149 |
| DDX58 | 0.0039 | 160 | 73 |
| MICA | 0.0061 | 46 | 187 |
| THNSL2 | 0.0078 | 183 | 50 |
| PAM | 0.0092 | 46 | 187 |
| CCDC163P | 0.034 | 69 | 164 |
| ERAP2 | 0.038 | 28 | 205 |
| TMC4 | 0.042 | 23 | 210 |
| USP18 | 0.053 | 53 | 180 |
| IFIH1 | 0.088 | 28 | 205 |
| NOMO3 | 0.091 | 39 | 194 |
| CAPN9 | 0.14 | 41 | 192 |
| DDX60 | 0.16 | 44 | 189 |
| ARHGEF35 | 0.18 | 162 | 71 |

### Hazard Ratios

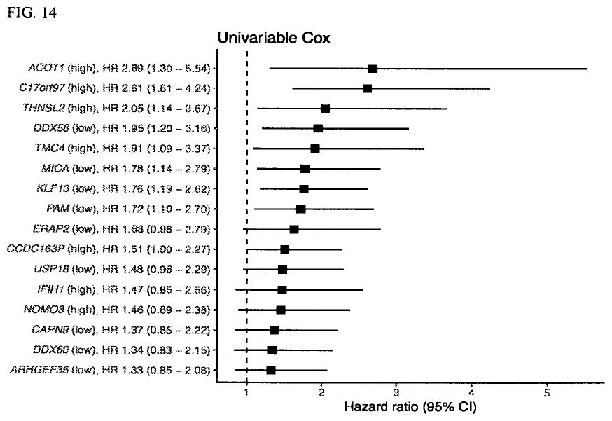

The hazard ratio was calculated for all 16 genes (FIG. 14) for the 233 patients.

### Multi-Gene Signature

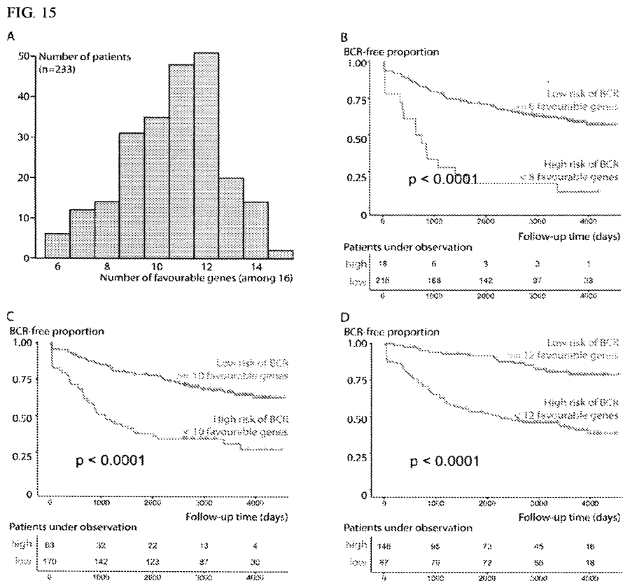

For each of the 233 patients, it was counted how many of the 16 genes that had expression level in the favourable category. The patients had between 6 and 15 genes in the favourable category, and a distribution plot is shown in FIG. 15A. Kaplan-Meier plots with thresholds at three selected thresholds are shown in FIG. 15B-D.

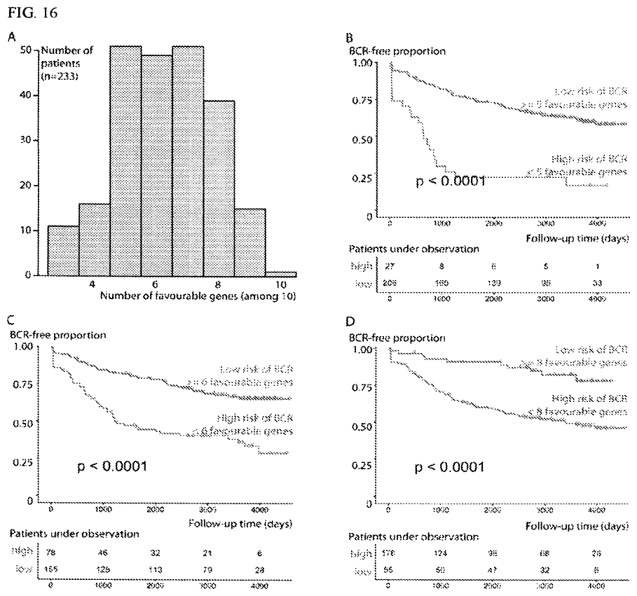

We also restricted these analyses to the ten genes with univariate statistically significant association to BCR (p<0.05; Table 4). Here, for each of the 233 patients, it was counted how many of the 10 genes that had expression level in the favourable category. The patients had between 5 and 10 genes in the favourable category, and a distribution plot is shown in FIG. 16A. Kaplan-Meier plots with thresholds at three selected thresholds are shown in FIG. 16B-D.

3. Patients with Intermediate and High-Risk Cancers

One relevant use of genes with expression levels that are associated with BCR is to select patients with already known high or intermediate risk of BCR into additional risk groups. Patients with additionally high risk based on the gene expression can for example be selected for adjuvant therapy following a radical prostatectomy.

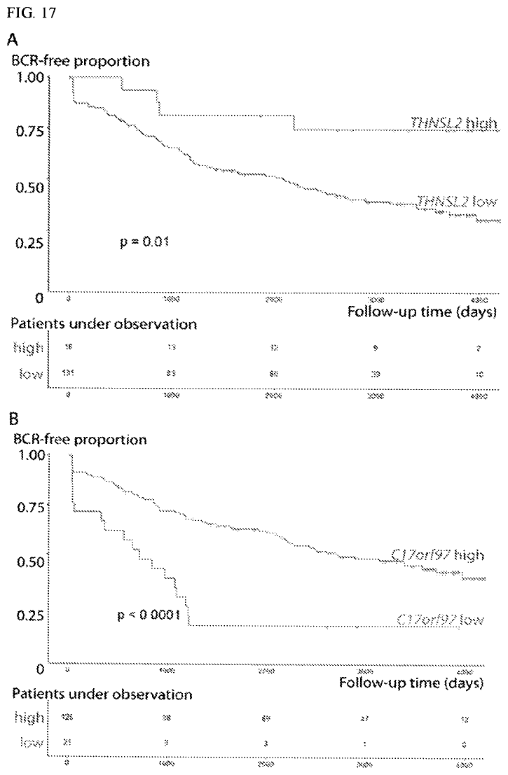

Analyses were performed where the 147 patients with Gleason grade group of at least 3 (Gleason score 7b; pattern 4+3) and with cDNA passing quality control. Kaplan-Meier plots were produced for all 16 genes and those of THNSL2 and C17orf97 are shown in FIG. 17.

Of note, C17orf97 had p<0.0001, and out of the 16 genes, 8 were statistically significantly (p<0.05) associated with BCR in univariate analyses (Table 5).

TABLE 5
Genes and associations with BCR among the 147
patients with Gleason grade group of
at least 3. Further, only patients from whom the
cDNA input passed quality controlled were
included (average expression of the three control genes
within 2 standard deviations from the average).

| Gene | P-value | Poor | Good |
|---|---|---|---|
| C17orf97 | <0.0001 | 21 | 126 |
| ACOT1 | 0.01 | 131 | 16 |
| THNSL2 | 0.01 | 131 | 16 |
| DDX58 | 0.012 | 101 | 46 |
| ERAP2 | 0.023 | 20 | 127 |
| MICA | 0.024 | 46 | 101 |
| USP18 | 0.025 | 31 | 116 |
| KLF13 | 0.041 | 90 | 57 |
| CAPN9 | 0.053 | 17 | 130 |
| ARHGEF35 | 0.055 | 103 | 44 |
| PAM | 0.058 | 33 | 114 |
| DDX60 | 0.058 | 15 | 132 |
| TMC4 | 0.069 | 44 | 103 |
| NOMO3 | 0.083 | 100 | 47 |
| CCDC163P | 0.1 | 96 | 51 |
| IFIH1 | 0.11 | 18 | 129 |

### Intrapatient Heterogeneity Across Multiple Tissues Per Patient

Additional samples per patient was analysed for 12 of the 16 genes. The genes were selected by their statistical significance value in univariate association with BCR among as in Table 4 (233 patients with high quality cDNA input). The total number of samples analysed was 584, of which 438 were from malignant tissue, and 412 which met the cDNA quality control criterion.

For the 113 patients where there were at least two malignant tissue samples per patient, and the cDNA met the quality criterion, there were 303 malignant sample pairs, and thus 3636 comparisons (12 genes×303 sample pairs). A comparison of the high vs. low expression category per gene, reveal that 83.3 percent score within the same category. The corresponding percentage for any random pair of samples within the sample set is 68.0.

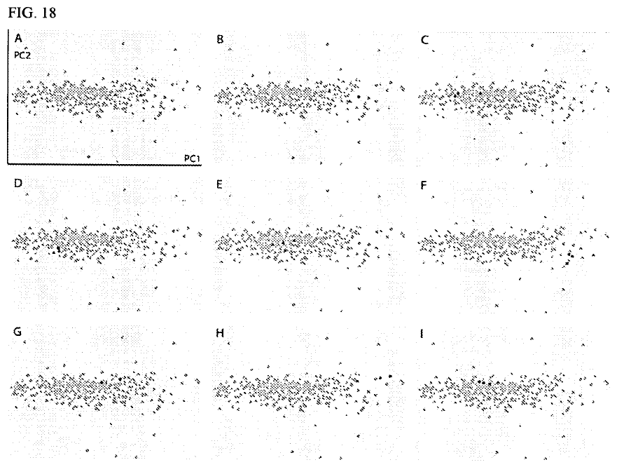

Principal components analysis (PCA) was generated for 412 malignant tissue samples with input Q values for the 12 genes which were analysed for multiple samples per patient (FIG. 18). Plots for each of the 113 patients from whom multiple malignant tissue samples were analysed, and plots for a selection of 9 patients are provided in FIG. 18. There was a clear tendency that samples from the same patient cluster together.

Three Example Patients, the Young, the Miracle, and the Unlucky, and their Benefit from the ProClass Test

Three men, here referred to as the young, the miracle, and the unlucky, are here described with brief clinical stories and ProClass results.

The young man was at 47 years of age diagnosed with prostate cancer. He has noted a clustering of cancer cases in his family, but no germline genetic testing has been performed. His prostate cancer was classified with Gleason patterns 3+5 prostate cancer (Gleason score 8 and Gleason grade group 4 (GG4)). In the cohort, 16 of 30 patients with GG4 have experienced BCR. The young man had free surgical margins and had a presurgical PSA serum concentration of 14 ng per ml.

The patient has since surgery taken a blood test measuring the PSA concentration 45 times. A test taken after 3.2 years was the first one above the detection threshold 0.2 ng/ml, and thereby indicated BCR. Subsequent magnetic resonance imaging (MRI) did not conclude on actual metastases, but he started hormone deprivation and had salvage radiation treatment directed to prostate and pelvic regions. His PSA has thereafter dropped, and stayed low for four years, until a second BCR. Two years later, he has still no certain overt metastases visible by MR or PET-PSMA.

Altogether three tissue samples from his radical prostatectomy specimen have been analysed by the ProClass test. One of these was from a normal appearing tissue and two from a malignant tumour. The two malignant tissue samples were in good agreement across the geneset (FIG. 18A). For example, all the three tissues had low levels of C17orf97. This is associated with increased risk of BCR (FIG. 12, FIG. 17). For the ten gene signature, the young man's prostate tissue has five genes with expression value in the favourable category, which is below the threshold indicating that he is within the third of the patients with the highest risk of relapsing cancer (FIG. 7C).

The ProClass test results are in favour of considering adjuvant treatment after the surgery (e.g., chemotherapy and/or hormonal treatment) for the young man.

Two other men, the miracle man and the unlucky man, both have prostate cancer with GG5. Patients with GG5 cancer have a quite poor prognosis, where more than half experience BCR before 5 years after surgery. Further, the prostatectomy specimen from both men had unfree surgical margins, and thus the two men had even higher risk of relapse.

The miracle man has almost 11 years after the prostatectomy tested his PSA 22 times, and every time with a negative result.

Four tissue samples from the prostatectomy specimen were fresh frozen, and RNA has been isolated and tested. The three malignant tissue samples were from two different malignant tumour foci, and yet all three were in good agreement across the geneset (FIG. 18B). For example, in all four tissue samples, the expression of the THNSL2 gene was above the threshold. The same was the case for the C17orf97 gene. For both these genes, this is associated with a favourable prognosis (FIG. 17). For the ten gene signature, the miracle man's prostate tissue has 8 genes with expression value in the favourable category, which indicates that he is among the 24% of the patients with the lowest risk of BCR (FIG. 16D). Thus, the miracle man has a ProClass test result indicating a low risk of relapsing cancer.

The ProClass test results do not give additional support of considering adjuvant treatment after the surgery (e.g. chemotherapy and/or hormonal treatment) for the miracle man.

The unlucky man has tested his serum concentration of PSA 30 times, and after 1.8 years it changed from negative to positive, and thus BCR. A subsequent MRI showed a residual tumour, and he started on hormonal treatment and received salvage radiation. His PSA dropped to zero. However, two years later, he got metastases in the lungs, liver and bones. Despite several types of treatments he got gradually worse, and five years after initial surgery he died due to his cancer.

Four tissue samples from the prostatectomy specimen were fresh frozen, and RNA has been isolated and tested. The three malignant tissue samples were from the same malignant tumour focus, and were in good agreement across the geneset (FIG. 18C). For example, in all four tissue samples, the expression of the THNSL2 gene was below the threshold. The same was the case for the C17orf97 gene. For both these genes, this is associated with a poor prognosis (FIG. 17). For the ten gene signature, the unlucky man's prostate tissue has only two genes with expression value in the favourable category, which is far below the threshold of 4 genes for identifying the twelve percent with the highest risk of relapsing cancer (FIG. 7B). Thus, the unlucky man has a ProClass test result with clear indication of a high risk of relapsing cancer.

The ProClass test results are in favour of considering adjuvant treatment after the surgery (e.g. chemotherapy and/or hormonal treatment) for the unlucky man.

### REFERENCES

1. Sung H, Ferlay J, Siegel R L, Laversanne M, Soerjomataram I, Jemal A, et al. Global cancer statistics 2020: GLOBOCAN estimates of incidence and mortality worldwide for 36 cancers in 185 countries. CA: a cancer journal for clinicians. 2021; 71 (3): 209-49.
2. Ruijter E T, Van de Kaa C A, Schalken J A, Debruyne F M, Ruiter D J. Histological grade heterogeneity in multifocal prostate cancer. Biological and clinical implications. The Journal of pathology. 1996; 180 (3): 295-9.
3. Løvf M, Zhao S, Axcrona U, Johannessen B, Bakken A C, Carm K T, et al. Multifocal primary prostate cancer exhibits high degree of genomic heterogeneity. European urology. 2019; 75 (3): 498-505.
4. Cancer Genome Atlas Research Network. The molecular taxonomy of primary prostate cancer. Cell. 2015; 163 (4): 1011-25.
5. Kumar-Sinha C, Tomlins S A, Chinnaiyan A M. Recurrent gene fusions in prostate cancer. Nature Reviews Cancer. 2008; 8 (7): 497-511.
6. Tomlins S A, Laxman B, Varambally S, Cao X, Yu J, Helgeson B E, et al. Role of the TMPRSS2-ERG gene fusion in prostate cancer. Neoplasia. 2008; 10 (2): 177-IN9.
7. Tomlins S A, Mehra R, Rhodes D R, Smith L R, Roulston D, Helgeson B E, et al. TMPRSS2: ETV4 gene fusions define a third molecular subtype of prostate cancer. Cancer research. 2006; 66 (7): 3396-400.
8. Tomlins S A, Bjartell A, Chinnaiyan A M, Jenster G, Nam R K, Rubin M A, et al. ETS gene fusions in prostate cancer: from discovery to daily clinical practice. European urology. 2009; 56 (2): 275-86.
9. Pettersson A, Graff R E, Bauer S R, Pitt M J, Lis R T, Stack E C, et al. The TMPRSS2: ERG rearrangement, ERG expression, and prostate cancer outcomes: a cohort study and meta-analysis. Cancer Epidemiology and Prevention Biomarkers. 2012; 21 (9): 1497-509.
10. Paulo P, Barros-Silva J D, Ribeiro F R, Ramalho-Carvalho J, Jerónimo C, Henrique R, et al. FLI1 is a novel ETS transcription factor involved in gene fusions in prostate cancer. Genes, Chromosomes and Cancer. 2012; 51 (3): 240-9.
11. Pflueger D, Terry S, Sboner A, Habegger L, Esgueva R, Lin P-C, et al. Discovery of non-ETS gene fusions in human prostate cancer using next-generation RNA sequencing. Genome research. 2011; 21 (1): 56-67.
12. Barbieri C E, Baca S C, Lawrence M S, Demichelis F, Blattner M, Theurillat J-P, et al. Exome sequencing identifies recurrent SPOP, FOXA1 and MED12 mutations in prostate cancer. Nature genetics. 2012; 44 (6): 685-9.
13. Salami S S, Hovelson D H, Kaplan J B, Mathieu R, Udager A M, Curci N E, et al. Transcriptomic heterogeneity in multifocal prostate cancer. JCI insight. 2018; 3 (21).
14. Epstein J I, Egevad L, Amin M B, Delahunt B, Srigley J R, Humphrey P A. The 2014 International Society of Urological Pathology (ISUP) consensus conference on Gleason grading of prostatic carcinoma. The American journal of surgical pathology. 2016; 40 (2): 244-52.
15. Bolger A M, Lohse M, Usadel B. Trimmomatic: a flexible trimmer for Illumina sequence data. Bioinformatics. 2014; 30 (15): 2114-20.
16. Andrews S. FastQC: A Quality Control Tool for High Throughput Sequence Data. 2010.
17. Ewels P, Magnusson M, Lundin S, Käller M. MultiQC: summarize analysis results for multiple tools and samples in a single report. Bioinformatics. 2016; 32 (19): 3047-8.
18. Anders S, Pyl P T, Huber W. HTSeq—a Python framework to work with high-throughput sequencing data. Bioinformatics. 2015; 31 (2): 166-9.
19. Ritchie M E, Phipson B, Wu D, Hu Y, Law C W, Shi W, et al. limma powers differential expression analyses for RNA-sequencing and microarray studies. Nucleic acids research. 2015; 43 (7): e47-e.
20. McPherson A, Hormozdiari F, Zayed A, Giuliany R, Ha G, Sun M G, et al. deFuse: an algorithm for gene fusion discovery in tumor RNA-Seq data. PLOS Comput Biol. 2011; 7 (5): e1001138.
21. Nicorici D, Şatalan M, Edgren H, Kangaspeska S, Murumägi A, Kallioniemi O, et al. FusionCatcher—a tool for finding somatic fusion genes in paired-end RNA-sequencing data. BioRxiv. 2014:011650.
22. Haas B J, Dobin A, Stransky N, Li B, Yang X, Tickle T, et al. STAR-Fusion: fast and accurate fusion transcript detection from RNA-Seq. BioRxiv. 2017:120295.
23. Pederzoli F, Bandini M, Marandino L, Ali S M, Madison R, Chung J, et al. Targetable gene fusions and aberrations in genitourinary oncology. Nature Reviews Urology. 2020; 17 (11): 613-25.
24. McKenna A, Hanna M, Banks E, Sivachenko A, Cibulskis K, Kernytsky A, et al. The Genome Analysis Toolkit: a MapReduce framework for analyzing next-generation DNA sequencing data. Genome research. 2010; 20 (9): 1297-303.
25. Li H, Handsaker B, Wysoker A, Fennell T, Ruan J, Homer N, et al. The sequence alignment/map format and SAMtools. Bioinformatics. 2009; 25 (16): 2078-9.
26. Bates D, Mächler M, Bolker B, Walker S. Fitting linear mixed-effects models using lme4. ar Xiv preprint arXiv: 14065823. 2014.
27. Lüdecke D, Makowski D, Waggoner P. Performance: assessment of regression models performance. R package version 04. 2019; 2.
28. Kremer A, Kremer T, Kristiansen G, Tolkach Y. Where is the limit of prostate cancer biomarker research? Systematic investigation of potential prognostic and diagnostic biomarkers. BMC urology. 2019; 19 (1): 1-10.
29. Love M I, Huber W, Anders S. Moderated estimation of fold change and dispersion for RNA-seq data with DESeq2. Genome biology. 2014; 15 (12): 550.
30. Zhu A, Ibrahim J G, Love M I. Heavy-tailed prior distributions for sequence count data:

removing the noise and preserving large differences. Bioinformatics. 2019; 35 (12): 2084-92.
31. Ignatiadis N, Klaus B, Zaugg J B, Huber W. Data-driven hypothesis weighting increases detection power in genome-scale multiple testing. Nature methods. 2016; 13 (7): 577-80.
32. Subramanian A, Tamayo P, Mootha V K, Mukherjee S, Ebert B L, Gillette M A, et al. Gene set enrichment analysis: a knowledge-based approach for interpreting genome-wide expression profiles. Proceedings of the National Academy of Sciences. 2005; 102 (43): 15545-50.
33. Luo W, Friedman M S, Shedden K, Hankenson K D, Woolf P J. GAGE: generally applicable gene set enrichment for pathway analysis. BMC bioinformatics. 2009; 10 (1): 161.
34. Sergushichev A. An algorithm for fast preranked gene set enrichment analysis using cumulative statistic calculation. BioRxiv. 2016:060012.
35. Liberzon A, Subramanian A, Pinchback R, Thorvaldsdóttir H, Tamayo P, Mesirov J P. Molecular signatures database (MSigDB) 3.0. Bioinformatics. 2011; 27 (12): 1739-40.
36. Benaglia T, Chauveau D, Hunter D, Young D. mixtools: An R Package for Analyzing Finite Mixture Models. Journal of Statistical Software. 2009; 32 (6): 1-29.
37. Carm K T, Hoff A M, Bakken A C, Axcrona U, Axcrona K, Lothe R A, et al. Interfocal heterogeneity challenges the clinical usefulness of molecular classification of primary prostate cancer. Scientific reports. 2019; 9 (1): 1-6.
38. Boormans J L, Korsten H, Ziel-van der Made A J, van Leenders G J, de Vos C V, Jenster G, et al. Identification of TDRDI as a direct target gene of ERG in primary prostate cancer. International journal of cancer. 2013; 133 (2): 335-45.
39. Miyashita M, Oshiumi H, Matsumoto M, Seya T. DDX60, a DEXD/H box helicase, is a novel antiviral factor promoting RIG-I-like receptor-mediated signaling. Molecular and cellular biology. 2011; 31 (18): 3802-19.
40. Wu Y, Wu X, Wu L, Wang X, Liu Z. The anticancer functions of RIG-I-like receptors, RIG-I and MDA5, and their applications in cancer therapy. Translational Research. 2017; 190:51-60.
41. de Andrade L F, Tay R E, Pan D, Luoma A M, Ito Y, Badrinath S, et al. Antibody-mediated inhibition of MICA and MICB shedding promotes NK cell-driven tumor immunity. Science. 2018; 359 (6383): 1537-42.
42. Cifaldi L, Monaco E L, Forloni M, Giorda E, Lorenzi S, Petrini S, et al. Natural killer cells efficiently reject lymphoma silenced for the endoplasmic reticulum aminopeptidase associated with antigen processing. Cancer research. 2011; 71 (5): 1597-606.
43. Trendel J A, Ellis N, Sarver J G, Klis W A, Dhananjeyan M, Bykowski C A, et al. Catalytically active peptidylglycine α-amidating monooxygenase in the media of androgen-independent prostate cancer cell lines. Journal of biomolecular screening. 2008; 13 (8): 804-9.
44. Tan Y, Zhou G, Wang X, Chen W, Gao H. USP18 promotes breast cancer growth by upregulating EGFR and activating the AKT/Skp2 pathway. International journal of oncology. 2018; 53 (1): 371-83.
45. Peng P, Wu W, Zhao J, Song S, Wang X, Jia D, et al. Decreased expression of Calpain-9 predicts unfavorable prognosis in patients with gastric cancer. Scientific reports. 2016; 6 (1): 1-11.
46. Wang Q, Peng R, Wang B, Wang J, Yu W, Liu Y, et al. Transcription factor KLF13 inhibits AKT activation and suppresses the growth of prostate carcinoma cells. Cancer Biomarkers. 2018; 22 (3): 533-41.
47. The Cancer Genome Atlas Research Network. The molecular taxonomy of primary prostate cancer. Cell. 2015; 163 (4): 1011-25.
48. Kidd S G, Bogaard M, Carm K T, Bakken A C, Maltau A M V, Løvf M, et al. In situ expression of ERG protein in the context of tumor heterogeneity identifies prostate cancer patients with inferior prognosis. Molecular Oncology. 2022; 16 (15): 2810-22.
49. Kidd S G, Carm K T, Bogaard M, Olsen L G, Bakken A C, Løvf M, et al. High expression of SCHLAP1 in primary prostate cancer is an independent predictor of biochemical recurrence, despite substantial heterogeneity. Neoplasia. 2021; 23 (6): 634-41.
50. Løvf M, Zhao S, Axcrona U, Johannessen B, Bakken A C, Carm K T, et al. Multifocal primary prostate cancer exhibits high degree of genomic heterogeneity. Eur Urol. 2019; 75 (3): 498-505.
51. Epstein J I, Egevad L, Amin M B, Delahunt B, Srigley J R, Humphrey P A. The 2014 International Society of Urological Pathology (ISUP) consensus conference on Gleason grading of prostatic carcinoma: Definition of grading patterns and proposal for a new grading system. Am J Surg Pathol. 2016; 40 (2): 244-52.
52. Strømme J M, Johannessen B, Kidd S G, Bogaard M, Carm K T, Zhang X, et al. Expressed prognostic biomarkers for primary prostate cancer independent of multifocality and transcriptome heterogeneity. Cancer gene therapy. 2022; 29 (8-9): 1276-84.
53. Nna E, Tothill I E, Ludeman L, Bailey T. Endogenous control genes in prostate cells: evaluation of gene expression using ‘real-time’ quantitative polymerase chain reaction. Medical principles and practice: international journal of the Kuwait University, Health Science Centre. 2010; 19 (6): 433-9.
54. Carm K T, Hoff A M, Bakken A C, Axcrona U, Axcrona K, Lothe R A, et al. Interfocal heterogeneity challenges the clinical usefulness of molecular classification of primary prostate cancer. Sci Rep. 2019; 9 (1): 13579.
55. Kassambara A, Kosinski M, Biecek P, Fabian S. survminer: Drawing survival curves using ‘ggplot2’. R package version 0.4.9 https://CRAN.R-project.org/package=survminer. 2021.
56. Therneau T M, Lumley T, Atkinson E, Crowson C. survival: A package for survival analysis in R. R package version 3.2-13 https://CRAN.R-project.org/package=survival. 2021.

All publications, patents, patent applications and accession numbers mentioned in the above specification are herein incorporated by reference in their entirety. Although the invention has been described in connection with specific embodiments, it should be understood that the invention as claimed should not be unduly limited to such specific embodiments. Indeed, various modifications and variations of the described compositions and methods of the invention will be apparent to those of ordinary skill in the art and are intended to be within the scope of the following claims.

We claim:

1. A method for providing a prognosis for a subject with prostate cancer, or selecting a subject with prostate cancer for treatment with a particular therapy, comprising:
detecting the level of expression of one or more genes selected from the group consisting of ACOT1, ARHGEF35, C17orf97, CAPN9, CCDC163P, DDX58, DDX60, ERAP2, IFIH1, KLF13, MICA, NOMO3, PAM, THNSL2, TMC4, and USP18 in a sample from the subject; and
comparing the level of expression of the one or more genes to a corresponding reference level of expression of the one or more genes, wherein an altered level of expression of the one or more genes relative to the reference level provides an indication selected from the group consisting of an indication of prostate cancer recurrence, an indication of survival of the subject, and an indication that the subject is a candidate for treatment with a particular therapy.

2. The method of claim 1, wherein an altered level of the genes in the sample as compared to the reference level is indicative of a decreased time to biochemical recurrence of prostate cancer in the subject.

3. A method for treating prostate cancer, comprising:
detecting the level of expression of one or more genes selected from the group consisting of ACOT1, ARHGEF35, C17orf97, CAPN9, CCDC163P, DDX58, DDX60, ERAP2, IFIH1, KLF13, MICA, NOMO3, PAM, THNSL2, TMC4, and USP18 in a sample from the subject; and
administering a prostate cancer treatment to a subject with an altered level of expression of the one or more genes.

4. A method for assaying gene expression in a sample from a subject diagnosed with prostate cancer, comprising:
detecting the level of expression of two or more genes selected from the group consisting of ACOT1, ARHGEF35, C17orf97, CAPN9, CCDC163P, DDX58, DDX60, ERAP2, IFIH1, KLF13, MICA, NOMO3, PAM, THNSL2, TMC4, and USP18 in the sample.

5. A method for stratifying a subject with prostate cancer comprising:
detecting the level of expression of one or more genes selected from the group consisting of ACOT1, ARHGEF35, C17orf97, CAPN9, CCDC163P, DDX58, DDX60, ERAP2, IFIH1, KLF13, MICA, NOMO3, PAM, THNSL2, TMC4, and USP18 in the sample; and
assigning a risk of prostate cancer recurrence to the subject based upon detection of an altered level of expression of the one or more genes.

6. The method of claim 5, wherein the risk of prostate cancer recurrence is a higher risk of recurrence as compared to a subject not exhibiting an altered level of the one or more genes.

7. The method of any one of claims 1 to 6, wherein the altered level of expression is an increased or decreased level of expression.

8. The method of claim 7, wherein the altered level of expression is an increased level of expression.

9. The method of claim 7, wherein the altered level of expression is a decreased level of expression.

10. The method of any of claims 1 to 9, wherein the one or more genes is two or more.

11. The method of any of claims 1 to 10 wherein the one or more genes is 5 or more.

12. The method of any of claims 1 to 11, wherein the one or more genes is all of the genes.

13. The method of any one of claims 1 to 12, wherein the sample is selected from the group consisting of prostate tissue, bone marrow, blood, serum, plasma, urine, prostatic fluid and semen.

14. The method of claim 13, wherein the prostate tissue is prostate cancer biopsy tissue.

15. The method of claim 13, wherein the sample comprises a prostate cancer cell.

16. The method of any of claims 1 to 15, wherein the subject has undergone surgery and/or radiotherapy.

17. The method of any of claims 1 to 16 wherein the detecting comprises the use of one or more nucleic acid reagents selected from the group consisting of a nucleic acid primers and nucleic acid probes and one or more antibodies.

18. The method of claim 17, wherein the primers, probes, and/or antibodies comprise a detectable label.

19. The method of any one of claims 1 to 18, wherein the subject has an already known high or intermediate risk of biochemical recurrence of cancer.

20. The method of any one of claims 1 to 19, wherein the methods further comprise stratifying the patients with an altered level of expression of one or more of the genes into an additional risk group.

21. The method of any one of claims 1 to 20, wherein the patient has previously undergone a radical prostatectomy.

22. The method of any one of claims 1 to 21, further comprising administering adjuvant treatment to the subject having an altered level expression of one or more of the genes.

23. The method of claim 22, wherein the adjuvant treatment is selected from the goup consisting of chemotherapy and androgen deprivation therapy and combinations thereof.

24. The method of claim 23, wherein the andogen deprivation therapy comprises administration of a luteinizing hormone-releasing hormone (LHRH) or gonadotropin-releasing hormone (GnRH) agonist or antagonist.

25. The method of claim 24, wherein the luteinizing hormone-releasing hormone (LHRH) or gonadotropin-releasing hormone (GnRH) agonist or antagonist is selected from the group consisting of Leuprolide, Goserelin, Triptorelin, Histrelin, and Degarelix.

26. The method of claim 23, wherein the androgen deprivation therapy comprises administration of an antiandrogen.

27. The method of claim 26, wherein the antiandrogen is selected from the group consisting of Bicalutamide, Nilutamide, and Flutamide.

28. Use of a reagent that specifically detects an altered level of expression of one or more genes selected from the group consisting of ACOT1, ARHGEF35, C17orf97, CAPN9, CCDC163P, DDX58, DDX60, ERAP2, IFIH1, KLF13, MICA, NOMO3, PAM, THNSL2, TMC4, and USP18 in a sample from a subject in the determination of the likelihood of survival of the subject or determining that the subject is a candidate for treatment with a particular therapy.

29. A kit for detecting altered levels of genes expression in a sample from a subject, comprising:
reagents that specifically detect two or more genes selected from the group consisting of ACOT1, ARHGEF35, C17orf97, CAPN9, CCDC163P, DDX58, DDX60, ERAP2, IFIH1, KLF13, MICA, NOMO3, PAM, THNSL2, TMC4, and USP18.

30. The kit of claim 29, wherein the reagents are selected from the group consisting of nucleic acid primers and nucleic acid probes.
